(19) United States
(12) Patent Application Publication
(10) Pub. No.: US 2020/0330555 A1
(43) Pub. Date: Oct. 22, 2020

(54) METHODS OF TREATING GASTROINTESTINAL MOTILITY-RELATED DISORDERS USING VARIANTS AND FUSIONS OF FGF19/FGF21 POLYPEPTIDES

(71) Applicant: NGM Biopharmaceuticals, Inc., South San Francisco, CA (US)

(72) Inventors: Stephen Joseph Rossi, South San Francisco, CA (US); Alexander Mark Deapoli, Santa Barbara, CA (US); Michael L. Camilleri, Rochester, MN (US)

(21) Appl. No.: 16/606,455

(22) PCT Filed: Apr. 20, 2018
(86) PCT No.: PCT/US2018/028512

§ 371 Date: Oct. 18, 2019

Related U.S. Application Data
(60) Provisional application No. 62/513,910, filed on Jun. 1, 2017.
(60) Provisional application No. 62/488,469, filed on Apr. 21, 2017.

(51) Int. Cl. A61K 38/18; A61P 1/10; C12Q 1/686

(52) U.S. Cl. CPC A61K 38/1825; C12Q 1/686; A61P 1/10

(57) ABSTRACT

Provided herein are methods of treating or preventing gastrointestinal motility-related disorders, treating or preventing constipation or stimulating bowel function, comprising using variants and fusions of fibroblast growth factor 19 (FGF19), variants and fusions of fibroblast growth factor 21 (FGF21), fusions of FGF19 and/or FGF21, and variants or fusions of FGF19 and/or FGF21 proteins and peptide sequences (and peptidomimetics).

### CROSS-REFERENCE TO RELATED APPLICATIONS

This application is a U.S. National Stage Application under 35 U.S.C. § 371 of International Patent Application No. PCT/US2018/028512 filed Apr. 20, 2018, which claims the benefit of priority to U.S. Ser. No. 62/488,469 filed Apr. 21, 2017 and to U.S. Ser. No. 62/513,910 filed Jun. 1, 2017, the content of each of which is incorporated herein by reference in its entirety.

### FIELD

The invention relates, in part, to the treatment or prevention of gastrointestinal motility-related disorders with variants of fibroblast growth factor 19 (FGF19) proteins and peptide sequences (and peptidomimetics) and fusions of FGF19 and/or fibroblast growth factor 21 (FGF21) proteins and peptide sequences (and peptidomimetics), and variants of fusions of FGF19 and/or FGF21 proteins and peptide sequences (and peptidomimetics).

### INTRODUCTION

Gastrointestinal motility-related disorders include a wide variety of motility disorders affecting the gastrointestinal (“GI”) tract which include, for example, constipation, irritable bowel syndrome and gastroparesis. A large percentage of the population suffers from gastrointestinal motility-related disorders. Constipation is the most common chronic gastrointestinal motility-related disorder in adults. In the general population, rates of constipation are estimated to be between 2-30%. Among elderly people living in a care home, the rate of constipation can be between 50-75%. Constipation results in a large economic burden in the United States and other countries. For example, an average of about 2,500,000 visits to physicians for constipation occurs annually in the United States, with more than $250 million spending on constipation medications.

Although there can be a number of contributing factors, the cause of constipation and other gastrointestinal motility-related disorders is unknown in a large number of patients. Current therapeutic modalities for treating or preventing gastrointestinal motility-related disorders usually focus on the underlying cause if known, and many patients are inadequately treated and can be benefit from new and more effective treatment regimens. Therefore, methods to treat or prevent gastrointestinal motility-related disorders, especially in patients with unknown causes, represent unmet needs. The methods and compositions of the present invention meet these needs and provide other related advantages.

### SUMMARY

The invention is based, in part, on the use of variants of FGF19 peptide sequences, fusions of FGF19 and/or FGF21 peptide sequences and variants of fusions (chimeras) of FGF19 and/or FGF21 peptide sequences having one or more activities associated with the treatment and/or prevention of gastrointestinal motility-related disorders, such as constipation. Such variants and fusions (chimeras) of FGF19 and/or FGF21 peptide sequences include sequences that do not substantially increase or induce hepatocellular carcinoma (HCC) formation or HCC tumorigenesis and/or do not induce a substantial elevation or increase in lipid profile. Examples of such variants and fusions (chimeras) of FGF19 and/or FGF21 peptide sequences further include those sequences disclosed in PCT Pub. No. WO 2013/006486 and US Pub. No. 2013/0023474, published Jan. 20, 2013 and Jan. 24, 2013, respectively; as well as PCT Publ. No. WO 2014/085365, published Jun. 5, 2014.

Provided herein are compositions comprising certain peptide sequences, including subsequences, variants and modified forms of the exemplified peptide sequences (including the FGF19 and FGF21 variants and subsequences listed in the Sequence Listing or Table 1, and the FGF19/FGF21 fusions and chimeras listed in the Sequence Listing or Table 1), and one or more pharmaceutically acceptable carriers or excipients. Combinations, such as one or more peptide sequences in a pharmaceutically acceptable carrier or excipient, with one or more therapeutic agents or treatment modalities useful in the treatment and/or prevention of gastrointestinal motility-related disorders are also provided. Such combinations of peptide sequence(s) provided herein with one or more additional agents or modalities are useful in accordance with the methods and uses provided herein.

Uses and methods of treatment or prevention that include administration or delivery of a chimeric peptide or peptide sequence in combination with an agent that stimulates bowel function are also provided herein. In particular embodiments, a use or method of treatment or prevention in a subject includes administering a chimeric peptide or peptide sequence provided herein to a subject having, or at risk of having, a gastrointestinal motility-related disorder treatable by a peptide sequence provided herein, in an amount effective for treating the disorder. In particular embodiments, a use or method of treatment or prevention in a subject includes administering a chimeric peptide or peptide sequence provided herein to a subject having, or at risk of having, a disorder of constipation or inadequate bowel function treatable by a peptide sequence provided herein, in an amount effective for treating the disorder. In some embodiments of the various methods provided herein, the methods are methods of treatment. In other embodiments of the various methods provided herein, the methods are methods of prevention.

In one embodiment, a method or use of treating or preventing a gastrointestinal motility-related disorder in a subject includes: administering a therapeutically effective amount of a chimeric peptide sequence, comprising: i) an N-terminal region comprising at least seven amino acid residues, the N-terminal region having a first amino acid position and a last amino acid position, wherein the N-terminal region comprises DSSPL (SEQ ID NO:121) or DASPH (SEQ ID NO:122), and ii) a C-terminal region comprising a portion of SEQ ID NO:99 (FGF19), the C-terminal region having a first amino acid position and a last amino acid position, wherein the C-terminal region comprises amino acid residues 16-29 of SEQ ID NO:99 (FGF19), WGDPIRLRHLYTSG (SEQ ID NO:169), wherein the W residue corresponds to the first amino acid position of the C-terminal region, thereby treating or preventing the gastrointestinal motility-related disorder in the subject.

In another embodiment, a method or use of treating or preventing a gastrointestinal motility-related disorder in a subject includes: administering a therapeutically effective amount of a chimeric peptide sequence, comprising: i) an N-terminal region comprising a portion of SEQ ID NO:100 (FGF21), the N-terminal region having a first amino acid position and a last amino acid position, wherein the N-terminal region comprises amino acid residues GQV, and wherein the V residue corresponds to the last amino acid position of the N-terminal region, and ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region; thereby treating or preventing the gastrointestinal motility-related disorder in the subject.

In a further embodiment, a method or use of treating or preventing a gastrointestinal motility-related disorder in a subject includes: administering a therapeutically effective amount of a chimeric peptide sequence comprising: i) an N-terminal region comprising a portion of SEQ ID NO:100 (FGF21), the N-terminal region having a first amino acid position and a last amino acid position, wherein the N-terminal region comprises at least 5 contiguous amino acids of SEQ ID NO:100 (FGF21) including the amino acid residues GQV, and wherein the V residue corresponds to the last amino acid position of the N-terminal region, and ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region; thereby treating or preventing the gastrointestinal motility-related disorder in the subject.

In an additional embodiment, a method or use of treating or preventing a gastrointestinal motility-related disorder in a subject includes: administering a therapeutically effective amount of a peptide sequence, comprising or consisting of any of: i) a FGF19 sequence variant having one or more amino acid substitutions, insertions or deletions compared to a reference or wild type FGF19, ii) a FGF21 sequence variant having one or more amino acid substitutions, insertions or deletions compared to a reference or wild type FGF21, iii) a portion of a FGF19 sequence fused to a portion of a FGF21 sequence, or iv) a portion of a FGF19 sequence fused to a portion of a FGF21 sequence, wherein the FGF19 and/or FGF21 sequence portion(s) have one or more amino acid substitutions, insertions or deletions compared to a reference or wild type FGF19 and/or FGF21; thereby treating or preventing the gastrointestinal motility-related disorder in the subject.

In one embodiment, a method or use of treating or preventing constipation in a subject includes: administering a therapeutically effective amount of a chimeric peptide sequence, comprising: i) an N-terminal region comprising at least seven amino acid residues, the N-terminal region having a first amino acid position and a last amino acid position, wherein the N-terminal region comprises DSSPL (SEQ ID NO:121) or DASPH (SEQ ID NO:122), and ii) a C-terminal region comprising a portion of SEQ ID NO:99 (FGF19), the C-terminal region having a first amino acid position and a last amino acid position, wherein the C-terminal region comprises amino acid residues 16-29 of SEQ ID NO:99 (FGF19), WGDPIRLRHLYTSG (SEQ ID NO:169), wherein the W residue corresponds to the first amino acid position of the C-terminal region, thereby treating or preventing constipation in the subject.

In another embodiment, a method or use of treating or preventing constipation in a subject includes: administering a therapeutically effective amount of a chimeric peptide sequence, comprising: i) an N-terminal region comprising a portion of SEQ ID NO:100 (FGF21), the N-terminal region having a first amino acid position and a last amino acid position, wherein the N-terminal region comprises amino acid residues GQV, and wherein the V residue corresponds to the last amino acid position of the N-terminal region, and ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region; thereby treating or preventing constipation in the subject.

In a further embodiment, a method or use of treating or preventing constipation in a subject includes: administering a therapeutically effective amount of a chimeric peptide sequence comprising: i) an N-terminal region comprising a portion of SEQ ID NO:100 (FGF21), the N-terminal region having a first amino acid position and a last amino acid position, wherein the N-terminal region comprises at least 5 contiguous amino acids of SEQ ID NO:100 (FGF21) including the amino acid residues GQV, and wherein the V residue corresponds to the last amino acid position of the N-terminal region, and ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region; thereby treating or preventing constipation in the subject.

In an additional embodiment, a method or use of treating or preventing constipation in a subject includes: administering a therapeutically effective amount of a peptide sequence, comprising or consisting of any of: i) a FGF19 sequence variant having one or more amino acid substitutions, insertions or deletions compared to a reference or wild type FGF19, ii) a FGF21 sequence variant having one or more amino acid substitutions, insertions or deletions compared to a reference or wild type FGF21, iii) a portion of a FGF19 sequence fused to a portion of a FGF21 sequence, or iv) a portion of a FGF19 sequence fused to a portion of a FGF21 sequence, wherein the FGF19 and/or FGF21 sequence portion(s) have one or more amino acid substitutions, insertions or deletions compared to a reference or wild type FGF19 and/or FGF21; thereby treating or preventing constipation in the subject.

In various particular embodiments, a chimeric peptide sequence has an N-terminal region with at least 6 contiguous amino acids of SEQ ID NO:100 (FGF21) including the amino acid residues GQ; or has an N-terminal region with at least 7 contiguous amino acids of SEQ ID NO:100 (FGF21) including the amino acid residues GQV.

In one embodiment, a method or use of stimulating bowel function in a subject includes: administering a therapeutically effective amount of a chimeric peptide sequence, comprising: i) an N-terminal region comprising at least seven amino acid residues, the N-terminal region having a first amino acid position and a last amino acid position, wherein the N-terminal region comprises DSSPL (SEQ ID NO:121) or DASPH (SEQ ID NO:122), and ii) a C-terminal region comprising a portion of SEQ ID NO:99 (FGF19), the C-terminal region having a first amino acid position and a last amino acid position, wherein the C-terminal region comprises amino acid residues 16-29 of SEQ ID NO:99 (FGF19), WGDPIRLRHLYTSG (SEQ ID NO:169), wherein the W residue corresponds to the first amino acid position of the C-terminal region.

In another embodiment, a method or use of stimulating bowel function in a subject includes: administering a therapeutically effective amount of a chimeric peptide sequence, comprising: i) an N-terminal region comprising a portion of SEQ ID NO:100 (FGF21), the N-terminal region having a first amino acid position and a last amino acid position, wherein the N-terminal region comprises amino acid residues GQV, and wherein the V residue corresponds to the last amino acid position of the N-terminal region, and ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region.

In a further embodiment, a method or use of stimulating bowel function in a subject includes: administering a therapeutically effective amount of a chimeric peptide sequence comprising: i) an N-terminal region comprising a portion of SEQ ID NO:100 (FGF21), the N-terminal region having a first amino acid position and a last amino acid position, wherein the N-terminal region comprises at least 5 contiguous amino acids of SEQ ID NO:100 (FGF21) including the amino acid residues GQV, and wherein the V residue corresponds to the last amino acid position of the N-terminal region, and ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region.

In an additional embodiment, a method or use of stimulating bowel function in a subject includes: administering a therapeutically effective amount of a peptide sequence, comprising or consisting of any of: i) a FGF19 sequence variant having one or more amino acid substitutions, insertions or deletions compared to a reference or wild type FGF19, ii) a FGF21 sequence variant having one or more amino acid substitutions, insertions or deletions compared to a reference or wild type FGF21, iii) a portion of a FGF19 sequence fused to a portion of a FGF21 sequence, or iv) a portion of a FGF19 sequence fused to a portion of a FGF21 sequence, wherein the FGF19 and/or FGF21 sequence portion(s) have one or more amino acid substitutions, insertions or deletions compared to a reference or wild type FGF19 and/or FGF21.

In various particular embodiments, a chimeric peptide sequence has an N-terminal region with at least 6 contiguous amino acids of SEQ ID NO:100 (FGF21) including the amino acid residues GQ; or has an N-terminal region with at least 7 contiguous amino acids of SEQ ID NO:100 (FGF21) including the amino acid residues GQV.

In various additional embodiments, a peptide sequence has amino-terminal amino acids 1-16 of SEQ ID NO:100 (FGF21) fused to carboxy-terminal amino acids 21-194 of SEQ ID NO:99 (FGF19), or the peptide sequence has amino-terminal amino acids 1-147 of SEQ ID NO:99 (FGF19) fused to carboxy-terminal amino acids 147-181 of SEQ ID NO:100 (FGF21) (M41), or the peptide sequence has amino-terminal amino acids 1-20 of SEQ ID NO:99 (FGF19) fused to carboxy-terminal amino acids 17-181 of SEQ ID NO:100 (FGF21) (M44), or the peptide sequence has amino-terminal amino acids 1-146 of SEQ ID NO:100 (FGF21) fused to carboxy-terminal amino acids 148-194 of SEQ ID NO:99 (FGF19) (M45), or the peptide sequence has amino-terminal amino acids 1-20 of SEQ ID NO:99 (FGF19) fused to internal amino acids 17-146 of SEQ ID NO:100 (FGF21) or fused to carboxy-terminal amino acids 148-194 of SEQ ID NO:99 (FGF19) (M46).

In various further embodiments, a peptide sequence has at least one amino acid substitution to amino acid residues 125-129 of SEQ ID NO:99 (FGF19), EIRPD; at least one amino acid substitution to amino acid residues 126-128 of SEQ ID NO:99 (FGF19), IRP; or at least one amino acid substitution to amino acid residues 127-128 of SEQ ID NO:99 (FGF19), RP, or at least one amino acid substitution to amino acid residues 1-124 of SEQ ID NO:99 (FGF19) and/or to amino acid residues 130-194 of SEQ ID NO:99 (FGF19). More specifically, for example, a peptide sequence with a substitution to one of amino acid residues 127-128 of SEQ ID NO:99 (FGF19), RP, wherein at least one amino acid substitution is R127L or P128E. Said substitutions within a corresponding FGF19 sequence (e.g., EIRPD, IRP or RP) of a peptide variant provided herein is also contemplated. In certain embodiments, the peptide comprises both a R127L and P128E substitution to amino acid residues 127-128 of SEQ ID NO:99 (FGF19), RP, or the corresponding FGF19 sequence thereof in a variant peptide provided herein. In certain embodiments, the amino acid sequence of the peptide comprises at least one amino acid substitution in the Loop-8 region of FGF19, or the corresponding FGF19 sequence thereof in a variant peptide provided herein. In certain embodiments, the amino acid sequence of the peptide comprises one amino acid substitution to the EIRPD (amino acids 2-6 of SEQ ID NO:190) amino acid sequence in the Loop-8 region of FGF19. In some embodiments, the amino acid sequence of the peptide comprises two amino acid substitutions to the EIRPD (amino acids 2-6 of SEQ ID NO:190) amino acid sequence in the Loop-8 region of FGF19. In other embodiments, the amino acid sequence of the peptide comprises three amino acid substitutions to the EIRPD (amino acids 2-6 of SEQ ID NO:190) amino acid sequence in the Loop-8 region of FGF19. In certain embodiments, the amino acid sequence of the peptide comprises four amino acid substitutions to the EIRPD (amino acids 2-6 of SEQ ID NO:190) amino acid sequence in the Loop-8 region of FGF19. In some embodiments, the amino acid sequence of the peptide comprises five amino acid substitutions to the EIRPD (amino acids 2-6 of SEQ ID NO:190) amino acid sequence in the Loop-8 region of FGF19. In certain embodiments, the amino acid sequence of the peptide comprises one amino acid substitution to the IRP (amino acids 3-5 of SEQ ID NO:190) amino acid sequence in the Loop-8 region of FGF19. In some embodiments, the amino acid sequence of the peptide comprises two amino acid substitutions to the IRP (amino acids 3-5 of SEQ ID NO:190) amino acid sequence in the Loop-8 region of FGF19. In other embodiments, the amino acid sequence of the peptide comprises three amino acid substitutions to the IRP (amino acids 3-5 of SEQ ID NO:190) amino acid sequence in the Loop-8 region of FGF19. In certain embodiments, the amino acid sequence of the peptide comprises one amino acid substitution to the RP (amino acids 4-5 of SEQ ID NO:190) amino acid sequence in the Loop-8 region of FGF19. In some embodiments, the amino acid sequence of the peptide comprises two amino acid substitutions to the RP (amino acids 4-5 of SEQ ID NO:190) amino acid sequence in the Loop-8 region of FGF19. In certain embodiments, the amino acid substitution to the RP (amino acids 4-5 of SEQ ID NO:190) amino acid sequence in the Loop-8 region of FGF19 is an Arg (R) to Leu (L) substitution. In other embodiments, the substitution to the RP (amino acids 4-5 of SEQ ID NO:190) amino acid sequence in the Loop-8 region of FGF19 is a Pro (P) to Glu (E) substitution. In some embodiments, the substitutions to the RP (amino acids 4-5 of SEQ ID NO:190) amino acid sequence in the Loop-8 region of FGF19 is an Arg (R) to Leu (L) substitution and a Pro (P) to Glu (E) substitution. In specific embodiments, the foregoing substitution(s) in the Loop-8 region of FGF19 is in the corresponding FGF19 sequence thereof in a variant peptide provided herein. That is, said substitutions within a corresponding FGF19 sequence (e.g., EIRPD, IRP or RP) of a peptide variant provided herein is also contemplated.

Methods and uses provided herein can be practiced using a peptide or chimeric sequence, as set forth herein. For example, a sequence that comprises or consists of any peptide sequence set forth herein as M1 to M98, M101 to M160, or M200 to M207, or SEQ ID NOS: 1 to 98, 138 to 168, or 192 to 204. In other embodiments, the peptide sequence comprises or consists of any sequence set forth in Table 1. In yet other embodiments, the peptide sequence comprises or consists of any sequence set forth in the Sequence Listing herein.

Methods and uses provided herein can be practiced using a peptide or chimeric sequence of any suitable length. In particular embodiments, the N-terminal or C-terminal region of the peptide or chimeric sequence is from about 20 to about 200 amino acid residues in length. In other particular aspects, a peptide or chimeric sequence has 1, 2, 3, 4, 5, 6, 7, 8, 9, 10, 11, 12, 13, 14, 15, 16, 17, 18, 19, 20 or more amino acid deletions from the amino terminus, the carboxy-terminus or internally. In further particular embodiments, a peptide or chimeric sequence has an N-terminal region, or a C-terminal region that comprises or consists of an amino acid sequence of about 5 to 10, 10 to 20, 20 to 30, 30 to 40, 40 to 50, 60 to 70, 70 to 80, 80 to 90, 90 to 100 or more amino acids. In additional more particular embodiments, a peptide or chimeric sequence has a FGF19 sequence portion, or a FGF21 sequence portion that comprises or consists of an amino acid sequence of about 5 to 10, 10 to 20, 20 to 30, 30 to 40, 40 to 50, 50 to 60, 60 to 70, 70 to 80, 80 to 90, 90 to 100 or more amino acids of FGF19 or FGF21.

In yet additional embodiments, a peptide sequence or a chimeric peptide sequence has a WGDPI (SEQ ID NO:170) sequence motif corresponding to the WGDPI (SEQ ID NO:170) sequence of amino acids 16-20 of SEQ ID NO:99 (FGF19); has a substituted, mutated or absent WGDPI (SEQ ID NO:170) sequence motif corresponding to FGF19 WGDPI (SEQ ID NO:170) sequence of amino acids 16-20 of FGF19; has a WGDPI (SEQ ID NO:170) sequence with one or more amino acids substituted, mutated or absent. In various other further aspects, the peptide sequence is distinct from a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the FGF19 WGDPI (SEQ ID NO:170) sequence at amino acids 16-20.

In yet further embodiments, a peptide sequence or a chimeric peptide sequence has N-terminal region comprises amino acid residues VHYG (SEQ ID NO:101), wherein the N-terminal region comprises amino acid residues DASPHVHYG (SEQ ID NO:102), or the N-terminal region comprises amino acid residues DSSPLVHYG (SEQ ID NO:103). More particularly, in one aspect the G corresponds to the last position of the N-terminal region.

In various additional aspects, the N-terminal region comprises amino acid residues DSSPLLQ (SEQ ID NO:104), where the Q residue is the last amino acid position of the N-terminal region, or comprises amino acid residues DSSPLLQFGGQV (SEQ ID NO:105), where the V residue corresponds to the last position of the N-terminal region.

In certain embodiments, an N-terminal region comprises or consists of (or further comprises or consists of): RHPIP (SEQ ID NO:106), where R is the first amino acid position of the N-terminal region; or HPIP (SEQ ID NO:107), where H is the first amino acid position of the N-terminal region; or RPLAF (SEQ ID NO:108), where R is the first amino acid position of the N-terminal region; or PLAF (SEQ ID NO:109), where P is the first amino acid position of the N-terminal region; or R, where R is the first amino acid position of the N-terminal region.

In various other aspects, a peptide or chimeric sequence has: amino acid residues HPIP (SEQ ID NO:107), which are the first 4 amino acid residues of the N-terminal region. In still further aspects, a peptide or chimeric sequence has: an R residue at the first position of the N-terminal region, or the first position of the N-terminal region is an M residue, or the first and second positions of the N-terminal region is an MR sequence, or the first and second positions of the N-terminal region is an RM sequence, or the first and second positions of the N-terminal region is an RD sequence, or the first and second positions of the N-terminal region is an DS sequence, or the first and second positions of the N-terminal region is an MD sequence, or the first and second positions of the N-terminal region is an MS sequence, or the first through third positions of the N-terminal region is an MDS sequence, or the first through third positions of the N-terminal region is an RDS sequence, or the first through third positions of the N-terminal region is an MSD sequence, or the first through third positions of the N-terminal region is an MSS sequence, or the first through third positions of the N-terminal region is an DSS sequence, or the first through fourth positions of the N-terminal region is an RDSS (SEQ ID NO:115), sequence, or the first through fourth positions of the N-terminal region is an MDSS (SEQ ID NO:116), sequence, or the first through fifth positions of the N-terminal region is an MRDSS (SEQ ID NO:117), sequence, or the first through fifth positions of the N-terminal region is an MSSPL (SEQ ID NO:113) sequence, or the first through sixth positions of the N-terminal region is an MDSSPL (SEQ ID NO:110) sequence, or the first through seventh positions of the N-terminal region is an MSDSSPL (SEQ ID NO:111) sequence.

In various other particular aspects, a peptide or chimeric sequence has at the N-terminal region first amino acid position an “M” residue, an “R” residue, an “S” residue, a “H” residue, a “P” residue, a “L” residue or an “D” residue. In various alternative particular aspects, a peptide or chimeric sequence peptide sequence does not have a “M” residue or an “R” residue at the first amino acid position of the N-terminal region.

In further various other aspects, a peptide or chimeric sequence has an N-terminal region with any one of the following sequences: MDSSPL (SEQ ID NO:110), MSDSSPL (SEQ ID NO:111), SDSSPL (SEQ ID NO:112), MSSPL (SEQ ID NO:113) or SSPL (SEQ ID NO:114).

In still additional aspects, a peptide sequence or chimeric peptide sequence has a residue at the last position of the C-terminal region that corresponds to about residue 194 of SEQ ID NO:99 (FGF19). In still other embodiments, a peptide sequence or a chimeric peptide sequence an addition of amino acid residues 30-194 of SEQ ID NO:99 (FGF19) at the C-terminus, resulting in a chimeric polypeptide having a residue at the last position of the C-terminal region that corresponds to about residue 194 of SEQ ID NO:99 (FGF19). In further other embodiments, a chimeric peptide sequence or peptide sequence comprises all or a portion of a FGF19 sequence (e.g., SEQ ID NO:99), positioned at the C-terminus of the peptide, or where the amino terminal “R” residue is deleted from the peptide.

In more particular embodiments, a chimeric peptide sequence or peptide sequence comprises or consists of any of M1-M98 variant peptide sequences, or a subsequence or fragment of any of the M1-M98 variant peptide sequences. Methods and uses provided herein can also be practiced using a peptide or chimeric sequence, as set forth herein. For example, a sequence that comprises or consists of any peptide sequence set forth herein as M1 to M98, M101 to M160, or M200 to M207 or SEQ ID NOs: 1 to 98, 138 to 168, or 192 to 204, or a peptide sequence that comprises or consists of any sequence set forth in Table 1, or a peptide sequence that comprises or consists of any sequence set forth in the Sequence Listing herein.

In various more particular aspects, a peptide sequence comprises or consists of any one of the following sequences:

(SEQ ID NO: 3)
RPLAFSDAGPHVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEILEDGYNVYRSEKHRLPVSLSSAKQRQLYKNRGFLPLSHFLPMLPMV
PEEPEDLRGHLESDMFSSPLETDSMDPFGLVTGLEAVRSPSFEK (M3);
(SEQ ID NO: 194)
RPLAFSDAGPHVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEIREDGYNVYRSEKHRLPVSLSSAKQRQLYKNRGFLPLSHFLPMLPMV
PEEPEDLRGHLESDNIFSSPLETDSMDPFGLVTGLEAVRSPSFEK
(M140);
(SEQ ID NO: 196)
RPLAFSDAGPHVHYGWGDPIRQRHLYTSGPHGLSSCFLRIRADGVVDCAR
GQSAHSLLEIKAVALRTVAIKGVHSVRYLCMGADGKMQGLLQYSEEDCAF
EEEILEDGYNVYRSEKHRLPVSLSSAKQRQLYKNRGFLPLSHFLPMLPMV
PEEPEDLRGHLESDMFSSPLETDSMDPFGLVTGLEAVRSPSFEK
(M160);
(SEQ ID NO: 69)
RDSSPLVHYGWGDPIRLRHLYTSGPHGLSSCFLRIRADGVVDCARGQSAH
SLLEIKAVALRTVAIKGVHSVRYLCMGADGKMQGLLQYSEEDCAFEEEIR
PDGYNVYRSEKHRLPVSLSSAKQRQLYKNRGFLPLSHFLPMLPMVPEEPE
DLRGHLESDMFSSPLETDSMDPFGLVTGLEAVRSPSFEK (M69);
(SEQ ID NO: 52)
RDSSPLLQWGDPIRLRHLYTSGPHGLSSCFLRIRADGVVDCARGQSAHSL
LEIKAVALRTVAIKGVHSVRYLCMGADGKMQGLLQYSEEDCAFEEEIRPD
GYNVYRSEKHRLPVSLSSAKQRQLYKNRGFLPLSHFLPMLPMVPEEPEDL
RGHLESDMFSSPLETDSMDPFGLVTGLEAVRSPSFEK (M52);
(SEQ ID NO: 5)
RHPIPDSSPLLQFGGQVRLRHLYTSGPHGLSSCFLRIRADGVVDCARGQS
AHSLLEIKAVALRTVAIKGVHSVRYLCMGADGKMQGLLQYSEEDCAFEEE
IRPDGYNVYRSEKHRLPVSLSSAKQRQLYKNRGFLPLSHFLPMLPMVPEE
PEDLRGHLESDMFSSPLETDSMDPFGLVTGLEAVRSPSFEK (M5);
(SEQ ID NO: 160)
HPIPDSSPLLQFGGQVRLRHLYTSGPHGLSSCFLRIRADGVVDCARGQSA
HSLLEIKAVALRTVAIKGVHSVRYLCMGADGKMQGLLQYSEEDCAFEEEI
RPDGYNVYRSEKHRLPVSLSSAKQRQLYKNRGFLPLSHFLPMLPMVPEEP
EDLRGHLESDMFSSPLETDSMDPFGLVTGLEAVRSPSFEK (M5-R);
(SEQ ID NO: 71)
HPIPDSSPLLQFGGQVRQRYLYTDDAQQTEAHLEIREDGTVGGAADQSPE
SLLQLKALKPGVIQILGVKTSRFLCQRPDGALYGSLHFDPEACSFRELLL
EDGYNVYQSEAHSLPLHLPGNKSPHRDPAPRGPARFLPLPGLPPALPEPP
GILAPQPPDVGSSDPLSMVGPSQGRSPSYAS (M71);
(SEQ ID NO: 72)
HPIPDSSPLLQFGGQVRQRYLYTDDAQQTEAHLEIREDGTVGGAADQSPE
SLLQLKALKPGVIQILGVKTSRFLCQRPDGALYGSLHFDPEACSFRELLL
EDGYNVYQSEAHGLPLHLPGNKSPHRDPAPRGPARFLPLPGLPPAPPEPP
GILAPQPPDVGSSDPLSMVGPSQGRSPSYAS (M72);
(SEQ ID NO: 73)
HPIPDSSPLLQFGGQVRQRYLYTDDAQQTEAHLEIREDGTVGGAADQSPE
SLLQLKALKPGVIQILGVKTSRFLCQRPDGALYGSLHFDPEACSFRELLL
EDGYNVYQSEAHGLPLHLPGNKSPHRDPAPRGPARFLPLPGLPPALPEPP
GILAPQPPDVGSSDPLSMVVQDELQGVGGEGCHMHPENCKTLLTDIDRTH
TEKPVWDGITGE (M73);
(SEQ ID NO: 1 or 139)
RPLAFSDASPHVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEIRPDGYNVYRSEKHRLPVSLSSAKQRQLYKNRGFLPLSHFLPMLPMV
PEEPEDLRGHLESDMFSSPLETDSMDPFGLVTGLEAVRSPSFEK (M1);
(SEQ ID NO: 2 or 140)
RPLAFSDSSPLVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEIRPDGYNVYRSEKHRLPVSLSSAKQRQLYKNRGFLPLSHFLPMLPMV
PEEPEDLRGHLESDMFSSPLETDSMDPFGLVTGLEAVRSPSFEK (M2);
(SEQ ID NO: 48 or 6 or 148)
RDSSPLLQFGGQVRLRHLYTSGPHGLSSCFLRIRADGVVDCARGQSAHSL
LEIKAVALRTVAIKGVHSVRYLCMGADGKMQGLLQYSEEDCAFEEEIRPD
GYNVYRSEKHRLPVSLSSAKQRQLYKNRGFLPLSHFLPMLPMVPEEPEDL
RGHLESDMFSSPLETDSMDPFGLVTGLEAVRSPSFEK (M48);
(SEQ ID NO: 49 or 7 or 149)
RPLAFSDSSPLLQFGGQVRLRHLYTSGPHGLSSCFLRIRADGVVDCARGQ
SAHSLLEIKAVALRTVAIKGVHSVRYLCMGADGKMQGLLQYSEEDCAFEE
EIRPDGYNVYRSEKHRLPVSLSSAKQRQLYKNRGFLPLSHFLPMLPMVPE
EPEDLRGHLESDMFSSPLETDSMDPFGLVTGLEAVRSPSFEK (M49);
(SEQ ID NO: 50)
RHPIPDSSPLLQFGDQVRLRHLYTSGPHGLSSCFLRIRADGVVDCARGQS
AHSLLEIKAVALRTVAIKGVHSVRYLCMGADGKMQGLLQYSEEDCAFEEE
ILEDGYNVYRSEKHRLPVSLSSAKQRQLYKNRGFLPLSHFLPMLPMVPEE
PEDLRGHLESDMFSSPLETDSMDPFGLVTGLEAVRSPSFEK (M50);
(SEQ ID NO: 51 or 36 or 155)
RHPIPDSSPLLQFGGNVRLRHLYTSGPHGLSSCFLRIRADGVVDCARGQS
AHSLLEIKAVALRTVAIKGVHSVRYLCMGADGKMQGLLQYSEEDCAFEEE
IRPDGYNVYRSEKHRLPVSLSSAKQRQLYKNRGFLPLSHFLPMLPMVPEE
PEDLRGHLESDMFSSPLETDSMDPFGLVTGLEAVRSPSFEK (M51);
(SEQ ID NO: 192)
MDSSPLLQWGDPIRLRHLYTSGPHGLSSCFLRIRADGVVDCARGQSAHSL
LEIKAVALRTVAIKGVHSVRYLCMGADGKMQGLLQYSEEDCAFEEEIRPD
GYNVYRSEKHRLPVSLSSAKQRQLYKNRGFLPLSHFLPMLPMVPEEPEDL
RGHLESDMFSSPLETDSMDPFGLVTGLEAVRSPSFEK (M53);
(SEQ ID NO: 70)
MRDSSPLVHYGWGDPIRLRHLYTSGPHGLSSCFLRIRADGVVDCARGQSA
HSLLEIKAVALRTVAIKGVHSVRYLCMGADGKMQGLLQYSEEDCAFEEEI
RPDGYNVYRSEKHRLPVSLSSAKQRQLYKNRGFLPLSHFLPMLPMVPEEP
EDLRGHLESDMFSSPLETDSMDPFGLVTGLEAVRSPSFEK (M70);
(SEQ ID NO: 193)
RPLAFSDAGPHVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEILPDGYNVYRSEKHRLPVSLSSAKQRQLYKNRGFLPLSHFLPMLPMV
PEEPEDLRGHLESDMFSSPLETDSMDPFGLVTGLEAVRSPSFEK
(M139);
or
(SEQ ID NO: 195)
RPLAFSDAGPHVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEILCDGYNVYRSEKHRLPVSLSSAKQRQLYKNRGFLPLSHFLPMLPMV
PEEPEDLRGHLESDMFSSPLETDSMDPFGLVTGLEAVRSPSFEK
(M141);
RPLAFSDAGPHVHYGWGDPIRQRHLYTSGPHGLSSCFLRIRADGVVDCAR
GQSAHSLLEIKAVALRTVAIKGVHSVRYLCMGADGKMQGLLQYSEEDCAF
EEEILEDGYNVYRSEKHRLPVSLSSAKQRQLYKNRGFLPLSHFLPMLPMV
PEEPEDLRGHLESDMFSSPLETDSMDPFGLVTGLEAVRSPSFEK
(M160);

or a subsequence or fragment thereof of any of the foregoing peptide sequences. In certain embodiments of any of the foregoing peptide sequences, the R terminal residue (R residue at the N-terminus) is deleted.

In other embodiments, the peptide comprises or consists of: RDSSPLVHYGWGDPIRLRHLYTSGPHGLSSCFLRIRADGVVDCARGQSAHSLLEIKAVA LRTVAIKGVHSVRYLCMGADGKMQGLLQYSEEDCAFEEEILEDGYNVYRSEKHRLPVS LSSAKQRQLYKNRGFLPLSHFLPMLPMVPEEPEDLRGHLESDMFSSPLETDSMDPFGLVT GLEAVRSPSFEK (M200) (SEQ ID NO:197); or a subsequence or fragment thereof. In one embodiment, the N-terminal R residue is deleted.

In some embodiments, the peptide comprises or consists of: RPLAFSDSSPLVHYGWGDPIRLRHLYTSGPHGLSSCFLRIRADGVVDCARGQSAHSLLEI KAVALRTVAIKGVHSVRYLCMGADGKMQGLLQYSEEDCAFEEEILEDGYNVYRSEKHR LPVSLSSAKQRQLYKNRGFLPLSHFLPMLPMVPEEPEDLRGHLESDMFSSPLETDSMDPF GLVTGLEAVRSPSFEK (M201) (SEQ ID NO:198); or a subsequence or fragment thereof. In one embodiment, the N-terminal R residue is deleted.

In certain embodiments, the peptide comprises or consists of: RPLAF SDASPHVHYGWGDPIRLRHLYT SGPHGL S SCFLRIRADGVVDCARGQ SAHSLLEI KAVALRTVAIKGVHSVRYLCMGADGKMQGLLQYSEEDCAFEEEILEDGYNVYRSEKHR LPVSLSSAKQRQLYKNRGFLPLSHFLPMLPMVPEEPEDLRGHLESDMFSSPLETDSMDPF GLVTGLEAVRSPSFEK (M202) (SEQ ID NO:199); or a subsequence or fragment thereof. In one embodiment, the N-terminal R residue is deleted.

In other embodiments, the peptide comprises or consists of: RDSSPLLQWGDPIRLRHLYTSGPHGLSSCFLRIRADGVVDCARGQSAHSLLEIKAVALRT VAIKGVHSVRYLCMGADGKMQGLLQYSEEDCAFEEEILEDGYNVYRSEKHRLPVSLSS AKQRQLYKNRGFLPLSHFLPMLPMVPEEPEDLRGHLESDMFSSPLETDSMDPFGLVTGL EAVRSPSFEK (M203) (SEQ ID NO:200); or a subsequence or fragment thereof. In one embodiment, the N-terminal R residue is deleted.

In some embodiments, the peptide comprises or consists of: RHPIPDSSPLLQFGDQVRLRHLYTSGPHGLSSCFLRIRADGVVDCARGQSAHSLLEIKAV ALRTVAIKGVHSVRYLCMGADGKMQGLLQYSEEDCAFEEEILEDGYNVYRSEKHRLPV SLSSAKQRQLYKNRGFLPLSHFLPMLPMVPEEPEDLRGHLESDNIFSSPLETDSMDPFGLV TGLEAVRSPSFEK (M204) (SEQ ID NO:201); or a subsequence or fragment thereof. In one embodiment, the N-terminal R residue is deleted.

In certain embodiments, the peptide comprises or consists of: RDSSPLLQFGGQVRLRHLYTSGPHGLSSCFLRIRADGVVDCARGQSAHSLLEIKAVALRT VAIKGVHSVRYLCMGADGKMQGLLQYSEEDCAFEEEILEDGYNVYRSEKHRLPVSLSS AKQRQLYKNRGFLPLSHFLPMLPMVPEEPEDLRGHLESDMFSSPLETDSMDPFGLVTGL EAVRSPSFEK (M205) (SEQ ID NO:202); or a subsequence or fragment thereof. In one embodiment, the N-terminal R residue is deleted.

In some embodiments, the peptide comprises or consists of: RHPIPDSSPLLQFGGQVRLRHLYTSGPHGLSSCFLRIRADGVVDCARGQSAHSLLEIKAV ALRTVAIKGVHSVRYLCMGADGKMQGLLQYSEEDCAFEEEILEDGYNVYRSEKHRLPV SLSSAKQRQLYKNRGFLPLSHFLPMLPMVPEEPEDLRGHLESDNIFSSPLETDSMDPFGLV TGLEAVRSPSFEK (M206) (SEQ ID NO:203); or a subsequence or fragment thereof. In one embodiment, the N-terminal R residue is deleted.

In other embodiments, the peptide comprises or consists of: MRDSSPLVHYGWGDPIRLRHLYTSGPHGLSSCFLRIRADGVVDCARGQSAHSLLEIKAV ALRTVAIKGVHSVRYLCMGADGKMQGLLQYSEEDCAFEEEILEDGYNVYRSEKHRLPV SLSSAKQRQLYKNRGFLPLSHFLPMLPMVPEEPEDLRGHLESDMFSSPLETDSMDPFGLV TGLEAVRSPSFEK (M207) (SEQ ID NO:204); or a subsequence or fragment thereof.

In some embodiments, the peptide is a variant peptide designated M139. In some embodiments, the peptide comprises an amino acid sequence set forth in SEQ ID NO:193. In other embodiments, the peptide consists of an amino acid sequence set forth in SEQ ID NO:193. In some embodiments, the peptide is a variant peptide designated M140. In some embodiments, the peptide comprises an amino acid sequence set forth in SEQ ID NO:194. In other embodiments, the peptide consists of an amino acid sequence set forth in SEQ ID NO:194. In some embodiments, the peptide is a variant peptide designated M141. In some embodiments, the peptide comprises an amino acid sequence set forth in SEQ ID NO:195. In other embodiments, the peptide consists of an amino acid sequence set forth in SEQ ID NO:195. In some embodiments, the peptide is a variant peptide designated M160. In some embodiments, the peptide comprises an amino acid sequence set forth in SEQ ID NO:196. In other embodiments, the peptide consists of an amino acid sequence set forth in SEQ ID NO:196. In some embodiments, the peptide is a variant peptide designated M200. In some embodiments, the peptide comprises an amino acid sequence set forth in SEQ ID NO:197. In other embodiments, the peptide consists of an amino acid sequence set forth in SEQ ID NO:197. In some embodiments, the peptide is a variant peptide designated M201. In some embodiments, the peptide comprises an amino acid sequence set forth in SEQ ID NO:198. In other embodiments, the peptide consists of an amino acid sequence set forth in SEQ ID NO:198. In other embodiments, the peptide is a variant peptide designated M202. In some embodiments, the peptide comprises an amino acid sequence set forth in SEQ ID NO:199. In other embodiments, the peptide consists of an amino acid sequence set forth in SEQ ID NO:199. In certain embodiments, the peptide is a variant peptide designated M203. In some embodiments, the peptide comprises an amino acid sequence set forth in SEQ ID NO:200. In other embodiments, the peptide consists of an amino acid sequence set forth in SEQ ID NO:200. In some embodiments, the peptide is a variant peptide designated M204. In some embodiments, the peptide comprises an amino acid sequence set forth in SEQ ID NO:201. In other embodiments, the peptide consists of an amino acid sequence set forth in SEQ ID NO:201. In another embodiment, the peptide is a variant peptide designated M205. In some embodiments, the peptide comprises an amino acid sequence set forth in SEQ ID NO:202. In other embodiments, the peptide consists of an amino acid sequence set forth in SEQ ID NO:202. In other embodiments, the peptide is a variant peptide designated M206. In some embodiments, the peptide comprises an amino acid sequence set forth in SEQ ID NO:203. In other embodiments, the peptide consists of an amino acid sequence set forth in SEQ ID NO:203. In yet other embodiments, the peptide is a variant peptide designated M207. In some embodiments, the peptide comprises an amino acid sequence set forth in SEQ ID NO:204. In other embodiments, the peptide consists of an amino acid sequence set forth in SEQ ID NO:204.

In various additional particular aspects, the N-terminus of the peptide sequence comprises or consists of any of:

HPIPDSSPLLQFGGQVRLRHLYTSG (M5-R)(amino acids 1-25
of SEQ ID NO: 160);
DSSPLLQFGGQVRLRHLYTSG (M6-R)(amino acids 2-22 of
SEQ ID NO: 6);
RPLAFSDSSPLLQFGGQVRLRHLYTSG (M7)(amino acids 1-27
of SEQ ID NO: 7);
HPIPDSSPLLQWGDPIRLRHLYTSG (M8-R)(amino acids 2-26
of SEQ ID NO: 8);
HPIPDSSPLLQFGWGDPIRLRHLYTSG (M9-R)(amino acids 2-28
of SEQ ID NO: 9);
HPIPDSSPHVHYGWGDPIRLRHLYTSG (M10-R)(amino acids 2-28
of SEQ ID NO: 10);
RPLAFSDAGPLLQWGDPIRLRHLYTSG (M11)(amino acids 1-27
of SEQ ID NO: 11);
RPLAFSDAGPLLQFGWGDPIRLRHLYTSG (M12)(amino acids 1-
29 of SEQ ID NO: 12);
RPLAFSDAGPLLQFGGQVRLRHLYTSG (M13)(amino acids 1-27
of SEQ ID NO: 13);
HPIPDSSPHVHYGGQVRLRHLYTSG (M14-R)(amino acids 2-26
of SEQ ID NO: 14);
RPLAFSDAGPHVHYGGQVRLRHLYTSG (M15)(amino acids 1-27
of SEQ ID NO: 15);
RPLAFSDAGPHVHWGDPIRLRHLYTSG (M16)(amino acids 1-27
of SEQ ID NO: 16);
RPLAFSDAGPHVGWGDPIRLRHLYTSG (M17)(amino acids 1-27
of SEQ ID NO: 17);
RPLAFSDAGPHYGWGDPIRLRHLYTSG (M18)(amino acids 1-27
of SEQ ID NO: 18);
RPLAFSDAGPVYGWGDPIRLRHLYTSG (M19)(amino acids 1-27
of SEQ ID NO: 19);
RPLAFSDAGPVHGWGDPIRLRHLYTSG (M20)(amino acids 1-27
of SEQ ID NO: 20);
RPLAFSDAGPVHYWGDPIRLRHLYTSG (M21)(amino acids 1-27
of SEQ ID NO: 21);
RPLAFSDAGPHVHGWGDPIRLRHLYTSG (M22)(amino acids 1-
27 of SEQ ID NO: 22);
RPLAFSDAGPHHGWGDPIRLRHLYTSG (M23)(amino acids 1-27
of SEQ ID NO: 23);
RPLAFSDAGPHHYWGDPIRLRHLYTSG (M24)(amino acids 1-27
of SEQ ID NO: 24);
RPLAFSDAGPHVYWGDPIRLRHLYTSG (M25)(amino acids 1-27
of SEQ ID NO: 25);
RPLAFSDSSPLVHWGDPIRLRHLYTSG (M26)(amino acids 1-27
of SEQ ID NO: 26);
RPLAFSDSSPHVHWGDPIRLRHLYTSG (M27)(amino acids 1-27
of SEQ ID NO: 27);
RPLAFSDAGPHVWGDPIRLRHLYTSG (M28)(amino acids 1-26
of SEQ ID NO: 28);
RPLAFSDAGPHVHYWGDPIRLRHLYTSG (M29)(amino acids 1-
28 of SEQ ID NO: 29);
RPLAFSDAGPHVHYAWGDPIRLRHLYTSG (M30)(amino acids 1-
29 of SEQ ID NO: 30);
RHPIPDSSPLLQFGAQVRLRHLYTSG (M31)(amino acids 1-26
of SEQ ID NO: 31);
RHPIPDSSPLLQFGDQVRLRHLYTSG (M32)(amino acids 1-26
of SEQ ID NO: 32);
RHPIPDSSPLLQFGPQVRLRHLYTSG (M33)(amino acids 1-26
of SEQ ID NO: 33);
RHPIPDSSPLLQFGGAVRLRHLYTSG (M34)(amino acids 1-26
of SEQ ID NO: 34);
RHPIPDSSPLLQFGGEVRLRHLYTSG (M35)(amino acids 1-26
of SEQ ID NO: 35);
RHPIPDSSPLLQFGGNVRLRHLYTSG (M36)(amino acids 1-26
of SEQ ID NO: 36);
RHPIPDSSPLLQFGGQARLRHLYTSG (M37)(amino acids 1-26
of SEQ ID NO: 37);
RHPIPDSSPLLQFGGQIRLRHLYTSG (M38)(amino acids 1-26
of SEQ ID NO: 38);
RHPIPDSSPLLQFGGQTRLRHLYTSG (M39)(amino acids 1-26
of SEQ ID NO: 39);
RHPIPDSSPLLQFGWGQPVRLRHLYTSG (M40)(amino acids 1-
28 of SEQ ID NO: 40);
DAGPHVHYGWGDPIRLRHLYTSG (M74-R)(amino acids 2-24
of SEQ ID NO: 74);
VHYGWGDPIRLRHLYTSG (M75-R)(amino acids 2-19 of SEQ
ID NO: 75);
RLRHLYTSG (M77-R)(amino acids 2-10 of SEQ ID NO:
77);
RHPIPDSSPLLQFGWGDPIRLRHLYTSG (M9)(amino acids 1-28
of SEQ ID NO: 9);
RHPIPDSSPLLQWGDPIRLRHLYTSG (M8)(amino acids 1-26
of SEQ ID NO: 8);
RPLAFSDAGPLLQFGWGDPIRLRHLYTSG (M12)(amino acids 1-
29 of SEQ ID NO: 12);
RHPIPDSSPHVHYGWGDPIRLRHLYTSG (M10)(amino acids 1-
28 of SEQ ID NO: 10);
RPLAFSDAGPLLQFGGQVRLRHLYTSG (M13)(amino acids 1-27
of SEQ ID NO: 13);
RHPIPDSSPHVHYGGQVRLRHLYTSG (M14)(amino acids 1-26
of SEQ ID NO: 14);
RPLAFSDAGPHVHYGGDIRLRHLYTSG (M43)amino acids 1-27
of SEQ ID NO: 43);
or
RDSSPLLQFGGQVRLRHLYTSG (M6)(amino acids 1-22 of
SEQ ID NO: 6);

or any of the foregoing peptide sequences where the amino terminal R residue is deleted.

In certain embodiments, the peptide comprises or consists of any of:

HPIPDSSPLLQFGGQVRLRHLYTSG (M5-R)(amino acids 1-25
of SEQ ID NO: 160);
DSSPLLQFGGQVRLRHLYTSG (M6-R)(amino acids 2-22 of
SEQ ID NO: 6);
RPLAFSDSSPLLQFGGQVRLRHLYTSG (M7)(amino acids 1-27
of SEQ ID NO: 7);
HPIPDSSPLLQWGDPIRLRHLYTSG (M8-R)(amino acids 2-26
of SEQ ID NO: 8);
HPIPDSSPLLQFGWGDPIRLRHLYTSG (M9-R)(amino acids 2-
28 of SEQ ID NO: 9);
HPIPDSSPHVHYGWGDPIRLRHLYTSG (M10-R)(amino acids 2-
28 of SEQ ID NO: 10);
RPLAFSDAGPLLQWGDPIRLRHLYTSG (M11)(amino acids 1-27
of SEQ ID NO: 11);
RPLAFSDAGPLLQFGWGDPIRLRHLYTSG (M12)(amino acids 1-
29 of SEQ ID NO: 12);
RPLAFSDAGPLLQFGGQVRLRHLYTSG (M13)(amino acids 1-27
of SEQ ID NO: 13);
HPIPDSSPHVHYGGQVRLRHLYTSG (M14-R)(amino acids 2-26
of SEQ ID NO: 14);
RPLAFSDAGPHVHYGGQVRLRHLYTSG (M15)(amino acids 1-27
of SEQ ID NO: 15);
RPLAFSDAGPHVHWGDPIRLRHLYTSG (M16)(amino acids 1-27
of SEQ ID NO: 16);
RPLAFSDAGPHVGWGDPIRLRHLYTSG (M17)(amino acids 1-27
of SEQ ID NO: 17);
RPLAFSDAGPHYGWGDPIRLRHLYTSG (M18)(amino acids 1-27
of SEQ ID NO: 18);
RPLAFSDAGPVYGWGDPIRLRHLYTSG (M19)(amino acids 1-27
of SEQ ID NO: 19);
RPLAFSDAGPVHGWGDPIRLRHLYTSG (M20)(amino acids 1-27
of SEQ ID NO: 20);
RPLAFSDAGPVHYWGDPIRLRHLYTSG (M21)(amino acids 1-27
of SEQ ID NO: 21);
RPLAFSDAGPHVHGWGDPIRLRHLYTSG (M22)(amino acids 1-
27 of SEQ ID NO: 22);
RPLAFSDAGPHHGWGDPIRLRHLYTSG (M23)(amino acids 1-27
of SEQ ID NO: 23);
RPLAFSDAGPHHYWGDPIRLRHLYTSG (M24)(amino acids 1-27
of SEQ ID NO: 24);
RPLAFSDAGPHVYWGDPIRLRHLYTSG (M25)(amino acids 1-27
of SEQ ID NO: 25);
RPLAFSDSSPLVHWGDPIRLRHLYTSG (M26)(amino acids 1-27
of SEQ ID NO: 26);
RPLAFSDSSPHVHWGDPIRLRHLYTSG (M27)(amino acids 1-27
of SEQ ID NO: 27);
RPLAFSDAGPHVWGDPIRLRHLYTSG (M28)(amino acids 1-26
of SEQ ID NO: 28);
RPLAFSDAGPHVHYWGDPIRLRHLYTSG (M29)(amino acids 1-
28 of SEQ ID NO: 29);
RPLAFSDAGPHVHYAWGDPIRLRHLYTSG (M30)(amino acids 1-
29 of SEQ ID NO: 30);
RHPIPDSSPLLQFGAQVRLRHLYTSG (M31)(amino acids 1-26
of SEQ ID NO: 31);
RHPIPDSSPLLQFGDQVRLRHLYTSG (M32)(amino acids 1-26
of SEQ ID NO: 32);
RHPIPDSSPLLQFGPQVRLRHLYTSG (M33)(amino acids 1-26
of SEQ ID NO: 33);
RHPIPDSSPLLQFGGAVRLRHLYTSG (M34)(amino acids 1-26
of SEQ ID NO: 34);
RHPIPDSSPLLQFGGEVRLRHLYTSG (M35)(amino acids 1-26
of SEQ ID NO: 35);
RHPIPDSSPLLQFGGNVRLRHLYTSG (M36)(amino acids 1-26
of SEQ ID NO: 36);
RHPIPDSSPLLQFGGQARLRHLYTSG (M37)(amino acids 1-26
of SEQ ID NO: 37);
RHPIPDSSPLLQFGGQIRLRHLYTSG (M38)(amino acids 1-26
of SEQ ID NO: 38);
RHPIPDSSPLLQFGGQTRLRHLYTSG (M39)(amino acids 1-26
of SEQ ID NO: 39);
RHPIPDSSPLLQFGWGQPVRLRHLYTSG (M40)(amino acids 1-
28 of SEQ ID NO: 40);
DAGPHVHYGWGDPIRLRHLYTSG (M74-R)(amino acids 2-24
of SEQ ID NO: 74);
VHYGWGDPIRLRHLYTSG (M75-R)(amino acids 2-19 of SEQ
ID NO: 75);
RLRHLYTSG (M77-R)(amino acids 2-10 of SEQ ID NO:
77);
RHPIPDSSPLLQFGWGDPIRLRHLYTSG (M9)(amino acids 1-28
of SEQ ID NO: 9);
RHPIPDSSPLLQWGDPIRLRHLYTSG (M8)(amino acids 1-26
of SEQ ID NO: 8);
RPLAFSDAGPLLQFGWGDPIRLRHLYTSG (M12)(amino acids 1-
29 of SEQ ID NO: 12);
RHPIPDSSPHVHYGWGDPIRLRHLYTSG (M10)(amino acids 1-
28 of SEQ ID NO: 10);
RPLAFSDAGPLLQFGGQVRLRHLYTSG (M13)(amino acids 1-27
of SEQ ID NO: 13);
RHPIPDSSPHVHYGGQVRLRHLYTSG (M14)(amino acids 1-26
of SEQ ID NO: 14);
RPLAFSDAGPHVHYGGDIRLRHLYTSG (M43) amino acids 1-27
of SEQ ID NO: 43);
or
RDSSPLLQFGGQVRLRHLYTSG (M6)(amino acids 1-22 of
SEQ ID NO: 6).

In some embodiments, the peptide comprise one of the foregoing sequences. In another embodiment, the peptide consists of one of the foregoing sequences. In some embodiments, the peptide comprises a C-terminal region comprising a portion of SEQ ID NO:99 (FGF19), the C-terminal region having a first amino acid position and a last amino acid position, wherein the C-terminal region comprises amino acid residues 16-29 of SEQ ID NO:99 (FGF19), WGDPIRLRHLYTSG (SEQ ID NO:169), wherein the W residue corresponds to the first amino acid position of the C-terminal region.

In various further particular aspects, a peptide sequence comprises or consists of:

(SEQ ID NO: 160)
HPIPDSSPLLQFGGQVRLRHLYTSGPHGLSSCFLRIRADGVVDCARGQSA
HSLLEIKAVALRTVAIKGVHSVRYLCMGADGKMQGLLQYSEEDCAFEEEI
RPDGYNVYRSEKHRLPVSLSSAKQRQLYKNRGFLPLSHFLPMLPMVPEEP
EDLRGHLESDMFSSPLETDSMDPFGLVTGLEAVRSPSFEK;
(SEQ ID NO: 138 or 161)
DSSPLLQFGGQVRLRHLYTSGPHGLSSCFLRIRADGVVDCARGQSAHSLL
EIKAVALRTVAIKGVHSVRYLCMGADGKMQGLLQYSEEDCAFEEEIRPDG
YNVYRSEKHRLPVSLSSAKQRQLYKNRGFLPLSHFLPMLPMVPEEPEDLR
GHLESDMFSSPLETDSMDPFGLVTGLEAVRSPSFEK;
(SEQ ID NO: 1 or 139)
RPLAFSDASPHVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEIRPDGYNVYRSEKHRLPVSLSSAKQRQLYKNRGFLPLSHFLPMLPMV
PEEPEDLRGHLESDMFSSPLETDSMDPFGLVTGLEAVRSPSFEK;
(SEQ ID NO: 2 or 140)
RPLAFSDSSPLVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEIRPDGYNVYRSEKHRLPVSLSSAKQRQLYKNRGFLPLSHFLPMLPMV
PEEPEDLRGHLESDMFSSPLETDSMDPFGLVTGLEAVRSPSFEK;
or
(SEQ ID NO: 141)
DSSPLVHYGWGDPIRLRHLYTSGPHGLSSCFLRIRADGVVDCARGQSAHS
LLEIKAVALRTVAIKGVHSVRYLCMGADGKMQGLLQYSEEDCAFEEEIRP
DGYNVYRSEKHRLPVSLSSAKQRQLYKNRGFLPLSHFLPMLPMVPEEPED
LRGHLESDMFSSPLETDSMDPFGLVTGLEAVRSPSFEK;

or a subsequence or fragment thereof of any of the foregoing peptide sequences. In certain embodiments of any of the foregoing peptide sequences, the R terminal residue is deleted.

In further embodiments, a peptide sequence comprises or consists of: MRDSSPLVHYGWGDPIRLRHLYTSGPHGLSSCFLRIRADGVVDCARGQSAHSLLEIKAV ALRTVAIKGVHSVRYLCMGADGKMQGLLQYSEEDCAFEEEIRPDGYNVYRSEKHRLPV SLSSAKQRQLYKNRGFLPLSHFLPMLPMVPEEPEDLRGHLESDMFSSPLETDSMDPFGLV TGLEAVRSPSFEK (M70) (SEQ ID NO:70), or a subsequence or fragment thereof. In certain embodiments, a peptide sequence includes the addition of amino acid residues 30-194 of SEQ ID NO:99 (FGF19) at the C-terminus, resulting in a chimeric polypeptide.

In some embodiments, a peptide sequence has at least one amino acid substitution to amino acid residues 125-129 of SEQ ID NO:99 (FGF19), EIRPD. In other embodiments, the peptide sequence has at least one amino acid substitution to amino acid residues 126-128 of SEQ ID NO:99 (FGF19), IRP. In other embodiments, the peptide sequence has at least one amino acid substitution to amino acid residues 127-128 of SEQ ID NO:99 (FGF19), RP. In other embodiments, the peptide sequence has at least one amino acid substitution to amino acid residues 1-124 of SEQ ID NO:99 (FGF19) and/or to amino acid residues 130-194 of SEQ ID NO:99 (FGF19). For example, in certain embodiments, a peptide sequence comprises substitution to one of amino acid residues 127-128 of SEQ ID NO:99 (FGF19), RP, wherein at least one amino acid substitution is R127L or P128E. Said substitutions within a corresponding FGF19 sequence (e.g., EIRPD, IRP or RP) of a peptide variant provided herein is also contemplated. In certain embodiments, the peptide comprises both a R127L and P128E substitution to amino acid residues 127-128 of SEQ ID NO:99 (FGF19), RP, or the corresponding FGF19 sequence thereof in a variant peptide provided herein. In certain embodiments, the amino acid sequence of the peptide comprises at least one amino acid substitution in the Loop-8 region of FGF19, or the corresponding FGF19 sequence thereof in a variant peptide provided herein. In certain embodiments, the amino acid sequence of the peptide comprises one amino acid substitution to the EIRPD (amino acids 2-6 of SEQ ID NO:190) amino acid sequence in the Loop-8 region of FGF19. In some embodiments, the amino acid sequence of the peptide comprises two amino acid substitutions to the EIRPD (amino acids 2-6 of SEQ ID NO:190) amino acid sequence in the Loop-8 region of FGF19. In other embodiments, the amino acid sequence of the peptide comprises three amino acid substitutions to the EIRPD (amino acids 2-6 of SEQ ID NO:190) amino acid sequence in the Loop-8 region of FGF19. In certain embodiments, the amino acid sequence of the peptide comprises four amino acid substitutions to the EIRPD (amino acids 2-6 of SEQ ID NO:190) amino acid sequence in the Loop-8 region of FGF19. In some embodiments, the amino acid sequence of the peptide comprises five amino acid substitutions to the EIRPD (amino acids 2-6 of SEQ ID NO:190) amino acid sequence in the Loop-8 region of FGF19. In certain embodiments, the amino acid sequence of the peptide comprises one amino acid substitution to the IRP (amino acids 3-5 of SEQ ID NO:190) amino acid sequence in the Loop-8 region of FGF19. In some embodiments, the amino acid sequence of the peptide comprises two amino acid substitutions to the IRP (amino acids 3-5 of SEQ ID NO:190) amino acid sequence in the Loop-8 region of FGF19. In other embodiments, the amino acid sequence of the peptide comprises three amino acid substitutions to the IRP (amino acids 3-5 of SEQ ID NO:190) amino acid sequence in the Loop-8 region of FGF19. In certain embodiments, the amino acid sequence of the peptide comprises one amino acid substitution to the RP (amino acids 4-5 of SEQ ID NO:190) amino acid sequence in the Loop-8 region of FGF19. In some embodiments, the amino acid sequence of the peptide comprises two amino acid substitutions to the RP (amino acids 4-5 of SEQ ID NO:190) amino acid sequence in the Loop-8 region of FGF19. In certain embodiments, the amino acid substitution to the RP (amino acids 4-5 of SEQ ID NO:190) amino acid sequence in the Loop-8 region of FGF19 is an Arg (R) to Leu (L) substitution. In other embodiments, the substitution to the RP (amino acids 4-5 of SEQ ID NO:190) amino acid sequence in the Loop-8 region of FGF19 is a Pro (P) to Glu (E) substitution. In some embodiments, the substitutions to the RP (amino acids 4-5 of SEQ ID NO:190) amino acid sequence in the Loop-8 region of FGF19 is an Arg (R) to Leu (L) substitution and a Pro (P) to Glu (E) substitution. In specific embodiments, the foregoing substitution(s) in the Loop-8 region of FGF19 is in the corresponding FGF19 sequence thereof in a variant peptide provided herein. That is, said substitutions within a corresponding FGF19 sequence (e.g., EIRPD, IRP or RP) of a peptide variant provided herein is also contemplated.

Peptide or chimeric sequences provided herein can be of any suitable length. In particular embodiments, the N-terminal or C-terminal region of the peptide or chimeric sequence is from about 20 to about 200 amino acid residues in length. In further particular embodiments, a chimeric peptide sequence or peptide sequence has at least one amino acid deletion. In other particular aspects, a peptide or chimeric sequence has 1, 2, 3, 4, 5, 6, 7, 8, 9, 10, 11, 12, 13, 14, 15, 16, 17, 18, 19, 20 or more amino acid deletions from the amino terminus, the carboxy-terminus or internally. In one embodiment, the amino acid substitution, or deletion is at any of amino acid positions 8-20 of FGF19 (AGPHVHYGWGDPI) (SEQ ID NO:187). In further particular embodiments, a peptide or chimeric sequence has an N-terminal region, or a C-terminal region that comprises or consists of an amino acid sequence of about 5 to 10, 10 to 20, 20 to 30, 30 to 40, 40 to 50, 60 to 70, 70 to 80, 80 to 90, 90 to 100 or more amino acids. In additional more particular embodiments, a peptide or chimeric sequence has a FGF19 sequence portion, or a FGF21 sequence portion that comprises or consists of an amino acid sequence of about 5 to 10, 10 to 20, 20 to 30, 30 to 40, 40 to 50, 50 to 60, 60 to 70, 70 to 80, 80 to 90, 90 to 100 or more amino acids of FGF19 or FGF21.

In various further embodiments, a peptide or chimeric sequence has an amino acid substitution, an addition, insertion or is a subsequence that has at least one amino acid deleted. Such amino acid substitutions, additions, insertions and deletions of a peptide sequence can be 1, 2, 3, 4, 5, 6, 7, 8, 9, 10 or more amino acid residues (10-20, 20-30, 30-40, 40-50, etc.), for example, at the N- or C-terminus, or internal. For example, a subsequence that has 1, 2, 3, 4, 5, 6, 7, 8, 9, 10, 11, 12, 13, 14, 15, 16, 17, 18, 19, 20 or more amino acid deletions from the amino terminus, the carboxy-terminus or internally. In a particular aspect, the amino acid substitution, or deletion is at any of amino acid positions 8-20 of FGF19 (AGPHVHYGWGDPI) (SEQ ID NO:187).

In still more particular aspects, a peptide or chimeric sequence includes all or a portion of a FGF19 sequence set forth as:

(SEQ ID NO: 188)
PHGLSSCFLRIRADGVVDCARGQSAHSLLEIKAVALRTVAIKGVHSVRYL
CMGADGKMQGLLQYSEEDCAFEEEIRPDGYNVYRSEKHRLPVSLSSAKQR
QLYKNRGFLPLSHFLPMLPMVPEEPEDLRGHLESDMFSSPLETDSMDPFG
LVTGLEAVRSPSFEK

positioned at the C-terminus of the peptide, or the amino terminal “R” residue is deleted from the sequence.

In various embodiments, a peptide or chimeric sequence has a function or activity greater or less than a comparison sequence. In further particular embodiments, chimeric peptide sequences and peptide sequences have particular functions or activities. In one aspect, a chimeric peptide sequence or peptide sequence maintains or increases a fibroblast growth factor receptor 4 (FGFR4) mediated activity. In additional aspects, a chimeric peptide sequence or peptide sequence binds to FGFR4 or activates FGFR4, or does not detectably bind to FGFR4 or activate FGFR4, or binds to FGFR4 with an affinity less than, comparable to or greater than FGF19 binding affinity for FGFR4, or activates FGFR4 to an extent or amount less than, comparable to or greater than FGF19 activates FGFR4. In some embodiments, a chimeric peptide sequence or peptide sequence provided herein activates FGFR4 to an extent or amount less than the extent or amount that FGF19 activates FGFR4. In some embodiments, a chimeric peptide sequence or peptide sequence provided herein activates FGFR4 to an extent or amount comparable to the extent or amount that FGF19 activates FGFR4. In some embodiments, a chimeric peptide sequence or peptide sequence provided herein activates FGFR4 to an extent or amount greater than the extent or amount that FGF19 activates FGFR4.

In one embodiment, a chimeric peptide sequence or peptide sequence provided herein maintains a FGFR4 mediated activity. In one embodiment, a chimeric peptide sequence or peptide sequence provided herein increases a FGFR4 mediated activity. In some embodiments, a chimeric peptide sequence or peptide sequence provided herein binds to FGFR4 with an affinity less than FGF19 binding affinity for FGFR4. In some embodiments, a chimeric peptide sequence or peptide sequence provided herein binds to FGFR4 with an affinity comparable to FGF19 binding affinity for FGFR4. In some embodiments, a chimeric peptide sequence or peptide sequence provided herein binds to FGFR4 with an affinity greater than FGF19 binding affinity for FGFR4. In some embodiments, a chimeric peptide sequence or peptide sequence provided herein does not detectably bind to FGFR4.

In further aspects, a chimeric peptide sequence or peptide sequence has reduced HCC formation compared to FGF19, or a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the WGDPI (SEQ ID NO:170) sequence at amino acids 16-20 of FGF19; or has greater glucose lowering activity compared to FGF19, or a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the WGDPI (SEQ ID NO:170) sequence at amino acids 16-20 of FGF19; has less lipid increasing activity compared to FGF19, or a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the WGDPI (SEQ ID NO:170) sequence at amino acids 16-20 of FGF19; or has less triglyceride, cholesterol, non-HDL or HDL increasing activity compared to FGF19, or a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the WGDPI (SEQ ID NO:170) sequence at amino acids 16-20 of FGF19; or the peptide sequence has less lean mass reducing activity compared to FGF21. Such functions and activities can be ascertained in vitro or in vivo, for example, in a db/db mouse.

In one embodiment, a peptide or chimeric sequence has a function or activity greater or less than a comparison sequence. In some embodiments, the comparison sequence is FGF19. In another embodiment, the comparison sequence is FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the WGDPI (SEQ ID NO:170) sequence at amino acids 16-20 of FGF19. In one embodiment, a peptide or chimeric peptide sequence provided herein has greater glucose lowering activity compared to a comparison sequence. In another embodiment, a peptide or chimeric peptide sequence provided herein has less lipid increasing activity compared to a comparison sequence. In other embodiment, a peptide or chimeric peptide sequence provided herein has lower or reduced lipid (e.g., triglyceride, cholesterol, non-HDL) activity compared to a comparison sequence. In other embodiments, a peptide or chimeric peptide sequence provided herein has more HDL increasing activity as compared to a comparison sequence. In other embodiment, a peptide or chimeric peptide sequence provided herein has less lean mass reducing activity compared to a comparison sequence or FGF21.

In further additional various embodiments, a peptide or chimeric sequence includes one or more L-amino acids, D-amino acids, non-naturally occurring amino acids, or amino acid mimetic, derivative or analogue. In still further various embodiments, a peptide or chimeric sequence has an N-terminal region, or a C-terminal region, or a FGF19 sequence portion, or a FGF21 sequence portion, joined by a linker or spacer.

In some embodiments, the methods provided herein further include administering an additional agent to the subject. The additional agent can be, for example, a bulk-forming agent, an emollient stool softener, a lubricant, a prokinetic, a laxative, an osmotic agents, or prosecretory drug.

In some embodiments, the subject is an animal. In some embodiments, the subject is a human. In some embodiments, the subject has a gastrointestinal motility-related disorder. In some embodiments, the subject has constipation. In some embodiments, the constipation is functional constipation. In some embodiments, the constipation is caused by medication. In some embodiments, the constipation is induced by, or a side effect of, another disease. In some embodiments, the subject has chronic idiopathic constipation. In some embodiments, the subject has a disease associated with constipation. In some embodiments, the subject does not have a gastrointestinal motility-related disorder but is at risk of developing a gastrointestinal motility-related disorder. In some embodiments, the subject does not have constipation or a disorder known to cause constipation but is at risk of developing constipation or the disorder. In some embodiments, the subject has irritable bowel syndrome (IBS). In some embodiments, the subject has gastroparesis. In some embodiments, the subject has constipation-predominant IBS. In some embodiments, the subject has a metabolic disorder. In some embodiments, the subject has an endocrine disorder. In some embodiments, the subject has gastroesophageal reflux disease. In some embodiments, the subject has intestinal dysmotility. In some embodiments, the subject has achalasia. In some embodiments, the subject has scleroderma. In some embodiments, the subject has hypercalcemia, hypothyroidism, hyperparathyroidism, porphyria, chronic kidney disease, pan-hypopituitarism, diabetes mellitus, cystic fibrosis, or celiac disease. In some embodiments, the subject has a glucose disorder. In some embodiments, the subject has a cholesterol or triglyceride metabolism disorder. In some embodiments, the subject has diabetes mellitus. In some embodiments, the subject has Type I diabetes. In some embodiments, the subject has Type II diabetes. In some embodiments, the subject has a neurological disorder. In some embodiments, the subject has anismus, descending perineum syndrome, or Hirschsprung's disease. In some embodiments, the subject has colorectal cancer, strictures, rectocoles, post-surgical changes or other reasons that create space-occupying lesions within the colon. In some embodiments, the subject has gastric outlet obstruction. In some embodiments, the subject has muscular and myotonic dystrophy.

In some embodiments, the subject has a bile acid related disorder. In some embodiments, the bile acid related disorder can be metabolic syndrome; a lipid or glucose disorder; cholesterol or triglyceride metabolism; type 2 diabetes; cholestasis, intrahepatic cholestasis, primary biliary cirrhosis (PBC), primary familial intrahepatic cholestasis (PFIC), progressive PFIC, primary sclerosing choangitis (PSC), pregnancy intrahepatic cholestasis (PIC), neonatal cholestasis, and drug induced cholestasis, diseases of extrahepatic cholestasis, bile cut compression from tumor, bile duct blockade by gall stones, bile acid malabsorption and other disorders involving the distal small intestine, ileal resection, inflammatory bowel diseases, Crohn's disease, ulcerative colitis, idiopathic disorders impairing absorption of bile acids, diarrhea, bile acid diarrhea (BAD), GI symptoms, GI cancers, liver cancers, biliary cancers, colon cancer, hepatocellular cancer, bile acid synthesis abnormalities, non-alcoholic steatohepatitis (NASH), cirrhosis, portal hypertension, or any combination thereof.

In some embodiments, methods of treating a subject having a gastrointestinal motility-related disorder, with peptide sequences as described herein can ameliorate a the gastrointestinal motility-related disorder by stimulating bowel function in the subject. In some embodiments, methods of treating a subject having constipation, or in some specific embodiments, functional constipation, with peptide sequences as described herein can ameliorate constipation by stimulating bowel function in the subject. In some embodiments, the methods described herein accelerate colonic transit (“CT”) in the subject. In some embodiments, the methods described herein increases stool frequency in the subject. In some embodiments, the methods described herein improves stool consistency in the subject. In some embodiments, the methods described herein improves ease of passage in the subject. In some embodiments, the methods described herein accelerates gastric emptying in the subject. In some embodiments, the methods described herein accelerates ascending colon empting in the subject. In some embodiments, the methods described herein reduces fecal fat in the subject. In some embodiments, the methods described herein reduces fecal bile acids in the subject. In some embodiments, the methods described herein increases the percentage of primary bile acids in total fecal bile acids in the subject. In some embodiments, the methods described herein decrease hepatic bile acid synthesis in the subject. In some embodiments, the methods described herein stimulate excitatory neural control of colonic mobility in the subject.

In some embodiments, provided herein are also methods for predicting the responsiveness of a subject having a gastrointestinal motility-related disorder to the treatment with a peptide sequence described herein, wherein the methods comprise genotyping the subject, wherein the subject who is a carrier of KLB minor allele rs17618244 is determined to have a greater response to the treatment than a non-carrier. In some embodiments, provided herein are also methods to select a subject having a gastrointestinal motility-related disorder for the treatment with a peptide sequence described herein, comprising genotyping the subject, wherein the subject who is a carrier of KLB minor allele rs17618244 is selected for the treatment. The peptide sequence can be any peptide sequence exemplified in Table 1 or otherwise described in this application. In some embodiments, the peptide sequence is M69. In some embodiments the peptide sequence is M70. In some embodiments, the peptide sequence is fused with a Fc region. In some embodiments, the peptide sequence is M69 fused a human antibody Fc fragment. In some embodiments, the peptide sequence is M70 fused a human antibody Fc fragment.

In some embodiments, provided herein are methods of treating a subject having a gastrointestinal motility-related disorder comprising first genotyping the subject to determine the presence of the KLB minor allele rs17618244, and administering a therapeutically effective amount of a peptide sequence described herein to the subject determined to be a carrier of the KLB minor allele rs17618244. In some embodiments, genotyping a subject can be performed using Single Nucleotide Polymorphism (SNP) assay, restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, DNA microarrays, Mass Spectrometry (MS), or denaturing high-performance liquid chromatography (DHPLC). Methods of genotyping also include whole genome sequencing.

In some embodiments, provided herein are also methods of treating a subject having a gastrointestinal motility-related disorder comprising (A) providing a sample from the subject; (B) selecting the subject for treatment based on the presence of the KLB minor allele rs17618244 in the sample; and (C) administering a therapeutically effective amount of a peptide sequence described herein to the subject.

In some embodiments, provided herein are also methods of treating a subject having a gastrointestinal motility-related disorder comprising (A) obtaining a sample from the subject; (B) detecting the presence of the KLB minor allele rs17618244 in the sample; and (C) diagnosing the subject as highly sensitive to a peptide sequence if the KLB minor allele rs17618244 is present in the sample; and (D) administering a therapeutically effective amount of the peptide sequence described herein to the subject.

In some embodiments, provided herein are also methods of treating a subject having a gastrointestinal motility-related disorder comprising (A) obtaining a sample from the subject; (B) detecting the presence of the KLB minor allele rs17618244 in the sample; (C) selecting the subject as highly sensitive to a peptide sequence if the KLB minor allele rs17618244 is present in the sample; and (D) administering a therapeutically effective amount of the peptide sequence described herein to the subject.

In some embodiments, provided herein are also methods determining the responsiveness of a subject having a gastrointestinal motility-related disorder to a treatment, comprising (A) obtaining a sample from the subject; (B) determining the presence of the KLB minor allele rs17618244 in the sample; and (C) diagnosing the subject as highly sensitive to the treatment if the KLB minor allele rs17618244 is present in the sample; wherein the treatment comprises administering a therapeutically effective amount of a peptide sequence described herein to the subject.

In some embodiments, determining the presence of the KLB minor allele rs17618244 includes detecting the presence of a complex or the formation of a reaction product in a sample from the subject having a gastrointestinal motility-related disorder, wherein the complex or reaction product indicates the presence of the KLB minor allele rs17618244. In some embodiments, the complex is detectably labeled. In some embodiments, the reaction product is detectably labeled. In some embodiments, the complex is a hybridization complex. In some embodiments, the hybridization complex is attached to a solid support.

In some embodiments, provided herein are also methods of identifying a subject having a gastrointestinal motility-related disorder as highly sensitive to a treatment, comprising (A) detecting the presence of a complex or the formation of a reaction product in a sample from the subject, wherein the complex or reaction product indicates the presence of the KLB minor allele rs17618244 in the sample; and (B) diagnosing the subject as highly sensitive to the based on the presence of the complex or reaction product in the sample; wherein the treatment comprises administering a therapeutically effective amount of a peptide sequence described herein to the subject.

In some embodiments, provided herein are also methods of predicting the responsiveness of a subject having a gastrointestinal motility-related disorder to a treatment, comprising detecting a complex or the formation of a reaction product in a sample from the subject, wherein the presence of the complex or reaction product indicates the presence of KLB minor allele rs17618244, and wherein the subject is predicted to have a greater response to the treatment than a non-carrier of KLB minor allele rs17618244; wherein the treatment comprises administering a therapeutically effective amount of a peptide sequence described herein to the subject.

In some embodiments, provided herein are also methods of determining the responsiveness of a subject having a gastrointestinal motility-related disorder to a treatment, comprising (A) obtaining a sample from the subject; (B) determining the presence of the KLB minor allele rs17618244 in the sample by detecting the presence of a complex or the formation of a reaction product, wherein the complex or reaction product indicates the presence of the KLB minor allele rs17618244 in the sample; and (C) diagnosing the subject as highly sensitive to the treatment if the KLB minor allele rs17618244 is present in the sample; wherein the treatment comprises administering a therapeutically effective amount of a peptide sequence to the subject; wherein the treatment comprises administering a therapeutically effective amount of a peptide sequence described herein to the subject.

In some embodiments, the subject who is a carrier of the KLB minor allele rs17618244 is heterozygous for KLB minor allele rs17618244. In some embodiments, the subject who is a carrier of the KLB minor allele rs17618244 is homozygous for KLB minor allele rs17618244.

The peptide sequence can be any peptide sequence exemplified in Table 1, Sequence Listing, or otherwise described in this application. In some embodiments, the peptide sequence is M69. In some embodiments the peptide sequence is M70. In some embodiments, the peptide sequence is fused with a Fc region. In some embodiments, the peptide sequence is M69 fused a human antibody Fc fragment. In some embodiments, the peptide sequence is M70 fused a human antibody Fc fragment.

In some embodiments, provided herein are also kits for predicting the responsiveness of a subject to a treatment for a gastrointestinal motility-related disorder using a peptide sequence disclosed herein, comprising at least one agent for determining the presence or absence of KLB minor allele rs17618244. In some embodiments, the kits provided herein can include an ancillary agent.

### DESCRIPTION OF DRAWINGS

FIG. 1 shows the timeline of the parallel-group, placebo-controlled, randomized, double-blind study of the effects of M70 on colonic transit, stool frequency and consistency, fecal fat and bile acids (serum and fecal) in patients with functional constipation. Timeline for both the baseline off treatment and during treatment is shown.

FIG. 2 depicts a flow-chart of method of assessing eligibility of subjects for the study.

FIG. 3A shows the results of the parallel-group, placebo-controlled, randomized, double-blind study including (i) number of bowel movement per week (BM/week); (ii) Ease of passage; (iii) ascending colon emptying half time (AC T1/2); and (iv) percentage of the primary bile acids, also known as main 1° bile acids, which are cholic acid and chenodeoxycholic acid (% CA+CDCA) in total bile acids; for all three groups of subjects, receiving either placebo, 1 mg of M70, or 6 mg of M70. As shown, M70 increased bowel movement frequency. improved ease of passage, accelerated ascending colon and increased proportion of primary bile acids.

FIG. 3B shows the results of (i) colonic transit measured as colonic geometric centre (GC24; GC48); (ii) stool consistency measured using the Bristol Stool Form Scale (BSFS); and (iii) total amount of fecal bile acids (Fecal BA mmol/48 h) for all three groups of subjects, receiving either placebo, 1 mg of M70, or 6 mg of M70. As shown, M70 increased overall colonic transit, loosened stool consistency, and reduced total fecal bile acid excretion.

FIG. 4 shows the results of colonic transit measured as colonic geometric centre (GC24) in participants with the Klothoβ (KLB) rs17618244 A (minor) allele (KLB Gln728) compared to KLB major (G) allele (KLB Arg728) in response to either placebo, 1 mg of M70, or 6 mg of M70. As shown, 6 mg of M70 greatly increased overall colonic transit in participants with KLB minor allele as compared to those with the KLB major allele.

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

### Definitions

The terms “treat”, “treating”, treatment” and the like refer to a course of action (such as administering a polypeptide or a pharmaceutical composition comprising a polypeptide) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (i.e., arresting the development or further development of the disease, disorder or condition or clinical symptoms association therewith) an active disease.

The term “in need of treatment” as used herein refers to a judgment made by a physician or other medical professional that a subject requires or will benefit from treatment.

The terms “prevent”, “preventing”, “prevention” and the like refer to a course of action (such as administering a polypeptide or a pharmaceutical composition comprising a polypeptide) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other medical professional that a subject requires or will benefit from preventative care.

The term “administer” or “administration” refers to the act of injecting or otherwise physically delivering a substance as it exists outside the body into a patient, such as by mucosal, intradermal, intravenous, intramuscular delivery and/or any other method of physical delivery described herein or known in the art. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

The term “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

The term “responsiveness” or “responsive” when used in reference to a treatment refers to the degree of effectiveness of the treatment in lessening or decreasing the symptoms of a disease, e.g., gastrointestinal-motility related disorder, being treated. For example, the term “increased responsiveness” when used in reference to a treatment of a subject refers to an increase in the effectiveness in lessening or decreasing the symptoms of the disease compared to a reference treatment (e.g., of the same subject, or of a different subject) when measured using any methods known in the art. In certain embodiments, the increase in the effectiveness is at least about 5%, at least about 10%, at least about 20%, at least about 30%, at least about 40%, or at least about 50%.

The term “metabolic syndrome” refers to an associated cluster of traits that includes, but is not limited to, hyperinsulinemia, abnormal glucose tolerance, obesity, redistribution of fat to the abdominal or upper body compartment, hypertension, dysfibrinolysis, and dyslipidemia characterized by high triglycerides, low high density lipoprotein (HDL)-cholesterol, and high small dense low density lipoprotein (LDL) particles. Subjects having metabolic syndrome are at risk for development of type 2 diabetes and/or other disorders (e.g., atherosclerosis).

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like. It will be appreciated that throughout this disclosure reference is made to amino acids according to the single letter or three letter codes. The amino acids forming all or a part of a peptide may be from among the known 21 naturally occurring amino acids, which are referred to by both their single letter abbreviations and their common three-letter abbreviation. In the peptide sequences provided herein, conventional amino acid residues have their conventional meaning. Thus, “Leu” is leucine, “Ile” is isoleucine, “Nle” is norleucine, and so on. To assist the reader, conventional amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

As used herein, the term “variant” encompasses naturally-occurring variants (e.g., homologs and allelic variants) and non-naturally-occurring variants (e.g., muteins). Naturally-occurring variants include homologs, i.e., nucleic acids and polypeptides that differ in nucleotide or amino acid sequence, respectively, from one species to another. Naturally-occurring variants include allelic variants, i.e., nucleic acids and polypeptides that differ in nucleotide or amino acid sequence, respectively, from one individual to another within a species. Non-naturally-occurring variants include nucleic acids and polypeptides that comprise a change in nucleotide or amino acid sequence, respectively, where the change in sequence is artificially introduced, e.g., the change is generated in the laboratory or other facility by human intervention (“hand of man”).

The term “native”, in reference to FGF19, refers to biologically active, naturally-occurring FGF19, including biologically active, naturally-occurring FGF19 variants. The term includes the 194 amino acid human FGF19 mature sequence.

The terms “label”, “labeling” and the like, when use in the context of a polypeptide or nucleic acid (or antibody, as appropriate) of the present disclosure are meant to refer broadly to any means useful in, for example, polypeptide purification, identification, isolation and synthesis. Labels are generally covalently bound to the polypeptide of interest and can be introduced in any manner known in the art, including attachment to a mature polypeptide (generally at the N- or C-terminus), incorporation during solid-phase peptide synthesis, or through recombinant means. Examples include, but are not limited to, fluorescence, biotinylation, and radioactive isotopes. Polypeptide and nucleic acid molecules can be labeled by both in vitro and in vivo methods. Labeling reagents and kits can be obtained from a number of commercial sources (e.g., Thermo Fischer Scientific, Rockford, Ill.; and Molecular Probes/Life Technologies; Grand Island, N.Y.).

The term “muteins” as used herein refers broadly to mutated recombinant proteins, i.e., a polypeptide comprising an artificially introduced change in amino acid sequence, e.g., a change in amino acid sequence generated in the laboratory or other facility by human intervention (“hand of man”). These proteins usually carry single or multiple amino acid substitutions and are frequently derived from cloned genes that have been subjected to site-directed or random mutagenesis, or from completely synthetic genes.

As used herein in reference to native human FGF19 or a FGF19 mutein, the terms “modified”, “modification” and the like refer to one or more changes that enhance a desired property of human FGF19, a naturally-occurring FGF19 variant, or a FGF19 mutein, wherein the change(s) does not alter the primary amino acid sequence of the FGF19. Such desired properties include, for example, enhancing solubility, prolonging the circulation half-life, increasing the stability, reducing the clearance, altering the immunogenicity or allergenicity, improving aspects of manufacturability (e.g., cost and efficiency), and enabling the raising of particular antibodies (e.g., by introduction of unique epitopes) for use in detection assays. Changes to human FGF19, a naturally-occurring FGF19 variant, or a FGF19 mutein that can be carried out include, but are not limited to, pegylation (covalent attachment of one or more molecules of polyethylene glycol (PEG), or derivatives thereof); glycosylation (e.g., N-glycosylation), polysialylation and hesylation; albumin fusion; albumin binding through, for example, a conjugated fatty acid chain (acylation); Fc-fusion; and fusion with a PEG mimetic. Some particular embodiments entail modifications involving polyethylene glycol, other particular embodiments entail modifications involving albumin, and still other particular modifications entail modifications involving glycosylation.

The terms “DNA”, “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

The term “probe” refers to a fragment of DNA or RNA corresponding to a gene or sequence of interest, wherein the fragment has been labeled radioactively (e.g., by incorporating ³²P or ³⁵S) or with some other detectable molecule, such as biotin, digoxygen or fluorescein. As stretches of DNA or RNA with complementary sequences will hybridize, a probe can be used, for example, to label viral plaques, bacterial colonies or bands on a gel that contain the gene of interest. A probe can be cloned DNA or it can be a synthetic DNA strand; the latter can be used to obtain a cDNA or genomic clone from an isolated protein by, for example, microsequencing a portion of the protein, deducing the nucleic acid sequence encoding the protein, synthesizing an oligonucleotide carrying that sequence, radiolabeling the sequence and using it as a probe to screen a cDNA library or a genomic library.

The term “heterologous” refers to two components that are defined by structures derived from different sources. For example, in the context of a polypeptide, a “heterologous” polypeptide can include operably linked amino acid sequences that are derived from different polypeptides. Similarly, in the context of a polynucleotide encoding a chimeric polypeptide, a “heterologous” polynucleotide can include operably linked nucleic acid sequences that can be derived from different genes. Exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which can be of different genetic origin than the promoter, the coding sequence or both). In the context of recombinant cells, “heterologous” can refer to the presence of a nucleic acid (or gene product, such as a polypeptide) that is of a different genetic origin than the host cell in which it is present.

The term “operably linked” refers to linkage between molecules to provide a desired function. For example, “operably linked” in the context of nucleic acids refers to a functional linkage between nucleic acid sequences. By way of example, a nucleic acid expression control sequence (such as a promoter, signal sequence, or array of transcription factor binding sites) can be operably linked to a second polynucleotide, wherein the expression control sequence affects transcription and/or translation of the second polynucleotide. In the context of a polypeptide, “operably linked” refers to a functional linkage between amino acid sequences (e.g., different domains) to provide for a described activity of the polypeptide.

As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Derived from”, in the context of an amino acid sequence or polynucleotide sequence (e.g., an amino acid sequence “derived from” a FGF19 polypeptide), is meant to indicate that the polypeptide or nucleic acid has a sequence that is based on that of a reference polypeptide or nucleic acid (e.g., a naturally occurring FGF19 polypeptide or a FGF19-encoding nucleic acid), and is not meant to be limiting as to the source or method in which the protein or nucleic acid is made. By way of example, the term “derived from” includes homologues or variants of reference amino acid or DNA sequences.

In the context of a polypeptide, the term “isolated” refers to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates the polypeptide has been separated from an environment in which it was made by either synthetic or recombinant means.

“Enriched” means that a sample is non-naturally manipulated (e.g., by a scientist or a clinician) so that a polypeptide of interest is present in a) a greater concentration (e.g., at least 3-fold greater, at least 4-fold greater, at least 8-fold greater, at least 64-fold greater, or more) than the concentration of the polypeptide in the starting sample, such as a biological sample (e.g., a sample in which the polypeptide naturally occurs or in which it is present after administration), or b) a concentration greater than the environment in which the polypeptide was made (e.g., as in a bacterial cell).

The terms “measuring” or “assaying” and grammatical variations thereof are used interchangeably herein and refer to either qualitative or quantitative determinations, or both qualitative and quantitative determinations. When the terms are used in reference to detection, any means of assessing the relative amount is contemplated, including the various methods set forth herein and known in the art. For example, gene expression can be assayed or measured by a Northern blot, Western blot, immunoprecipitation assay, or by measuring activity, function or amount of the expressed protein.

The term “predict” generally means to determine or tell in advance. When used to “predict” the effectiveness of a treatment, for example, the term “predict” can mean that the likelihood of the outcome of the treatment can be determined at the outset, before the treatment has begun, or before the treatment period has progressed substantially.

The term “sensitive” as used herein refers to the responsiveness of a subject having a disease, disorder or condition to the treatment with a drug. The term “highly sensitive” means that the subject is more sensitive to the treatment than the average population. For example, in the context of the methods disclosed herein, a subject having a gastrointestinal motility-related disorder is “sensitive” to the treatment with a treatment with peptide described herein means if the subject is responsive to the treatment. A subject who is “highly sensitive” to the treatment means that the subject is more responsive to the treatment than the average population. As described herein, subjects who are carriers of the KLB minor allele rs17618244 are highly sensitive to a treatment of the peptide described herein because they are more responsive than the non-carriers and the average population.

The phrase “single nucleotide polymorphism” or “SNP” refers to a DNA sequence variation occurring when a single nucleotide—A, T, C, or G—in the genome (or other shared sequence) differs between members of a species or paired chromosomes in an individual. For example, two sequenced DNA fragments from different individuals, AAGCCTA to AAGCTTA, contain a difference in a single nucleotide. In this case it is understood that there are two alleles: C and T. Almost all common SNPs have only two alleles. Single nucleotide polymorphisms may fall within coding sequences of genes, non-coding regions of genes, or in the intergenic regions between genes. SNPs that are not in protein-coding regions may still have consequences for gene splicing, transcription factor binding, or the sequence of non-coding RNA. There are variations between human populations, so a SNP allele that is common in one geographical or ethnic group may be much rarer, or even absent, in another. As there are for genes, there are also bioinformatics databases for SNPs. dbSNP is a SNP database from National Center for Biotechnology Information (NCBI). For example, the gene Klothoβ (KLB) has a coding SNP that results in an amino acid variation at residue 728, commonly designated as rs17618244 (Arg728G1n). The SNPs described herein by their Reference SNP Cluster Report are well known to those skilled in the art. Detailed descriptions of the SNPs described herein can be found in, for example, the Single Nucleotide Polymorphism Database (dbSNP), available at the NCBI website www.ncbi.nlm.nih.gov/projects/SNP/.

The term “major allele” when used in reference to a SNP refers to the nucleotide residue for a given SNP that is most common in a population of individuals, as distinguished from the less common allele (i.e. “minor allele”) or rare variant. For example, if there are 3 alleles, with frequencies of 0.50 for a “G”, 0.49 for a “C” and 0.01 for a “T” in a given population, the major allele will be the “G” allele with 0.50, the minor allele will be the “C” allele with 0.49 and the rare variant will be the “T” allele with 0.01. In the case of KLB rs17618244, the major allele gives rise to Arg728; and the minor allele gives rise to Gln728.

As used herein, the term “carrier” when used in connection with an allele of a gene refers to a subject whose genome includes at least one copy of the specific allele. For example, a carrier of the KLB minor allele rs17618244 refers to a subject whose genome includes at least one copy of the minor allele rs17618244(G1n728); a carrier of major allele rs17618244 refers to a subject whose genome includes at least one copy of the major allele rs17618244 (Arg728). A subject can be a carrier of the major allele rs17618244, the minor allele rs17618244, or both.

The term “heterozygous” refers to a pair of alleles that code for the same gene or trait, wherein one allele is different than the other allele. For example, a heterozygous carrier of the KLB minor allele rs17618244 carries both the major allele and the minor allele of rs17618244.

The term “homozygous” refers to a pair of alleles that code for the same gene or trait, wherein the pair of alleles are the same. For example, a homozygous carrier of the KLB minor allele rs17618244 carries two copies of the minor allele of rs17618244, but not the major allele.

The term “genotyping” refers to the process of determining differences in the genetic make-up (genotype) of an individual by examining the individual's DNA sequence using biological assays and comparing it to another individual's sequence or a reference sequence. There are well-known standard techniques used in the art for genotyping a subject. Current methods of genotyping include Single Nucleotide Polymorphism (SNP) assay, restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, DNA microarrays, Mass Spectrometry (MS), and denaturing high-performance liquid chromatography (DHPLC). Methods of genotyping also include whole genome sequencing.

The term “sample” as used herein refers to a sample obtained from a patient, including a sample of biological tissue or fluid origin, obtained, reached, or collected in vivo or in situ. Exemplary samples include, but are not limited to, cell lysate, a cell culture, a cell line, a tissue, oral tissue, gastrointestinal tissue, an organ, an organelle, a biological fluid, a blood sample, a urine sample, a skin sample, and the like. In certain embodiments, biological samples include, but are not limited to, whole blood, partially purified blood, PBMCs, tissue biopsies, and the like.

The term “complex,” when used in reference to detection of a SNP, refers to a molecule in which one or more groups are linked by bonds, which includes a non-naturally occurring molecule, that is dependent upon the presence of the SNP in a sample. For example, a complex can include, but is not limited to, a hybridization complex, an enzyme-substrate complex, or an antibody-antigen complex. A “hybridization complex” refers to the interaction of two or more nucleic acid molecules by intermolecular forces including, but not limited to, covalent or non-covalent bonds, Van der Waals forces, dipole-dipole interactions, hydrogen bonding, and London dispersion forces.

The phrase “reaction product,” when used in reference to detection of a SNP, refers to a substance that is formed as a result of a chemical or biological reaction that is dependent upon the presence of the SNP in a sample. In some embodiments, the reaction product is a non-naturally occurring molecule. Such a non-naturally occurring molecule can be the results of methods well know in the art for detecting a SNP. Such methods include, but are not limited to, hybrization based methods (e.g., dynamic allele-specific hybrization, hybrization with a molecular beacon, or SNP microarrays), enzyme-based methods (e.g., restriction fragment length polymorphism (RFLP), polymerase chain reaction (PCR) based methods, flap endonuclease (FEN), primer extension methods, DNA polymerase with 5′-nuclease activity methods, or oligonucleotide ligation assays), physical property assays (e.g., single strand conformational polymorphism gel electrophoresis, temperature gradient gel electrophoresis, denaturing high performance liquid chromatography (DHPLC), high resolution melting analysis, or DNA mismatch-binding protein electrophoresis) and DNA sequencing (e.g., pyrosequencing, single-molecule real-time sequencing, ion torrent sequencing, sequencing by synthesis, sequencing by ligation, chemical degradation sequencing (Maxam-Gilbert sequencing), or chain termination sequencing (Sanger sequencing)).

By “solid support,” “solid substrate” or other grammatical equivalents herein refers to any material that contains and/or can be modified to contain one or more sites (e.g., discrete individual sites, pre-defined sites, random sites, etc.) appropriate for the attachment or association of compositions disclosed herein and is amenable to at least one detection method. As will be appreciated by those in the art, there are many possible substrates. Possible substrates include, but are not limited to, glass and modified or functionalized glass, plastics (including acrylics, polystyrene and copolymers of styrene and other materials, polypropylene, polyethylene, polybutylene, polyurethanes, Teflon®, etc.), polysaccharides, nylon or nitrocellulose, resins, silica or silica-based materials (including silicon and modified silicon), carbon, metals, inorganic glasses, plastics, optical fiber bundles, and a variety of other polymers. In general, the substrates can allow optical detection and do not themselves appreciably fluoresce.

### Peptides

Provided herein, in certain embodiments, are uses of chimeric and peptide sequences in the treatment and/or prevention of a gastrointestinal motility-related disorder. Provided herein, in certain embodiments, are uses of chimeric and peptide sequences in the treatment and/or prevention of constipation. Provided herein, in certain embodiments, are uses of chimeric and peptide sequences for stimulating bowel function. The invention is based, in part, on the use of variants of FGF19 peptide sequences, fusions of FGF19 and/or FGF21 peptide sequences and variants of fusions (chimeras) of FGF19 and/or FGF21 peptide sequences having one or more activities associated with the treatment and/or prevention of a gastrointestinal motility-related disorder. The invention is also based, in part, on the use of variants of FGF19 peptide sequences, fusions of FGF19 and/or FGF21 peptide sequences and variants of fusions (chimeras) of FGF19 and/or FGF21 peptide sequences having one or more activities associated with the treatment and/or prevention of constipation. Such variants and fusions (chimeras) of FGF19 and/or FGF21 peptide sequences include sequences that do not substantially increase or induce HCC formation or HCC tumorigenesis and/or do not induce a substantial elevation or increase in lipid profile.

In one embodiment, a chimeric peptide sequence comprises or consists of an N-terminal region having at least seven amino acid residues and the N-terminal region having a first amino acid position and a last amino acid position, where the N-terminal region has a DSSPL (SEQ ID NO:121) or DASPH (SEQ ID NO:122) sequence; and a C-terminal region having a portion of FGF19 and the C-terminal region having a first amino acid position and a last amino acid position, where the C-terminal region includes amino acid residues 16-29 of FGF19 (WGDPIRLRHLYTSG; SEQ ID NO:169) and the W residue corresponds to the first amino acid position of the C-terminal region. In particular embodiments, the variant is M70: MRDS SPLVHYGWGDPIRLRHLYTSGPHGLSSCFLRIRADGVVDCARGQSAHSLLEIKAVALR TVAIKGVHSVRYLCMGADGKMQGLLQYSEEDCAFEEEIRPDGYNVYRSEKHRLPVSLSSAK QRQLYKNRGFLPLSHFLPMLPMVPEEPEDLRGHLESDMFSSPLETDS16MDPFGLVTGLEAV RSPSFEK (SEQ ID NO:70). In other particular embodiments, the variant is M69: RDSSPLVHYGWGDPIRLRHLYTSGPHGLSSCFLRIRADGVVDCARGQSAHSLLEIKAVALRT VAIKGVHSVRYLCMGADGKMQGLLQYSEEDCAFEEEIRPDGYNVYRSEKHRLPVSLSSAKQ RQLYKNRGFLPLSHFLPMLPMVPEEPEDLRGHLESDMFSSPLETDS16MDPFGLVTGLEAVRS PSFEK (SEQ ID NO:69). In some embodiments, the N-terminal region has a DSSPL (SEQ ID NO:121). In other embodiments, the N-terminal region has a DASPH (SEQ ID NO:122) sequence.

In another embodiment, the treatment peptide, comprises: a) an N-terminal region comprising at least seven amino acid residues, the N-terminal region having a first amino acid position and a last amino acid position; and b) a C-terminal region comprising a portion of SEQ ID NO:99 [FGF19], the C-terminal region having a first amino acid position and a last amino acid position, wherein the C-terminal region comprises (i) a first C-terminal region sequence comprising WGDPIRLRHLYTSG (amino acids 16 to 29 of SEQ ID NO:99 [FGF19]), wherein the W residue corresponds to the first amino acid position of the C-terminal region; and (ii) a second C-terminal region sequence comprising PHGLSSCFLRIRADGVVDCARGQSAHSLLEIKAVALRTVAIKGVHSVRYLCMGADGKMQGL LQYSEEDCAFEEEIRPDGYNVYRSEKHRLPVSLSSAKQRQLYKNRGFLPLSHFLPMLPMVPE EPEDLRGHLESDMFSSPLETDSMDPFGLVTGLEAVRSPSFEK (amino acid residues 30 to 194 of SEQ ID NO:99 [FGF19]).

In another embodiment, the treatment peptide, comprises: a) an N-terminal region comprising at least seven amino acid residues, the N-terminal region having a first amino acid position and a last amino acid position, wherein the N-terminal region comprises DSSPL (SEQ ID NO:121) or DASPH (SEQ ID NO:122); and b) a C-terminal region comprising a portion of SEQ ID NO:99 [FGF19], the C-terminal region having a first amino acid position and a last amino acid position, wherein the C-terminal region comprises (i) a first C-terminal region sequence comprising WGDPIRLRHLYTSG (amino acids 16 to 29 of SEQ ID NO:99 [FGF19]), wherein the W residue corresponds to the first amino acid position of the C-terminal region; and (ii) a second C-terminal region sequence comprising PHGLSSCFLRIRADGVVDCARGQSAHSLLEIKAVALRTVAIKGVHSVRYLCMGADGKMQGL LQYSEEDCAFEEEIRPDGYNVYRSEKHRLPVSLSSAKQRQLYKNRGFLPLSHFLPMLPMVPE EPEDLRGHLESDMFSSPLETDSMDPFGLVTGLEAVRSPSFEK (amino acid residues 30 to 194 of SEQ ID NO:99 [FGF19]). In certain embodiments, the peptide (i) binds to FGFR4 with an affinity equal to or greater than FGF19 binding affinity for FGFR4; (ii) activates FGFR4 to an extent or amount equal to or greater than FGF19 activates FGFR4; (iii) has at least one of reduced HCC formation; greater glucose lowering activity, less lipid increasing activity, less triglyceride activity, less cholesterol activity, less non-HDL activity or less HDL increasing activity, as compared to FGF19, or as compared to a FGF19 variant sequence having any of GQV, GDI, WGPI (SEQ ID NO:171), WGDPV(SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the WGDPI (SEQ ID NO:170) sequence at amino acids 16-20 of FGF19 (SEQ ID NO:99); and/or (iv) has less lean mass reducing activity as compared to FGF21. In some embodiments, the N-terminal region has a DSSPL (SEQ ID NO:121). In other embodiments, the N-terminal region has a DASPH (SEQ ID NO:122) sequence.

In certain embodiments, the peptide decreases expression of the cytochrome P450 enzyme cholesterol 7α-hydroxylase (CYP7A1), which catalyzes the first rate-limiting step of hydroxylation of cholesterol to synthesize bile acids. In some embodiments, the reduction in bile acid synthesis by the peptide is demonstrated by a reduction in levels of serum 7-alpha-hydroxy-4-cholesten-3-one (C4, a downstream product of CYP7A1 action).

In certain embodiments, the second C-terminal region sequence comprises at least one amino acid substitution to the EIRPD (amino acids 2-6 of SEQ ID NO:190) sequence. In some embodiments, the at least one amino acid substitution is to the IRP sequence of the EIRPD (amino acids 2-6 of SEQ ID NO:190) sequence. In some embodiments, the at least one amino acid substitution is to the RP sequence of the EIRPD sequence (amino acids 2-6 of SEQ ID NO:190). In some embodiments, the at least one amino acid substitution is R to L substitution. In other embodiments, the at least one amino acid substitution is P to E substitution. In yet other embodiments, the at least one amino acid substitution is RP to LE substitution.

In some embodiments, the second C-terminal region sequence comprises from 2 to 5 amino acid substitutions, deletions or insertions. In other embodiments, the peptide is less than about 250 amino acids in length.

In one embodiment, the treatment peptide has an amino acid sequence comprising or consisting of MRDSSPLVHYGWGDPIRLRHLYTSGPHGLSSCFLRIRADGVVDCARGQSAHSLLEIKAVALR TVAIKGVHSVRYLCMGADGKMQGLLQYSEEDCAFEEEIRPDGYNVYRSEKHRLPVSLSSAK QRQLYKNRGFLPLSHFLPMLPMVPEEPEDLRGHLESDMFSSPLETDSMDPFGLVTGLEAVRS PSFEK (SEQ ID NO:70). In certain embodiments, the treatment peptide has an amino acid sequence comprising SEQ ID NO:70. In other embodiments, the treatment peptide has an amino acid sequence consisting of SEQ ID NO:70. In some embodiments, the treatment peptide is fused with an immunoglobulin Fc region.

In another embodiment, the treatment peptide has an amino acid sequence comprising or consisting of RDSSPLVHYGWGDPIRLRHLYTSGPHGLSSCFLRIRADGVVDCARGOSAHSLLEIKAVALRT VAIKGVHSVRYLCMGADGKMQGLLQYSEEDCAFEEEIRPDGYNVYRSEKHRLPVSLSSAKQ RQLYKNRGFLPLSHFLPMLPMVPEEPEDLRGHLESDMFSSPLETDSMDPFGLVTGLEAVRSP SFEK (SEQ ID NO:69). In certain embodiments, the treatment peptide has an amino acid sequence comprising SEQ ID NO:69. In other embodiments, the treatment peptide has an amino acid sequence consisting of SEQ ID NO:69. In some embodiments, the treatment peptide is fused with an immunoglobulin Fc region.

In another embodiment, the treatment peptide, comprises: a) an N-terminal region comprising at least seven amino acid residues, the N-terminal region having a first amino acid position and a last amino acid position; and b) a C-terminal region comprising a first amino acid position and a last amino acid position, wherein the C-terminal region comprises (i) a first C-terminal region sequence comprising WGDPIRQRHLYTSG (SEQ ID NO:169 with a L7Q substitution), wherein the W residue corresponds to the first amino acid position of the C-terminal region; and (ii) a second C-terminal region sequence comprising

(SEQ ID NO: 188)
PHGLSSCFLRIRADGVVDCARGQSAHSLLEIKAVALRTVAIKGVHSVRYL
CMGADGKMQGLLQYSEEDCAFEEEIRPDGYNVYRSEKHRLPVSLSSAKQR
QLYKNRGFLPLSHFLPMLPMVPEEPEDLRGHLESDMFSSPLETDSMDPFG
LVTGLEAVRSPSFEK.

In another embodiment, the treatment peptide, comprises: a) an N-terminal region comprising at least seven amino acid residues, the N-terminal region having a first amino acid position and a last amino acid position, wherein the N-terminal region comprises DSSPL (SEQ ID NO:121), DASPH (SEQ ID NO:122), or DAGPH (amino acids 7 to 11 of SEQ ID NO:99 [FGF19]); and b) a C-terminal region comprising a first amino acid position and a last amino acid position, wherein the C-terminal region comprises (i) a first C-terminal region sequence comprising WGDPIRQRHLYTSG (SEQ ID NO:169 with a L7Q substitution), wherein the W residue corresponds to the first amino acid position of the C-terminal region; and (ii) a second C-terminal region sequence comprising PHGLSSCFLRIRADGVVDCARGQSAHSLLEIKAVALRTVAIKGVHSVRYLCMGADGKMQGL LQYSEEDCAFEEEIRPDGYNVYRSEKHRLPVSLSSAKQRQLYKNRGFLPLSHFLPMLPMVPE EPEDLRGHLESDMFSSPLETDSMDPFGLVTGLEAVRSPSFEK (SEQ ID NO:188). In some embodiments, the peptide (i) binds to FGFR4 with an affinity equal to or greater than FGF19 binding affinity for FGFR4; (ii) activates FGFR4 to an extent or amount equal to or greater than FGF19 activates FGFR4; (iii) has at least one of reduced hepatocellular carcinoma (HCC) formation; greater glucose lowering activity, less lipid increasing activity, less triglyceride activity, less cholesterol activity, less non-HDL activity or less HDL increasing activity, as compared to FGF19, or as compared to a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI(SEQ ID NO:182), WGDP (SEQ ID NO:183) or FGDPI (SEQ ID NO:184) substituted for the FGF19 WGDPI (SEQ ID NO:170) sequence at amino acids 16-20; and/or (iv) has less lean mass reducing activity as compared to FGF21.

In certain embodiments, the second C-terminal region sequence comprises at least one amino acid substitution to the EIRPD (amino acids 2-6 of SEQ ID NO:190) sequence. In some embodiments, the at least one amino acid substitution is to the IRP sequence of the EIRPD (amino acids 2-6 of SEQ ID NO:190) sequence. In some embodiments, the at least one amino acid substitution is to the RP sequence of the EIRPD sequence (amino acids 2-6 of SEQ ID NO:190). In some embodiments, the at least one amino acid substitution is R to L substitution. In other embodiments, the at least one amino acid substitution is P to E substitution. In yet other embodiments, the at least one amino acid substitution is RP to LE substitution.

In some embodiments, the second C-terminal region sequence comprises from 2 to 5 amino acid substitutions, deletions or insertions. In other embodiments, the peptide is less than about 250 amino acids in length.

In another embodiment, a chimeric peptide sequence comprises or consists of an N-terminal region having a portion of FGF21 and the N-terminal region having a first amino acid position and a last amino acid position, where the N-terminal region has a GQV sequence and the V residue corresponds to the last amino acid position of the N-terminal region; and a C-terminal region having a portion of FGF19 and the C-terminal region having a first amino acid position and a last amino acid position where the C-terminal region includes amino acid residues 21-29 of FGF19 (RLRHLYTSG; SEQ ID NO: 185) and the R residue corresponds to the first position of the C-terminal region.

In particular aspects, modifications to the Loop-8 region of FGF19 are disclosed herein that possess favorable metabolic parameters without exhibiting substantial tumorigenicity. Herein, FGF19 residues 127-129 are defined as constituting the Loop-8 region, although in the literature the Loop-8 region is sometimes defined as including or consisting of other residues (e.g., residues 125-129). Certain combinations of R127L and P128E substitutions to the FGF19 framework had an unexpectedly positive effect on HCC formation. Even more surprisingly, a combination of R127L and P128E substitutions and a substitution of Gln (Q) for Leu (L) in the FGF19 core region had an even more significant effect on preventing HCC formation. Accordingly, variants of FGF19 Loop-8 region are included since they can reduce or eliminate substantial, measurable or detectable HCC formation. Furthermore, the effect of reducing HCC formation may be enhanced by modifications to amino acid residues outside of the Loop 8 region (e.g., substitutions of amino acid residues in the core region).

Accordingly, variants of FGF19 Loop-8 region are included since they can reduce or eliminate substantial, measurable or detectable HCC formation. Furthermore, the effect of reducing HCC formation may be enhanced by modifications to amino acid residues outside of the Loop-8 region (e.g., substitutions of amino acid residues in the core region, such as the region corresponding to amino acids 21-29 of SEQ ID NO:99). In some embodiments, the Loop-8 modified variant comprises a substitution in the FGF19 Loop-8 region corresponding to amino acids 127-129 of SEQ ID NO:99. In certain embodiments, the Loop-8 modified variant comprises a substitution in the FGF19 Loop-8 region corresponding to (i) a R127L substitution, (ii) a P128E substitution, or (iii) a R127L substitution and a P128E substitution.

In certain embodiments, the amino acid sequence of the peptide comprises at least one amino acid substitution in the Loop-8 region of FGF19, or the corresponding FGF19 sequence thereof in a variant peptide provided herein. In certain embodiments, the amino acid sequence of the peptide comprises one amino acid substitution to the EIRPD (amino acids 2-6 of SEQ ID NO:190) amino acid sequence in the Loop-8 region of FGF19. In some embodiments, the amino acid sequence of the peptide comprises two amino acid substitutions to the EIRPD (amino acids 2-6 of SEQ ID NO:190) amino acid sequence in the Loop-8 region of FGF19. In other embodiments, the amino acid sequence of the peptide comprises three amino acid substitutions to the EIRPD (amino acids 2-6 of SEQ ID NO:190) amino acid sequence in the Loop-8 region of FGF19. In certain embodiments, the amino acid sequence of the peptide comprises four amino acid substitutions to the EIRPD (amino acids 2-6 of SEQ ID NO:190) amino acid sequence in the Loop-8 region of FGF19. In some embodiments, the amino acid sequence of the peptide comprises five amino acid substitutions to the EIRPD (amino acids 2-6 of SEQ ID NO:190) amino acid sequence in the Loop-8 region of FGF19. In certain embodiments, the amino acid sequence of the peptide comprises one amino acid substitution to the IRP (amino acids 3-5 of SEQ ID NO:190) amino acid sequence in the Loop-8 region of FGF19. In some embodiments, the amino acid sequence of the peptide comprises two amino acid substitutions to the IRP (amino acids 3-5 of SEQ ID NO:190) amino acid sequence in the Loop-8 region of FGF19. In other embodiments, the amino acid sequence of the peptide comprises three amino acid substitutions to the IRP (amino acids 3-5 of SEQ ID NO:190) amino acid sequence in the Loop-8 region of FGF19. In certain embodiments, the amino acid sequence of the peptide comprises one amino acid substitution to the RP (amino acids 4-5 of SEQ ID NO:190) amino acid sequence in the Loop-8 region of FGF19. In some embodiments, the amino acid sequence of the peptide comprises two amino acid substitutions to the RP (amino acids 4-5 of SEQ ID NO:190) amino acid sequence in the Loop-8 region of FGF19. In certain embodiments, the amino acid substitution to the RP (amino acids 4-5 of SEQ ID NO:190) amino acid sequence in the Loop-8 region of FGF19 is an Arg (R) to Leu (L) substitution. In other embodiments, the substitution to the RP (amino acids 4-5 of SEQ ID NO:190) amino acid sequence in the Loop-8 region of FGF19 is a Pro (P) to Glu (E) substitution. In some embodiments, the substitutions to the RP (amino acids 4-5 of SEQ ID NO:190) amino acid sequence in the Loop-8 region of FGF19 is an Arg (R) to Leu (L) substitution and a Pro (P) to Glu (E) substitution. In specific embodiments, the foregoing substitution(s) in the Loop-8 region of FGF19 is in the corresponding FGF19 sequence thereof in a variant peptide provided herein. That is, said substitutions within a corresponding FGF19 sequence (e.g., EIRPD, IRP or RP) of a peptide variant provided herein is also contemplated.

In some embodiments, the FGF19 variant comprises or further comprises a substitution in the core region corresponding to amino acids 21-29 of SEQ ID NO:99. In certain embodiments, the FGF19 variant comprises or further comprises a substitution in the core region corresponding to a L22Q substitution.

In some embodiments, the Loop-8 modified variant is M70: MRDSSPLVHYGWGDPIRLRHLYTSGPHGLSSCFLRIRADGVVDCARGQSAHSLLEIKAVALR TVAIKGVHSVRYLCMGADGKMQGLLQYSEEDCAFEEEIRPDGYNVYRSEKHRLPVSLSSAK QRQLYKNRGFLPLSHFLPMLPMVPEEPEDLRGHLESDMFSSPLETDS16MDPFGLVTGLEAV RSPSFEK (SEQ ID NO:70), comprising a substitution in the FGF19 Loop-8 region (underlined). In certain embodiments, the Loop-8 modified M70 variant comprises a substitution in the FGF19 Loop-8 region (RPD; underlined) corresponding to (i) an R to L substitution, (ii) a P to E substitution, or (iii) an R to L substitution and a P to E substitution (SEQ. ID NO:204). In certain embodiments, the Loop-8 modified M70 variant further comprises or further comprises a substitution in the FGF19 core region. In some embodiments, the Loop-8 modified M70 variant comprises a L18Q substitution (i.e., SEQ ID NO:70 with an L18Q substitution).

In some embodiments, the Loop-8 modified variant is M69: RDSSPLVHYGWGDPIRLRHLYTSGPHGLSSCFLRIRADGVVDCARGQSAHSLLEIKAVALRT VAIKGVHSVRYLCMGADGKMQGLLQYSEEDCAFEEEIRPDGYNVYRSEKHRLPVSLSSAKQ RQLYKNRGFLPLSHFLPMLPMVPEEPEDLRGHLESDMFSSPLETDS16MDPFGLVTGLEAVRS PSFEK (SEQ ID NO:69), comprising a substitution in the FGF19 Loop-8 region (underlined). In certain embodiments, the Loop-8 modified M69 variant comprises a substitution in the FGF19 Loop-8 region (RPD; underlined) corresponding to (i) an R to L substitution, (ii) a P to E substitution, or (iii) an R to L substitution and a P to E substitution. In certain embodiments, the Loop-8 modified M69 variant further comprises or further comprises a substitution in the FGF19 core region. In some embodiments, the Loop-8 modified M69 variant comprises a L17Q substitution (i.e., SEQ ID NO:69 with an L17Q substitution).

Other counterpart modifications in other variants provided herein are also contemplated. In certain embodiments, the amino acid sequence of the peptide comprises one amino acid substitution to the EIRPD (amino acids 2-6 of SEQ ID NO:190) amino acid sequence in the Loop-8 region of FGF19. In some embodiments, the amino acid sequence of the peptide comprises two amino acid substitutions to the EIRPD (amino acids 2-6 of SEQ ID NO:190) amino acid sequence in the Loop-8 region of FGF19. In other embodiments, the amino acid sequence of the peptide comprises three amino acid substitutions to the EIRPD (amino acids 2-6 of SEQ ID NO:190) amino acid sequence in the Loop-8 region of FGF19. In certain embodiments, the amino acid sequence of the peptide comprises four amino acid substitutions to the EIRPD (amino acids 2-6 of SEQ ID NO:190) amino acid sequence in the Loop-8 region of FGF19. In some embodiments, the amino acid sequence of the peptide comprises five amino acid substitutions to the EIRPD (amino acids 2-6 of SEQ ID NO:190) amino acid sequence in the Loop-8 region of FGF19. In certain embodiments, the amino acid sequence of the peptide comprises one amino acid substitution to the IRP (amino acids 3-5 of SEQ ID NO:190) amino acid sequence in the Loop-8 region of FGF19. In some embodiments, the amino acid sequence of the peptide comprises two amino acid substitutions to the IRP (amino acids 3-5 of SEQ ID NO:190) amino acid sequence in the Loop-8 region of FGF19. In other embodiments, the amino acid sequence of the peptide comprises three amino acid substitutions to the IRP (amino acids 3-5 of SEQ ID NO:190) amino acid sequence in the Loop-8 region of FGF19. In certain embodiments, the amino acid sequence of the peptide comprises one amino acid substitution to the RP (amino acids 4-5 of SEQ ID NO:190) amino acid sequence in the Loop-8 region of FGF19. In some embodiments, the amino acid sequence of the peptide comprises two amino acid substitutions to the RP (amino acids 4-5 of SEQ ID NO:190) amino acid sequence in the Loop-8 region of FGF19. In certain embodiments, the amino acid substitution to the RP (amino acids 4-5 of SEQ ID NO:190) amino acid sequence in the Loop-8 region of FGF19 is an Arg (R) to Leu (L) substitution. In other embodiments, the substitution to the RP (amino acids 4-5 of SEQ ID NO:190) amino acid sequence in the Loop-8 region of FGF19 is a Pro (P) to Glu (E) substitution. In some embodiments, the substitutions to the RP (amino acids 4-5 of SEQ ID NO:190) amino acid sequence in the Loop-8 region of FGF19 is an Arg (R) to Leu (L) substitution and a Pro (P) to Glu (E) substitution. In specific embodiments, the foregoing substitution(s) in the Loop-8 region of FGF19 is in the corresponding FGF19 sequence thereof in a variant peptide provided herein. That is, said substitutions within a corresponding FGF19 sequence (e.g., EIRPD, IRP or RP) of a peptide variant provided herein is also contemplated.

In further embodiments, a peptide sequence includes or consists of a FGF19 variant having one or more amino acid substitutions, insertions or deletions compared to a reference or wild type FGF19. In additional embodiments, a peptide sequence includes or consists of a FGF21 sequence variant having one or more amino acid substitutions, insertions or deletions compared to a reference or wild type FGF21. In yet additional embodiments, a peptide sequence includes or consists of a portion of a FGF19 sequence fused to a portion of a FGF21 sequence. In still additional embodiments, a peptide sequence includes or consists of a portion of a FGF19 sequence fused to a portion of a FGF21 sequence, where the FGF19 and/or FGF21 sequence portion(s) have one or more amino acid substitutions, insertions or deletions compared to a reference or wild type FGF19 and/or FGF21. Examples of such sequences are disclosed in PCT Pub. No. WO 2013/006486 and US Pub. No. 2013/0023474, as well as PCT Publ. No. WO 2014/085365, published Jun. 5, 2014. Table 1 and the Sequence Listing also sets forth representative sequences that may be used in the methods provided herein.

In some embodiments, the treatment peptides provided herein include variants and fusions of FGF19 and/or FGF21 peptide sequences. In one embodiment, the treatment peptides include one or more variant or fusion FGF19 and/or FGF21 peptide. In other embodiments, the methods provided herein include contacting or administering to a subject one or more nucleic acid molecules encoding a variant or fusion FGF19 and/or FGF21 peptide sequence (for example, an expression control element in operable linkage with the nucleic acid encoding the peptide sequence, optionally including a vector), in an amount effective for treating a bile acid-related or associated disorder.

A representative reference or wild type FGF19 sequence is set forth as:

(SEQ ID NO: 99)
RPLAFSDAGPHVHYGWGDPIRLRHLYTSGPHGLSSCFLRIRADGVVDCAR
GQSAHSLLEIKAVALRTVAIKGVHSVRYLCMGADGKMQGLLQYSEEDCAF
EEEIRPDGYNVYRSEKEIRLPVSLSSAKQRQLYKNRGFLPLSHFLPMLPM
VPEEPEDLRGHLESDMFSSPLETDSMDPFGLVTGLEAVRSPSFEK.

A representative reference or wild type FGF21 sequence is set forth as: HPIPDSSPLLQFGGQVRQRYLYTDDAQQTEAHLEIREDGTVGGAADQSPESLLQLKALKPGV IQILGVKTSRFLCQRPDGALYGSLHFDPEACSFRELLLEDGYNVYQSEAHGLPLHLPGNKSPH RDPAPRGPARFLPLPGLPPALPEPPGILAPQPPDVGSSDPLSMVGPSQGRSPSYAS (SEQ ID NO:100). FGF21 allelic variants include, e.g., M70, M71 and M72.

In further embodiments, a peptide sequence comprises or consists of a FGF19 variant having one or more amino acid substitutions, insertions or deletions compared to a reference or wild type FGF19. In additional embodiments, a peptide sequence comprises or consists of a FGF21 sequence variant having one or more amino acid substitutions, insertions or deletions compared to a reference or wild type FGF21. In yet additional embodiments, a peptide sequence comprises or consists of a portion of a FGF19 sequence fused to a portion of a FGF21 sequence. In still additional embodiments, a peptide sequence comprises or consists of a portion of a FGF19 sequence fused to a portion of a FGF21 sequence, where the FGF19 and/or FGF21 sequence portion(s) have one or more amino acid substitutions, insertions or deletions compared to a reference or wild type FGF19 and/or FGF21. Examples of such sequences are disclosed in PCT Pub. No. WO 2013/006486 and US Pub. No. 2013/0023474, as well as PCT Publ. No. WO 2014/085365, published Jun. 5, 2014. Table 1 and the Sequence Listing also sets forth representative sequences that may be used in the methods provided herein.

In various particular aspects, a peptide or chimeric sequence provided herein has at the N-terminal region first amino acid position an “M” residue, an “R” residue, a “S” residue, a “H” residue, a “P” residue, a “L” residue or an “D” residue. In various alternative particular aspects, a peptide or chimeric sequence peptide sequence does not have a “M” residue or an “R” residue at the first amino acid position of the N-terminal region.

Typically, the number of amino acids or residues in a peptide sequence provided herein will total less than about 250 (e.g., amino acids or mimetics thereof). In various particular embodiments, the number of residues comprise from about 20 up to about 200 residues (e.g., amino acids or mimetics thereof). In additional embodiments, the number of residues comprise from about 50 up to about 200 residues (e.g., amino acids or mimetics thereof). In further embodiments, the number of residues comprise from about 100 up to about 195 residues (e.g., amino acids or mimetics thereof) in length.

Amino acids or residues can be linked by amide or by non-natural and non-amide chemical bonds including, for example, those formed with glutaraldehyde, N-hydroxysuccinimide esters, bifunctional maleimides, or N, N′-dicyclohexylcarbodiimide (DCC). Non-amide bonds include, for example, ketomethylene, aminomethylene, olefin, ether, thioether and the like (see, e.g., Spatola in Chemistry and Biochemistry of Amino Acids, Peptides and Proteins, Vol. 7, pp 267-357 (1983), “Peptide and Backbone Modifications,” Marcel Decker, NY). Thus, when a peptide provided herein includes a portion of a FGF19 sequence and a portion of a FGF21 sequence, the two portions need not be joined to each other by an amide bond, but can be joined by any other chemical moiety or conjugated together via a linker moiety.

Also provided herein are subsequences, variants and modified forms of the exemplified peptide sequences (including the FGF19 and FGF21 variants and subsequences listed in the Sequence Listing, or Table 1), so long as the foregoing retains at least a detectable or measureable activity or function. Also, certain exemplified variant peptides, for example, those having all or a portion of FGF21 sequence at the amino-terminus, have an “R” residue positioned at the N-terminus, which can be omitted. Similarly, certain exemplified variant peptides, include an “M” residue positioned at the N-terminus, which can be appended to or further substituted for an omitted residue, such as an “R” residue. More particularly, in various embodiments peptide sequences at the N-terminus include any of: RDSS (SEQ ID NO:115), DSS, MDSS (SEQ ID NO:116) or MRDSS (SEQ ID NO:117). Furthermore, when a “M” residue is adjacent to a “S” residue, the “M” residue may be cleaved such that the “M” residue is deleted from the peptide sequence, whereas when the “M” residue is adjacent to a “D” residue, the “M” residue may not be cleaved. Thus, by way of example, in various embodiments peptide sequences include those with the following residues at the N-terminus: MDSSPL (SEQ ID NO:119), MSDSSPL (SEQ ID NO:120) (cleaved to SDSSPL (SEQ ID NO:112)) and MSSPL (SEQ ID NO:113) (cleaved to SSPL (SEQ ID NO:114)).

As used herein, the term “modify” and grammatical variations thereof, means that the composition deviates relative to a reference composition, such as a peptide sequence. Such modified peptide sequences, nucleic acids and other compositions may have greater or less activity or function, or have a distinct function or activity compared with a reference unmodified peptide sequence, nucleic acid, or other composition, or may have a property desirable in a protein formulated for therapy (e.g. serum half-life), to elicit antibody for use in a detection assay, and/or for protein purification. For example, a peptide sequence provided herein can be modified to increase serum half-life, to increase in vitro and/or in vivo stability of the protein, etc.

Particular examples of such subsequences, variants and modified forms of the peptide sequences exemplified herein (e.g., a peptide sequence listed in the Sequence Listing or Table 1) include substitutions, deletions and/or insertions/additions of one or more amino acids, to or from the amino-terminus, the carboxy-terminus or internally. One example is a substitution of an amino acid residue for another amino acid residue within the peptide sequence. Another is a deletion of one or more amino acid residues from the peptide sequence, or an insertion or addition of one or more amino acid residues into the peptide sequence.

The number of residues substituted, deleted or inserted/added are one or more amino acids (e.g., 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250, or more) of a peptide sequence. Thus, a FGF19 or FGF21 sequence can have few or many amino acids substituted, deleted or inserted/added (e.g., 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250, or more). In addition, a FGF19 amino acid sequence can include or consist of an amino acid sequence of about 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250, or more amino acids from FGF21; or a FGF21 amino acid or sequence can include or consist of an amino acid sequence of about 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250, or more amino acids from FGF19.

Specific examples of substitutions include substituting a D residue for an L-residue. Accordingly, although residues are listed in the L-isomer configuration, D-amino acids at any particular or all positions of the peptide sequences provided herein are included, unless a D-isomer leads to a sequence that has no detectable or measurable function.

Additional specific examples are non-conservative and conservative substitutions. A “conservative substitution” is a replacement of one amino acid by a biologically, chemically or structurally similar residue. Biologically similar means that the substitution is compatible with a biological activity, e.g., activity that ameliorate a gastrointestinal motility-related disorder and/or the manifestations thereof. Structurally similar means that the amino acids have side chains with similar length, such as alanine, glycine and serine, or having similar size, or the structure of a first, second or additional peptide sequence is maintained. Chemical similarity means that the residues have the same charge or are both hydrophilic and hydrophobic. Particular examples include the substitution of one hydrophobic residue, such as isoleucine, valine, leucine or methionine, for another, or the substitution of one polar residue for another, such as the substitution of arginine for lysine, glutamic for aspartic acids, or glutamine for asparagine, serine for threonine, etc. Routine assays can be used to determine whether a subsequence, variant or modified form has activity, e.g., activity that ameliorate a gastrointestinal motility-related disorder and/or the manifestations thereof.

Particular examples of subsequences, variants and modified forms of the peptide sequences exemplified herein have 50%-60%, 60%-70%, 70%-75%, 75%-80%, 80%-85%, 85%-90%, 90%-95%, or 96%, 97%, 98%, or 99% identity to a reference peptide sequence. The term “identity” and “homology” and grammatical variations thereof mean that two or more referenced entities are the same. Thus, where two amino acid sequences are identical, they have the identical amino acid sequence. “Areas, regions or domains of identity” mean that a portion of two or more referenced entities are the same. Thus, where two amino acid sequences are identical or homologous over one or more sequence regions, they share identity in those regions.

The extent of identity between two sequences can be ascertained using a computer program and mathematical algorithm known in the art. Such algorithms that calculate percent sequence identity (homology) generally account for sequence gaps and mismatches over the comparison region. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For peptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

In the peptide sequences, including subsequences, variants and modified forms of the peptide sequences exemplified herein, an “amino acid” or “residue” includes conventional alpha-amino acids as well as beta-amino acids; alpha, alpha disubstituted amino acids; and N-substituted amino acids, wherein at least one side chain is an amino acid side chain moiety as defined herein. An “amino acid” further includes N-alkyl alpha-amino acids, wherein the N-terminus amino group has a C₁ to C₆ linear or branched alkyl substituent. The term “amino acid” therefore includes stereoisomers and modifications of naturally occurring protein amino acids, non-protein amino acids, post-translationally modified amino acids (e.g., by glycosylation, phosphorylation, ester or amide cleavage, etc.), enzymatically modified or synthesized amino acids, derivatized amino acids, constructs or structures designed to mimic amino acids, amino acids with a side chain moiety modified, derivatized from naturally occurring moieties, or synthetic, or not naturally occurring, etc. Modified and unusual amino acids are included in the peptide sequences provided herein (see, for example, in Synthetic Peptides: A User's Guide; Hruby et al., Biochem. J. 268:249 (1990); and Toniolo C., Int. J. Peptide Protein Res. 35:287 (1990)).

In addition, protecting and modifying groups of amino acids are included. The term “amino acid side chain moiety” as used herein includes any side chain of any amino acid, as the term “amino acid” is defined herein. This therefore includes the side chain moiety in naturally occurring amino acids. It further includes side chain moieties in modified naturally occurring amino acids as set forth herein and known to one of skill in the art, such as side chain moieties in stereoisomers and modifications of naturally occurring protein amino acids, non-protein amino acids, post-translationally modified amino acids, enzymatically modified or synthesized amino acids, derivatized amino acids, constructs or structures designed to mimic amino acids, etc. For example, the side chain moiety of any amino acid disclosed herein or known to one of skill in the art is included within the definition.

A “derivative of an amino acid side chain moiety” is included within the definition of an amino acid side chain moiety. Non-limiting examples of derivatized amino acid side chain moieties include, for example: (a) adding one or more saturated or unsaturated carbon atoms to an existing alkyl, aryl, or aralkyl chain; (b) substituting a carbon in the side chain with another atom, such as oxygen or nitrogen; (c) adding a terminal group to a carbon atom of the side chain, including methyl (—CH₃), methoxy (—OCH₃), nitro (—NO₂), hydroxyl (—OH), or cyano (—C═N); (d) for side chain moieties including a hydroxy, thiol or amino groups, adding a suitable hydroxy, thiol or amino protecting group; or (e) for side chain moieties including a ring structure, adding one or more ring substituents, including hydroxyl, halogen, alkyl, or aryl groups attached directly or through, e.g., an ether linkage. For amino groups, suitable protecting groups are known to the skilled artisan. Provided such derivatization provides a desired activity in the final peptide sequence (e.g., activity that ameliorate a gastrointestinal motility-related disorder and/or the manifestations thereof).

An “amino acid side chain moiety” includes all such derivatization, and particular non-limiting examples include: gamma-amino butyric acid, 12-amino dodecanoic acid, alpha-aminoisobutyric acid, 6-amino hexanoic acid, 4-(aminomethyl)-cyclohexane carboxylic acid, 8-amino octanoic acid, biphenylalanine, Boc—t-butoxycarbonyl, benzyl, benzoyl, citrulline, diaminobutyric acid, pyrrollysine, diaminopropionic acid, 3,3-diphenylalanine, orthonine, citrulline, 1,3-dihydro-2H-isoindolecarboxylic acid, ethyl, Fmoc—fluorenylmethoxycarbonyl, heptanoyl (CH₃—(CH₂)₅—C(═O)—), hexanoyl (CH₃—(CH₂)₄—C(═O)—), homoarginine, homocysteine, homolysine, homophenylalanine, homoserine, methyl, methionine sulfoxide, methionine sulfone, norvaline (NVA), phenylglycine, propyl, isopropyl, sarcosine (SAR), tert-butylalanine, and benzyloxycarbonyl.

A single amino acid, including stereoisomers and modifications of naturally occurring protein amino acids, non-protein amino acids, post-translationally modified amino acids, enzymatically-synthesized amino acids, non-naturally occurring amino acids including derivatized amino acids, an alpha, alpha disubstituted amino acid derived from any of the foregoing (i.e., an alpha, alpha disubstituted amino acid, wherein at least one side chain is the same as that of the residue from which it is derived), a beta-amino acid derived from any of the foregoing (i.e., a beta-amino acid which, other than for the presence of a beta-carbon, is the same as the residue from which it is derived) etc., including all of the foregoing can be referred to herein as a “residue.” Suitable substituents, in addition to the side chain moiety of the alpha-amino acid, include C₁ to C₆ linear or branched alkyl. Aib is an example of an alpha, alpha disubstituted amino acid. While alpha, alpha disubstituted amino acids can be referred to using conventional L- and D-isomeric references, it is to be understood that such references are for convenience, and that where the substituents at the alpha-position are different, such amino acid can interchangeably be referred to as an alpha, alpha disubstituted amino acid derived from the L- or D-isomer, as appropriate, of a residue with the designated amino acid side chain moiety. Thus (S)-2-Amino-2-methyl-hexanoic acid can be referred to as either an alpha, alpha disubstituted amino acid derived from L-Nle (norleucine) or as an alpha, alpha disubstituted amino acid derived from D-Ala. Similarly, Aib can be referred to as an alpha, alpha disubstituted amino acid derived from Ala. Whenever an alpha, alpha disubstituted amino acid is provided, it is to be understood as including all (R) and (S) configurations thereof.

An “N-substituted amino acid” includes any amino acid wherein an amino acid side chain moiety is covalently bonded to the backbone amino group, optionally where there are no substituents other than H in the alpha-carbon position. Sarcosine is an example of an N-substituted amino acid. By way of example, sarcosine can be referred to as an N-substituted amino acid derivative of Ala, in that the amino acid side chain moiety of sarcosine and Ala is the same, i.e., methyl.

In certain embodiments, covalent modifications of the peptide sequences, including subsequences, variants and modified forms of the peptide sequences exemplified herein are provided. An exemplary type of covalent modification includes reacting targeted amino acid residues with an organic derivatizing agent that is capable of reacting with selected side chains or the N- or C-terminal residues of the peptide. Derivatization with bifunctional agents is useful, for instance, for cross-linking peptide to a water-insoluble support matrix or surface for use in the method for purifying anti-peptide antibodies, and vice-versa. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, amidation of any C-terminal carboxyl group, etc.

Exemplified peptide sequences, and subsequences, variants and modified forms of the peptide sequences exemplified herein can also include alterations of the backbone for stability, derivatives, and peptidomimetics. The term “peptidomimetic” includes a molecule that is a mimic of a residue (referred to as a “mimetic”), including but not limited to piperazine core molecules, keto-piperazine core molecules and diazepine core molecules. Unless otherwise specified, an amino acid mimetic of a peptide sequence provided herein includes both a carboxyl group and amino group, and a group corresponding to an amino acid side chain, or in the case of a mimetic of Glycine, no side chain other than hydrogen.

By way of example, these would include compounds that mimic the sterics, surface charge distribution, polarity, etc. of a naturally occurring amino acid, but need not be an amino acid, which would impart stability in the biological system. For example, Proline may be substituted by other lactams or lactones of suitable size and substitution; Leucine may be substituted by an alkyl ketone, N-substituted amide, as well as variations in amino acid side chain length using alkyl, alkenyl or other substituents, others may be apparent to the skilled artisan. The essential element of making such substitutions is to provide a molecule of roughly the same size and charge and configuration as the residue used to design the molecule. Refinement of these modifications will be made by analyzing the compounds in a functional (e.g., glucose lowering) or other assay, and comparing the structure-activity relationship. Such methods are within the scope of the skilled artisan working in medicinal chemistry and drug development.

The term “bind,” or “binding,” when used in reference to a peptide sequence, means that the peptide sequence interacts at the molecular level. Specific and selective binding can be distinguished from non-specific binding using assays known in the art (e.g., competition binding, immunoprecipitation, ELISA, flow cytometry, Western blotting).

Peptides and peptidomimetics can be produced and isolated using methods known in the art. Peptides can be synthesized, in whole or in part, using chemical methods (see, e.g., Caruthers (1980). Nucleic Acids Res. Symp. Ser. 215; Horn (1980); and Banga, A. K., Therapeutic Peptides and Proteins, Formulation, Processing and Delivery Systems (1995) Technomic Publishing Co., Lancaster, Pa.). Peptide synthesis can be performed using various solid-phase techniques (see, e.g., Roberge Science 269:202 (1995); Merrifield, Methods Enzymol. 289:3 (1997)) and automated synthesis may be achieved, e.g., using the ABI 431A Peptide Synthesizer (Perkin Elmer) in accordance with the manufacturer's instructions. Peptides and peptide mimetics can also be synthesized using combinatorial methodologies. Synthetic residues and polypeptides incorporating mimetics can be synthesized using a variety of procedures and methodologies known in the art (see, e.g., Organic Syntheses Collective Volumes, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR-based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al, Gene 34:315 (1985)), restriction selection mutagenesis (Wells et al, Philos. Trans. R. Soc. London SerA 317:415 (1986)) and other techniques can be performed on cloned DNA to produce peptide sequences, variants, fusions and chimeras provided herein, and variations, derivatives, substitutions and modifications thereof.

A “synthesized” or “manufactured” peptide sequence is a peptide made by any method involving manipulation by the hand of man. Such methods include, but are not limited to, the aforementioned, such as chemical synthesis, recombinant DNA technology, biochemical or enzymatic fragmentation of larger molecules, and combinations of the foregoing.

Peptide sequences provided herein including subsequences, sequence variants and modified forms of the exemplified peptide sequences (e.g., sequences listed in the Sequence Listing or Table 1), can also be modified to form a chimeric molecule. In certain embodiments, provided herein are peptide sequences that include a heterologous domain. Such domains can be added to the amino-terminus or at the carboxyl-terminus of the peptide sequence. Heterologous domains can also be positioned within the peptide sequence, and/or alternatively flanked by FGF19 and/or FGF21 derived amino acid sequences.

The term “peptide” also includes dimers or multimers (oligomers) of peptides. In certain embodiments, dimers or multimers (oligomers) of the exemplified peptide sequences are provided herein, as well as subsequences, variants and modified forms of the exemplified peptide sequences, including sequences listed in the Sequence Listing or Table 1.

In certain embodiments, a peptide sequence provided herein comprises an amino acid sequence set forth in Table 1. In other embodiments, a peptide sequence provided herein consists of an amino acid sequence set forth in Table 1.

TABLE 1

| SEQ | |
|---|---|
| ID | |
| NO. | Amino Acid Sequence |
| 1. | RPLAFSDASPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 2. | RPLAFSDSSPLVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 3. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEILEDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 4. | RPLAFSDAGPHVHYAWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 5. | RHPIPDSSPLLQFGGQVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 6. | RDSSPLLQFGGQVRLRHLYTSGPHGLSSCFLRIRADGVVDCARGQSAHSLL |
| | EIKAVALRTVAIKGVHSVRYLCMGADGKMQGLLQYSEEDCAFEEEIRPDG |
| | YNVYRSEKHRLPVSLSSAKQRQLYKNRGFLPLSHFLPMLPMVPEEPEDLRG |
| | HLESDMFSSPLETDSMDPFGLVTGLEAVRSPSFEK |
| 7. | RPLAFSDSSPLLQFGGQVRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 8. | RHPIPDSSPLLQWGDPIRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 9. | RHPIPDSSPLLQFGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 10. | RHPIPDSSPHVHYGWGDPIRLRHLYTSGPHGLSSCFLRIRADGVVDCARGQ |
| | SAHSLLEIKAVALRTVAIKGVHSVRYLCMGADGKMQGLLQYSEEDCAFEE |
| | EIRPDGYNVYRSEKHRLPVSLSSAKQRQLYKNRGFLPLSHFLPMLPMVPEE |
| | PEDLRGHLESDMFSSPLETDSMDPFGLVTGLEAVRSPSFEK |
| 11. | RPLAFSDAGPLLQWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 12. | RPLAFSDAGPLLQFGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 13. | RPLAFSDAGPLLQFGGQVRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 14. | RHPIPDSSPHVHYGGQVRLRHLYTSGPHGLSSCFLRIRADGVVDCARGQSA |
| | HSLLEIKAVALRTVAIKGVHSVRYLCMGADGKMQGLLQYSEEDCAFEEEI |
| | RPDGYNVYRSEKHRLPVSLSSAKQRQLYKNRGFLPLSHFLPMLPMVPEEPE |
| | DLRGHLESDMFSSPLETDSMDPFGLVTGLEAVRSPSFEK |
| 15. | RPLAFSDAGPHVHYGGQVRLRHLYTSGPHGLSSCFLRIRADGVVDCARGQ |
| | SAHSLLEIKAVALRTVAIKGVHSVRYLCMGADGKMQGLLQYSEEDCAFEE |
| | EIRPDGYNVYRSEKHRLPVSLSSAKQRQLYKNRGFLPLSHFLPMLPMVPEE |
| | PEDLRGHLESDMFSSPLETDSMDPFGLVTGLEAVRSPSFEK |
| 16. | RPLAFSDAGPHVHWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 17. | RPLAFSDAGPHVGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 18. | RPLAFSDAGPHYGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 19. | RPLAFSDAGPVYGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 20. | RPLAFSDAGPVHGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 21. | RPLAFSDAGPVHYWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 22. | RPLAFSDAGPHVHGWGDPIRLRHLYTSGPHGLSSCFLRIRADGVVDCARGQ |
| | SAHSLLEIKAVALRTVAIKGVHSVRYLCMGADGKMQGLLQYSEEDCAFEE |
| | EIRPDGYNVYRSEKHRLPVSLSSAKQRQLYKNRGFLPLSHFLPMLPMVPEE |
| | PEDLRGHLESDMFSSPLETDSMDPFGLVTGLEAVRSPSFEK |
| 23. | RPLAFSDAGPHHGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 24. | RPLAFSDAGPHHYWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 25. | RPLAFSDAGPHVYWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 26. | RPLAFSDSSPLVHWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 27. | RPLAFSDSSPHVHWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 28. | RPLAFSDAGPHVWGDPIRLRHLYTSGPHGLSSCFLRIRADGVVDCARGQSA |
| | HSLLEIKAVALRTVAIKGVHSVRYLCMGADGKMQGLLQYSEEDCAFEEEI |
| | RPDGYNVYRSEKHRLPVSLSSAKQRQLYKNRGFLPLSHFLPMLPMVPEEPE |
| | DLRGHLESDMFSSPLETDSMDPFGLVTGLEAVRSPSFEK |
| 29. | RPLAFSDAGPHVHYWGDPIRLRHLYTSGPHGLSSCFLRIRADGVVDCARGQ |
| | SAHSLLEIKAVALRTVAIKGVHSVRYLCMGADGKMQGLLQYSEEDCAFEE |
| | EIRPDGYNVYRSEKHRLPVSLSSAKQRQLYKNRGFLPLSHFLPMLPMVPEE |
| | PEDLRGHLESDMFSSPLETDSMDPFGLVTGLEAVRSPSFEK |
| 30. | RPLAFSDAGPHVHYAWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 31. | RHPIPDSSPLLQFGAQVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 32. | RHPIPDSSPLLQFGDQVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 33. | RHPIPDSSPLLQFGPQVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 34. | RHPIPDSSPLLQFGGAVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 35. | RHPIPDSSPLLQFGGEVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 36. | RHPIPDSSPLLQFGGNVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 37. | RHPIPDSSPLLQFGGQARLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 38. | RHPIPDSSPLLQFGGQIRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 39. | RHPIPDSSPLLQFGGQTRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 40. | RHPIPDSSPLLQFGWGQPVRLRHLYTSGPHGLSSCFLRIRADGVVDCARGQ |
| | SAHSLLEIKAVALRTVAIKGVHSVRYLCMGADGKMQGLLQYSEEDCAFEE |
| | EIRPDGYNVYRSEKHRLPVSLSSAKQRQLYKNRGFLPLSHFLPMLPMVPEE |
| | PEDLRGHLESDMFSSPLETDSMDPFGLVTGLEAVRSPSFEK |
| 41. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPEPPGI |
| | LAPQPPDVGSSDPLSMVGPSQGRSPSYAS |
| 42. | HPIPDSSPLLQFGGQVRLRHLYTSGPHGLSSCFLRIRADGVVDCARGQSAHS |
| | LLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPEPPGILAPQP |
| | PDVGSSDPLSMVGPSQGRSPSYAS |
| 43. | RPLAFSDAGPHVHYGGDIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 44. | RPLAFSDAGPHVHYGWGDPIRQRYLYTDDAQQTEAHLEIREDGTVGGAAD |
| | QSPESLLQLKALKPGVIQILGVKTSRFLCQRPDGALYGSLHFDPEACSFREL |
| | LLEDGYNVYQSEAHGLPLHLPGNKSPHRDPAPRGPARFLPLPGLPPALPEPP |
| | GILAPQPPDVGSSDPLSMVGPSQGRSPSYAS |
| 45. | HPIPDSSPLLQFGGQVRQRYLYTDDAQQTEAHLEIREDGTVGGAADQSPES |
| | LLQLKALKPGVIQILGVKTSRFLCQRPDGALYGSLHFDPEACSFRELLLEDG |
| | YNVYQSEAHGLPLHLPGNKSPHRDPAPRGPARFLPLPGLPPALPMVPEEPE |
| | DLRGHLESDMFSSPLETDSMDPFGLVTGLEAVRSPSFEK |
| 46. | RPLAFSDAGPHVHYGWGDPIRQRYLYTDDAQQTEAHLEIREDGTVGGAAD |
| | QSPESLLQLKALKPGVIQILGVKTSRFLCQRPDGALYGSLHFDPEACSFREL |
| | LLEDGYNVYQSEAHGLPLHLPGNKSPHRDPAPRGPARFLPLPGLPPALPEPP |
| | GILAPQPPDVGSSDPLSMVGPSQGRSPSYASPMVPEEPEDLRGHLESDMFSS |
| | PLETDSMDPFGLVTGLEAVRSPSFEK |
| 47. | HPIPDSSPLLQWGDPIRLRHLYTSGPHGLSSCFLRIRADGVVDCARGQSAHS |
| | LLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 48. | RDSSPLLQFGGQVRLRHLYTSGPHGLSSCFLRIRADGVVDCARGQSAHSLL |
| | EIKAVALRTVAIKGVHSVRYLCMGADGKMQGLLQYSEEDCAFEEEIRPDG |
| | YNVYRSEKHRLPVSLSSAKQRQLYKNRGFLPLSHFLPMLPMVPEEPEDLRG |
| | HLESDMFSSPLETDSMDPFGLVTGLEAVRSPSFEK |
| 49. | RPLAFSDSSPLLQFGGQVRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 50. | RHPIPDSSPLLQFGDQVRLRHLYTSGPHGLSSCFLRIRADGVVDCARGQSAH |
| | SLLEIKAVALRTVAIKGVHSVRYLCMGADGKMQGLLQYSEEDCAFEEEILE |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 51. | RHPIPDSSPLLQFGGNVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 52. | RDSSPLLQWGDPIRLRHLYTSGPHGLSSCFLRIRADGVVDCARGQSAHSLL |
| | EIKAVALRTVAIKGVHSVRYLCMGADGKMQGLLQYSEEDCAFEEEIRPDG |
| | YNVYRSEKHRLPVSLSSAKQRQLYKNRGFLPLSHFLPMLPMVPEEPEDLRG |
| | HLESDMFSSPLETDSMDPFGLVTGLEAVRSPSFEK |
| 53. | MDSSPLVHYGWGDPIRLRHLYTSGPHGLSSCFLRIRADGVVDCARGQSAHS |
| | LLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 54. | RPLAFSDAGPLLQWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 55. | RPLAFSDAGPHYGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 56. | RPLAFSDAGPVYGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 57. | RPLAFSDAGPVHGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 58. | RPLAFSDAGPVHYWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 59. | RPLAFSDAGPHHGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 60. | RPLAFSDAGPHHYWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 61. | RPLAFSDAGPHVGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 62. | RPLAFSDAGPHVYWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 63. | RPLAFSDAGPHVHWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 64. | RPLAFSDSSPLVHWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 65. | RPLAFSDSSPHVHWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 66. | RPLAFSDAGPHLQWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 67. | RPLAFSDAGPHVWGDPIRLRHLYTSGPHGLSSCFLRIRADGVVDCARGQSA |
| | HSLLEIKAVALRTVAIKGVHSVRYLCMGADGKMQGLLQYSEEDCAFEEEI |
| | RPDGYNVYRSEKHRLPVSLSSAKQRQLYKNRGFLPLSHFLPMLPMVPEEPE |
| | DLRGHLESDMFSSPLETDSMDPFGLVTGLEAVRSPSFEK |
| 68. | RPLAFSDAGPHVHYWGDPIRLRHLYTSGPHGLSSCFLRIRADGVVDCARGQ |
| | SAHSLLEIKAVALRTVAIKGVHSVRYLCMGADGKMQGLLQYSEEDCAFEE |
| | EIRPDGYNVYRSEKHRLPVSLSSAKQRQLYKNRGFLPLSHFLPMLPMVPEE |
| | PEDLRGHLESDMFSSPLETDSMDPFGLVTGLEAVRSPSFEK |
| 69. | RDSSPLVHYGWGDPIRLRHLYTSGPHGLSSCFLRIRADGVVDCARGQSAHS |
| | LLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 70. | MRDSSPLVHYGWGDPIRLRHLYTSGPHGLSSCFLRIRADGVVDCARGQSA |
| | HSLLEIKAVALRTVAIKGVHSVRYLCMGADGKMQGLLQYSEEDCAFEEEI |
| | RPDGYNVYRSEKHRLPVSLSSAKQRQLYKNRGFLPLSHFLPMLPMVPEEPE |
| | DLRGHLESDMFSSPLETDSMDPFGLVTGLEAVRSPSFEK |
| 71. | HPIPDSSPLLQFGGQVRQRYLYTDDAQQTEAHLEIREDGTVGGAADQSPES |
| | LLQLKALKPGVIQILGVKTSRFLCQRPDGALYGSLHFDPEACSFRELLLEDG |
| | YNVYQSEAHSLPLHLPGNKSPHRDPAPRGPARFLPLPGLPPALPEPPGILAP |
| | QPPDVGSSDPLSMVGPSQGRSPSYAS |
| 72. | HPIPDSSPLLQFGGQVRQRYLYTDDAQQTEAHLEIREDGTVGGAADQSPES |
| | LLQLKALKPGVIQILGVKTSRFLCQRPDGALYGSLHFDPEACSFRELLLEDG |
| | YNVYQSEAHGLPLHLPGNKSPHRDPAPRGPARFLPLPGLPPAPPEPPGILAP |
| | QPPDVGSSDPLSMVGPSQGRSPSYAS |
| 73. | HPIPDSSPLLQFGGQVRQRYLYTDDAQQTEAHLEIREDGTVGGAADQSPES |
| | LLQLKALKPGVIQILGVKTSRFLCQRPDGALYGSLHFDPEACSFRELLLEDG |
| | YNVYQSEAHGLPLHLPGNKSPHRDPAPRGPARFLPLPGLPPALPEPPGILAP |
| | QPPDVGSSDPLSMVVQDELQGVGGEGCHMHPENCKTLLTDIDRTHTEKPV |
| | WDGITGE |
| 74. | RDAGPHVHYGWGDPIRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 75. | RVHYGWGDPIRLRHLYTSGPHGLSSCFLRIRADGVVDCARGQSAHSLLEIK |
| | AVALRTVAIKGVHSVRYLCMGADGKMQGLLQYSEEDCAFEEEIRPDGYN |
| | VYRSEKHRLPVSLSSAKQRQLYKNRGFLPLSHFLPMLPMVPEEPEDLRGHL |
| | ESDMFSSPLETDSMDPFGLVTGLEAVRSPSFEK |
| 76. | RGDPIRLRHLYTSGPHGLSSCFLRIRADGVVDCARGQSAHSLLEIKAVALRT |
| | VAIKGVHSVRYLCMGADGKMQGLLQYSEEDCAFEEEIRPDGYNVYRSEKH |
| | RLPVSLSSAKQRQLYKNRGFLPLSHFLPMLPMVPEEPEDLRGHLESDMFSSP |
| | LETDSMDPFGLVTGLEAVRSPSFEK |
| 77. | RRLRHLYTSGPHGLSSCFLRIRADGVVDCARGQSAHSLLEIKAVALRTVAI |
| | KGVHSVRYLCMGADGKMQGLLQYSEEDCAFEEEIRPDGYNVYRSEKHRL |
| | PVSLSSAKQRQLYKNRGFLPLSHFLPMLPMVPEEPEDLRGHLESDMFSSPLE |
| | TDSMDPFGLVTGLEAVRSPSFEK |
| 78. | RAGPHVHYGWGDPIRLRHLYTSGPHGLSSCFLRIRADGVVDCARGQSAHS |
| | LLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 79. | RGPHVHYGWGDPIRLRHLYTSGPHGLSSCFLRIRADGVVDCARGQSAHSLL |
| | EIKAVALRTVAIKGVHSVRYLCMGADGKMQGLLQYSEEDCAFEEEIRPDG |
| | YNVYRSEKHRLPVSLSSAKQRQLYKNRGFLPLSHFLPMLPMVPEEPEDLRG |
| | HLESDMFSSPLETDSMDPFGLVTGLEAVRSPSFEK |
| 80. | RPHVHYGWGDPIRLRHLYTSGPHGLSSCFLRIRADGVVDCARGQSAHSLLE |
| | IKAVALRTVAIKGVHSVRYLCMGADGKMQGLLQYSEEDCAFEEEIRPDGY |
| | NVYRSEKHRLPVSLSSAKQRQLYKNRGFLPLSHFLPMLPMVPEEPEDLRGH |
| | LESDMFSSPLETDSMDPFGLVTGLEAVRSPSFEK |
| 81. | RHVHYGWGDPIRLRHLYTSGPHGLSSCFLRIRADGVVDCARGQSAHSLLEI |
| | KAVALRTVAIKGVHSVRYLCMGADGKMQGLLQYSEEDCAFEEEIRPDGY |
| | NVYRSEKHRLPVSLSSAKQRQLYKNRGFLPLSHFLPMLPMVPEEPEDLRGH |
| | LESDMFSSPLETDSMDPFGLVTGLEAVRSPSFEK |
| 82. | RPLAFSAAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 83. | RPLAFSDAAPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 84. | RPLAFSDAGAHVHYGWGDPIRLRHLYTSGPHGLSSCFLRIRADGVVDCAR |
| | GQSAHSLLEIKAVALRTVAIKGVHSVRYLCMGADGKMQGLLQYSEEDCAF |
| | EEEIRPDGYNVYRSEKHRLPVSLSSAKQRQLYKNRGFLPLSHFLPMLPMVP |
| | EEPEDLRGHLESDMFSSPLETDSMDPFGLVTGLEAVRSPSFEK |
| 85. | RPLAFSDAGPHVHYGAGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 86. | RPLAFSDAGPHVHYGWGAPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 87. | RPLAFSDAGPHVHYGWGDAICARGQSAHSLLEIKAVALRTVAIKGVHSVR |
| | YLCMGADGKMQGLLQYSEEDCAFEEEIRPDGYNVYRSEKHRLPVSLSSAK |
| | QRQLYKNRGFLPLSHFLPMLPMVPEEPEDLRGHLESDMFSSPLETDSMDPF |
| | GLVTGLEAVRSPSFEK |
| 88. | RPLAFSDAGPHVHYGWGDPIRLRHLYTSGPAGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLAHFLPMLPMVPE |
| | EPEDLRGHLESDMFSSPLETDSMDPFGLVTGLEAVRSPSFEK |
| 89. | RPLAFSDAGPHVHYGWGDPIRLRHLYTSGPAGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSAFLPMLPMVPE |
| | EPEDLRGHLESDMFSSPLETDSMDPFGLVTGLEAVRSPSFEK |
| 90. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAAQAQLYKNRGFLPLSHFLPMLPMVPE |
| | EPEDLRGHLESDMFSSPLETDSMDPFGLVTGLEAVRSPSFEK |
| 91. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAAQRQLYKNRGFLPLAHFLPMLPMVPE |
| | EPEDLRGHLESDMFSSPLETDSMDPFGLVTGLEAVRSPSFEK |
| 92. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAAQRQLYKNRGFLPLSAFLPMLPMVPE |
| | EPEDLRGHLESDMFSSPLETDSMDPFGLVTGLEAVRSPSFEK |
| 93. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQAQLYKNRGFLPLAHFLPMLPMVPE |
| | EPEDLRGHLESDMFSSPLETDSMDPFGLVTGLEAVRSPSFEK |
| 94. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLAAFLPMLPMVPE |
| | EPEDLRGHLESDMFSSPLETDSMDPFGLVTGLEAVRSPSFEK |
| 95. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAAQRQLYKNRGFLPLSAFLPMLPMVPE |
| | EPEDLRGHLESDMFSSPLETDSMDPFGLVTGLEAVRSPSFEK |
| 96. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAAQAQLYKNRGFLPLAHFLPMLPMVPE |
| | EPEDLRGHLESDMFSSPLETDSMDPFGLVTGLEAVRSPSFEK |
| 97. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAAQAQLYKNRGFLPLSAFLPMLPMVPE |
| | EPEDLRGHLESDMFSSPLETDSMDPFGLVTGLEAVRSPSFEK |
| 98. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAAQAQLYKNRGFLPLAAFLPMLPMVPE |
| | EPEDLRGHLESDMFSSPLETDSMDPFGLVTGLEAVRSPSFEK |
| 138. | DSSPLLQFGGQVRLRHLYTSGPHGLSSCFLRIRADGVVDCARGQSAHSLLEI |
| | KAVALRTVAIKGVHSVRYLCMGADGKMQGLLQYSEEDCAFEEEIRPDGY |
| | NVYRSEKHRLPVSLSSAKQRQLYKNRGFLPLSHFLPMLPMVPEEPEDLRGH |
| | LESDMFSSPLETDSMDPFGLVTGLEAVRSPSFEK |
| 139. | RPLAFSDASPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 140. | RPLAFSDSSPLVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 141. | DSSPLVHYGWGDPIRLRHLYTSGPHGLSSCFLRIRADGVVDCARGQSAHSL |
| | LEIKAVALRTVAIKGVHSVRYLCMGADGKMQGLLQYSEEDCAFEEEIRPD |
| | GYNVYRSEKHRLPVSLSSAKQRQLYKNRGFLPLSHFLPMLPMVPEEPEDLR |
| | GHLESDMFSSPLETDSMDPFGLVTGLEAVRSPSFEK |
| 142. | RHPIPDSSPLLQFGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 143. | RHPIPDSSPLLQWGDPIRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 144. | RPLAFSDAGPLLQFGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIRPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 145. | RHPIPDSSPHVHYGWGDPIRLRHLYTSGPHGLSSCFLRIRADGVVDCARGQ |
| | SAHSLLEIKAVALRTVAIKGVHSVRYLCMGADGKMQGLLQYSEEDCAFEE |
| | EIRPDGYNVYRSEKHRLPVSLSSAKQRQLYKNRGFLPLSHFLPMLPMVPEE |
| | PEDLRGHLESDMFSSPLETDSMDPFGLVTGLEAVRSPSFEK |
| 146. | RPLAFSDAGPLLQFGGQVRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 147. | RHPIPDSSPHVHYGGQVRLRHLYTSGPHGLSSCFLRIRADGVVDCARGQSA |
| | HSLLEIKAVALRTVAIKGVHSVRYLCMGADGKMQGLLQYSEEDCAFEEEI |
| | RPDGYNVYRSEKHRLPVSLSSAKQRQLYKNRGFLPLSHFLPMLPMVPEEPE |
| | DLRGHLESDMFSSPLETDSMDPFGLVTGLEAVRSPSFEK |
| 148. | RDSSPLLQFGGQVRLRHLYTSGPHGLSSCFLRIRADGVVDCARGQSAHSLL |
| | EIKAVALRTVAIKGVHSVRYLCMGADGKMQGLLQYSEEDCAFEEEIRPDG |
| | YNVYRSEKHRLPVSLSSAKQRQLYKNRGFLPLSHFLPMLPMVPEEPEDLRG |
| | HLESDMFSSPLETDSMDPFGLVTGLEAVRSPSFEK |
| 149. | RPLAFSDSSPLLQFGGQVRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 150. | RHPIPDSSPLLQFGAQVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 151. | RHPIPDSSPLLQFGDQVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 152. | RHPIPDSSPLLQFGPQVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 153. | RHPIPDSSPLLQFGGAVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 154. | RHPIPDSSPLLQFGGEVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 155. | RHPIPDSSPLLQFGGNVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 156. | RHPIPDSSPLLQFGGQARLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 157. | RHPIPDSSPLLQFGGQIRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 158. | RHPIPDSSPLLQFGGQTRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 159. | RHPIPDSSPLLQFGWGQPVRLRHLYTSGPHGLSSCFLRIRADGVVDCARGQ |
| | SAHSLLEIKAVALRTVAIKGVHSVRYLCMGADGKMQGLLQYSEEDCAFEE |
| | EIRPDGYNVYRSEKHRLPVSLSSAKQRQLYKNRGFLPLSHFLPMLPMVPEE |
| | PEDLRGHLESDMFSSPLETDSMDPFGLVTGLEAVRSPSFEK |
| 160. | HPIPDSSPLLQFGGQVRLRHLYTSGPHGLSSCFLRIRADGVVDCARGQSAHS |
| | LLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 161. | DSSPLLQFGGQVRLRHLYTSGPHGLSSCFLRIRADGVVDCARGQSAHSLLEI |
| | KAVALRTVAIKGVHSVRYLCMGADGKMQGLLQYSEEDCAFEEEIRPDGY |
| | NVYRSEKHRLPVSLSSAKQRQLYKNRGFLPLSHFLPMLPMVPEEPEDLRGH |
| | LESDMFSSPLETDSMDPFGLVTGLEAVRSPSFEK |
| 162. | HPIPDSSPLLQWGDPIRLRHLYTSGPHGLSSCFLRIRADGVVDCARGQSAHS |
| | LLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 163. | HPIPDSSPLLQFGWGDPIRLRHLYTSGPHGLSSCFLRIRADGVVDCARGQSA |
| | HSLLEIKAVALRTVAIKGVHSVRYLCMGADGKMQGLLQYSEEDCAFEEEI |
| | RPDGYNVYRSEKHRLPVSLSSAKQRQLYKNRGFLPLSHFLPMLPMVPEEPE |
| | DLRGHLESDMFSSPLETDSMDPFGLVTGLEAVRSPSFEK |
| 164. | HPIPDSSPHVHYGWGDPIRLRHLYTSGPHGLSSCFLRIRADGVVDCARGQS |
| | AHSLLEIKAVALRTVAIKGVHSVRYLCMGADGKMQGLLQYSEEDCAFEEE |
| | IRPDGYNVYRSEKHRLPVSLSSAKQRQLYKNRGFLPLSHFLPMLPMVPEEP |
| | EDLRGHLESDMFSSPLETDSMDPFGLVTGLEAVRSPSFEK |
| 165. | HPIPDSSPHVHYGGQVRLRHLYTSGPHGLSSCFLRIRADGVVDCARGQSAH |
| | SLLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 166. | DAGPHVHYGWGDPIRLRHLYTSGPHGLSSCFLRIRADGVVDCARGQSAHS |
| | LLEIKAVALRTVAIKGVHSVRYLCMGADGKMQGLLQYSEEDCAFEEEIRP |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 167. | VHYGWGDPIRLRHLYTSGPHGLSSCFLRIRADGVVDCARGQSAHSLLEIKA |
| | VALRTVAIKGVHSVRYLCMGADGKMQGLLQYSEEDCAFEEEIRPDGYNV |
| | YRSEKHRLPVSLSSAKQRQLYKNRGFLPLSHFLPMLPMVPEEPEDLRGHLE |
| | SDMFSSPLETDSMDPFGLVTGLEAVRSPSFEK |
| 168. | RLRHLYTSGPHGLSSCFLRIRADGVVDCARGQSAHSLLEIKAVALRTVAIK |
| | GVHSVRYLCMGADGKMQGLLQYSEEDCAFEEEIRPDGYNVYRSEKHRLP |
| | VSLSSAKQRQLYKNRGFLPLSHFLPMLPMVPEEPEDLRGHLESDMFSSPLET |
| | DSMDPFGLVTGLEAVRSPSFEK |
| 188. | PHGLSSCFLRIRADGVVDCARGQSAHSLLEIKAVALRTVAIKGVHSVRYLC |
| | MGADGKMQGLLQYSEEDCAFEEEIRPDGYNVYRSEKHRLPVSLSSAKQRQ |
| | LYKNRGFLPLSHFLPMLPMVPEEPEDLRGHLESDMFSSPLETDSMDPFGLV |
| | TGLEAVRSPSFEK |
| 192. | MDSSPLLQWGDPIRLRHLYTSGPHGLSSCFLRIRADGVVDCARGQSAHSLL |
| | EIKAVALRTVAIKGVHSVRYLCMGADGKMQGLLQYSEEDCAFEEEIRPDG |
| | YNVYRSEKHRLPVSLSSAKQRQLYKNRGFLPLSHFLPMLPMVPEEPEDLRG |
| | HLESDMFSSPLETDSMDPFGLVTGLEAVRSPSFEK |
| 193. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEILPDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 194. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEIREDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 195. | RPLAFSDAGPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEILCDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 196. | RPLAFSDAGPHVHYGWGDPIRQRHLYTSGPHGLSSCFLRIRADGVVDCAR |
| | GQSAHSLLEIKAVALRTVAIKGVHSVRYLCMGADGKMQGLLQYSEEDCAF |
| | EEEILEDGYNVYRSEKHRLPVSLSSAKQRQLYKNRGFLPLSHFLPMLPMVP |
| | EEPEDLRGHLESDMFSSPLETDSMDPFGLVTGLEAVRSPSFEK |
| 197. | RDSSPLVHYGWGDPIRLRHLYTSGPHGLSSCFLRIRADGVVDCARGQSAHS |
| | LLEIKAVALRTVAIKGVHSVRYLCMGADGKMQGLLQYSEEDCAFEEEILE |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 198. | RPLAFSDSSPLVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEILEDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 199. | RPLAFSDASPHVHYGWGDPIRLRHLYTSGPHGLSSCFLRIRADGVVDCARG |
| | QSAHSLLEIKAVALRTVAIKGVHSVRYLCMGADGKMQGLLQYSEEDCAFE |
| | EEILEDGYNVYRSEKHRLPVSLSSAKQRQLYKNRGFLPLSHFLPMLPMVPE |
| | EPEDLRGHLESDMFSSPLETDSMDPFGLVTGLEAVRSPSFEK |
| 200. | RDSSPLLQWGDPIRLRHLYTSGPHGLSSCFLRIRADGVVDCARGQSAHSLL |
| | EIKAVALRTVAIKGVHSVRYLCMGADGKMQGLLQYSEEDCAFEEEILEDG |
| | YNVYRSEKHRLPVSLSSAKQRQLYKNRGFLPLSHFLPMLPMVPEEPEDLRG |
| | HLESDMFSSPLETDSMDPFGLVTGLEAVRSPSFEK |
| 201. | RHPIPDSSPLLQFGDQVRLRHLYTSGPHGLSSCFLRIRADGVVDCARGQSAH |
| | SLLEIKAVALRTVAIKGVHSVRYLCMGADGKMQGLLQYSEEDCAFEEEILE |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 202. | RDSSPLLQFGGQVRLRHLYTSGPHGLSSCFLRIRADGVVDCARGQSAHSLL |
| | EIKAVALRTVAIKGVHSVRYLCMGADGKMQGLLQYSEEDCAFEEEILEDG |
| | YNVYRSEKHRLPVSLSSAKQRQLYKNRGFLPLSHFLPMLPMVPEEPEDLRG |
| | HLESDMFSSPLETDSMDPFGLVTGLEAVRSPSFEK |
| 203. | RHPIPDSSPLLQFGGQVRLRHLYTSGPHGLSSCFLRIRADGVVDCARGQSAH |
| | SLLEIKAVALRTVAIKGVHSVRYLCMGADGKMQGLLQYSEEDCAFEEEILE |
| | DGYNVYRSEKHRLPVSLSSAKQRQLYKNRGFLPLSHFLPMLPMVPEEPEDL |
| | RGHLESDMFSSPLETDSMDPFGLVTGLEAVRSPSFEK |
| 204. | MRDSSPLVHYGWGDPIRLRHLYTSGPHGLSSCFLRIRADGVVDCARGQSA |
| | HSLLEIKAVALRTVAIKGVHSVRYLCMGADGKMQGLLQYSEEDCAFEEEIL |
| | EDGYNVYRSEKHRLPVSLSSAKQRQLYKNRGFLPLSHFLPMLPMVPEEPED |
| | LRGHLESDMFSSPLETDSMDPFGLVTGLEAVRSPSFEK |

In one embodiment, the peptide sequence comprises an amino acid sequence set forth in SEQ ID NO:1. In another embodiment, the peptide sequence comprises an amino acid sequence set forth in SEQ ID NO:2. In other embodiments, the peptide sequence comprises an amino acid sequence set forth in SEQ ID NO:3. In one embodiment, the peptide sequence comprises an amino acid sequence set forth in SEQ ID NO:4. In another embodiment, the peptide sequence comprises an amino acid sequence set forth in SEQ ID NO:5. In other embodiments, the peptide sequence comprises an amino acid sequence set forth in SEQ ID NO:6. In one embodiment, the peptide sequence comprises an amino acid sequence set forth in SEQ ID NO:7. In another embodiment, the peptide sequence comprises an amino acid sequence set forth in SEQ ID NO:8. In other embodiments, the peptide sequence comprises an amino acid sequence set forth in SEQ ID NO:9. In one embodiment, the peptide sequence comprises an amino acid sequence set forth in SEQ ID NO:10. In another embodiment, the peptide sequence comprises an amino acid sequence set forth in SEQ ID NO:11. In other embodiments, the peptide sequence comprises an amino acid sequence set forth in SEQ ID NO:12. In one embodiment, the peptide sequence comprises an amino acid sequence set forth in SEQ ID NO:13. In another embodiment, the peptide sequence comprises an amino acid sequence set forth in SEQ ID NO:14. In other embodiments, the peptide sequence comprises an amino acid sequence set forth in SEQ ID NO:15. In one embodiment, the peptide sequence comprises an amino acid sequence set forth in SEQ ID NO:16. In another embodiment, the peptide sequence comprises an amino acid sequence set forth in SEQ ID NO:17. In other embodiments, the peptide sequence comprises an amino acid sequence set forth in SEQ ID NO:18. In one embodiment, the peptide sequence comprises an amino acid sequence set forth in SEQ ID NO:19. In another embodiment, the peptide sequence comprises an amino acid sequence set forth in SEQ ID NO:20. In other embodiments, the peptide sequence comprises an amino acid sequence set forth in SEQ ID NO:21. In one embodiment, the peptide sequence comprises an amino acid sequence set forth in SEQ ID NO:22. In another embodiment, the peptide sequence comprises an amino acid sequence set forth in SEQ ID NO:23. In other embodiments, the peptide sequence comprises an amino acid sequence set forth in SEQ ID NO:24. In one embodiment, the peptide sequence comprises an amino acid sequence set forth in SEQ ID NO:25. In another embodiment, the peptide sequence comprises an amino acid sequence set forth in SEQ ID NO:26. In other embodiments, the peptide sequence comprises an amino acid sequence set forth in SEQ ID NO:27. In one embodiment, the peptide sequence comprises an amino acid sequence set forth in SEQ ID NO:28. In another embodiment, the peptide sequence comprises an amino acid sequence set forth in SEQ ID NO:29. In other embodiments, the peptide sequence comprises an amino acid sequence set forth in SEQ ID NO:30. In one embodiment, the peptide sequence comprises an amino acid sequence set forth in SEQ ID NO:31. In another embodiment, the peptide sequence comprises an amino acid sequence set forth in SEQ ID NO:32. In other embodiments, the peptide sequence comprises an amino acid sequence set forth in SEQ ID NO:33. In one embodiment, the peptide sequence comprises an amino acid sequence set forth in SEQ ID NO:34. In another embodiment, the peptide sequence comprises an amino acid sequence set forth in SEQ ID NO:35. In other embodiments, the peptide sequence comprises an amino acid sequence set forth in SEQ ID NO:36. In one embodiment, the peptide sequence comprises an amino acid sequence set forth in SEQ ID NO:37. In another embodiment, the peptide sequence comprises an amino acid sequence set forth in SEQ ID NO:38. In other embodiments, the peptide sequence comprises an amino acid sequence set forth in SEQ ID NO:39. In one embodiment, the peptide sequence comprises an amino acid sequence set forth in SEQ ID NO:40. In another embodiment, the peptide sequence comprises an amino acid sequence set forth in SEQ ID NO:41. In other embodiments, the peptide sequence comprises an amino acid sequence set forth in SEQ ID NO:42. In one embodiment, the peptide sequence comprises an amino acid sequence set forth in SEQ ID NO:43. In another embodiment, the peptide sequence comprises an amino acid sequence set forth in SEQ ID NO:44. In other embodiments, the peptide sequence comprises an amino acid sequence set forth in SEQ ID NO:45. In one embodiment, the peptide sequence comprises an amino acid sequence set forth in SEQ ID NO:46. In another embodiment, the peptide sequence comprises an amino acid sequence set forth in SEQ ID NO:47. In other embodiments, the peptide sequence comprises an amino acid sequence set forth in SEQ ID NO:48. In one embodiment, the peptide sequence comprises an amino acid sequence set forth in SEQ ID NO:49. In another embodiment, the peptide sequence comprises an amino acid sequence set forth in SEQ ID NO:50. In other embodiments, the peptide sequence comprises an amino acid sequence set forth in SEQ ID NO:51. In one embodiment, the peptide sequence comprises an amino acid sequence set forth in SEQ ID NO:52. In another embodiment, the peptide sequence comprises an amino acid sequence set forth in SEQ ID NO:53. In other embodiments, the peptide sequence comprises an amino acid sequence set forth in SEQ ID NO:54. In one embodiment, the peptide sequence comprises an amino acid sequence set forth in SEQ ID NO:55. In another embodiment, the peptide sequence comprises an amino acid sequence set forth in SEQ ID NO:56. In other embodiments, the peptide sequence comprises an amino acid sequence set forth in SEQ ID NO:57. In one embodiment, the peptide sequence comprises an amino acid sequence set forth in SEQ ID NO:58. In another embodiment, the peptide sequence comprises an amino acid sequence set forth in SEQ ID NO:59. In other embodiments, the peptide sequence comprises an amino acid sequence set forth in SEQ ID NO:60. In one embodiment, the peptide sequence comprises an amino acid sequence set forth in SEQ ID NO:61. In another embodiment, the peptide sequence comprises an amino acid sequence set forth in SEQ ID NO:62. In other embodiments, the peptide sequence comprises an amino acid sequence set forth in SEQ ID NO:63. In one embodiment, the peptide sequence comprises an amino acid sequence set forth in SEQ ID NO:64. In another embodiment, the peptide sequence comprises an amino acid sequence set forth in SEQ ID NO:65. In other embodiments, the peptide sequence comprises an amino acid sequence set forth in SEQ ID NO:66. In one embodiment, the peptide sequence comprises an amino acid sequence set forth in SEQ ID NO:67. In another embodiment, the peptide sequence comprises an amino acid sequence set forth in SEQ ID NO:68. In other embodiments, the peptide sequence comprises an amino acid sequence set forth in SEQ ID NO:69. In one embodiment, the peptide sequence comprises an amino acid sequence set forth in SEQ ID NO:70. In another embodiment, the peptide sequence comprises an amino acid sequence set forth in SEQ ID NO:71. In other embodiments, the peptide sequence comprises an amino acid sequence set forth in SEQ ID NO:72. In one embodiment, the peptide sequence comprises an amino acid sequence set forth in SEQ ID NO:73. In another embodiment, the peptide sequence comprises an amino acid sequence set forth in SEQ ID NO:74. In other embodiments, the peptide sequence comprises an amino acid sequence set forth in SEQ ID NO:75. In one embodiment, the peptide sequence comprises an amino acid sequence set forth in SEQ ID NO:76. In another embodiment, the peptide sequence comprises an amino acid sequence set forth in SEQ ID NO:77. In other embodiments, the peptide sequence comprises an amino acid sequence set forth in SEQ ID NO:78. In one embodiment, the peptide sequence comprises an amino acid sequence set forth in SEQ ID NO:79. In another embodiment, the peptide sequence comprises an amino acid sequence set forth in SEQ ID NO:80. In other embodiments, the peptide sequence comprises an amino acid sequence set forth in SEQ ID NO:81. In one embodiment, the peptide sequence comprises an amino acid sequence set forth in SEQ ID NO:82. In another embodiment, the peptide sequence comprises an amino acid sequence set forth in SEQ ID NO:83. In other embodiments, the peptide sequence comprises an amino acid sequence set forth in SEQ ID NO:84. In one embodiment, the peptide sequence comprises an amino acid sequence set forth in SEQ ID NO:85. In another embodiment, the peptide sequence comprises an amino acid sequence set forth in SEQ ID NO:86. In other embodiments, the peptide sequence comprises an amino acid sequence set forth in SEQ ID NO:87. In one embodiment, the peptide sequence comprises an amino acid sequence set forth in SEQ ID NO:88. In another embodiment, the peptide sequence comprises an amino acid sequence set forth in SEQ ID NO:89. In other embodiments, the peptide sequence comprises an amino acid sequence set forth in SEQ ID NO:90. In one embodiment, the peptide sequence comprises an amino acid sequence set forth in SEQ ID NO:91. In another embodiment, the peptide sequence comprises an amino acid sequence set forth in SEQ ID NO:92. In other embodiments, the peptide sequence comprises an amino acid sequence set forth in SEQ ID NO:93. In one embodiment, the peptide sequence comprises an amino acid sequence set forth in SEQ ID NO:94. In another embodiment, the peptide sequence comprises an amino acid sequence set forth in SEQ ID NO:95. In other embodiments, the peptide sequence comprises an amino acid sequence set forth in SEQ ID NO:96. In one embodiment, the peptide sequence comprises an amino acid sequence set forth in SEQ ID NO:97. In another embodiment, the peptide sequence comprises an amino acid sequence set forth in SEQ ID NO:98. In other embodiments, the peptide sequence comprises an amino acid sequence set forth in SEQ ID NO:138. In one embodiment, the peptide sequence comprises an amino acid sequence set forth in SEQ ID NO:139. In another embodiment, the peptide sequence comprises an amino acid sequence set forth in SEQ ID NO:140. In other embodiments, the peptide sequence comprises an amino acid sequence set forth in SEQ ID NO:141. In one embodiment, the peptide sequence comprises an amino acid sequence set forth in SEQ ID NO:142. In another embodiment, the peptide sequence comprises an amino acid sequence set forth in SEQ ID NO:143. In other embodiments, the peptide sequence comprises an amino acid sequence set forth in SEQ ID NO:144. In one embodiment, the peptide sequence comprises an amino acid sequence set forth in SEQ ID NO:145. In another embodiment, the peptide sequence comprises an amino acid sequence set forth in SEQ ID NO:146. In other embodiments, the peptide sequence comprises an amino acid sequence set forth in SEQ ID NO:147. In one embodiment, the peptide sequence comprises an amino acid sequence set forth in SEQ ID NO:148. In another embodiment, the peptide sequence comprises an amino acid sequence set forth in SEQ ID NO:149. In other embodiments, the peptide sequence comprises an amino acid sequence set forth in SEQ ID NO:150. In one embodiment, the peptide sequence comprises an amino acid sequence set forth in SEQ ID NO:151. In another embodiment, the peptide sequence comprises an amino acid sequence set forth in SEQ ID NO:152. In other embodiments, the peptide sequence comprises an amino acid sequence set forth in SEQ ID NO:153. In one embodiment, the peptide sequence comprises an amino acid sequence set forth in SEQ ID NO:154. In another embodiment, the peptide sequence comprises an amino acid sequence set forth in SEQ ID NO:155. In other embodiments, the peptide sequence comprises an amino acid sequence set forth in SEQ ID NO:156. In one embodiment, the peptide sequence comprises an amino acid sequence set forth in SEQ ID NO:157. In another embodiment, the peptide sequence comprises an amino acid sequence set forth in SEQ ID NO:158. In other embodiments, the peptide sequence comprises an amino acid sequence set forth in SEQ ID NO:159. In one embodiment, the peptide sequence comprises an amino acid sequence set forth in SEQ ID NO:160. In another embodiment, the peptide sequence comprises an amino acid sequence set forth in SEQ ID NO:161. In other embodiments, the peptide sequence comprises an amino acid sequence set forth in SEQ ID NO:162. In one embodiment, the peptide sequence comprises an amino acid sequence set forth in SEQ ID NO:163. In another embodiment, the peptide sequence comprises an amino acid sequence set forth in SEQ ID NO:164. In other embodiments, the peptide sequence comprises an amino acid sequence set forth in SEQ ID NO:165. In one embodiment, the peptide sequence comprises an amino acid sequence set forth in SEQ ID NO:166. In another embodiment, the peptide sequence comprises an amino acid sequence set forth in SEQ ID NO:167. In other embodiments, the peptide sequence comprises an amino acid sequence set forth in SEQ ID NO:168. In another embodiment, the peptide sequence comprises an amino acid sequence set forth in SEQ ID NO:192. In other embodiments, the peptide sequence comprises an amino acid sequence set forth in SEQ ID NO:193. In one embodiment, the peptide sequence comprises an amino acid sequence set forth in SEQ ID NO:194. In another embodiment, the peptide sequence comprises an amino acid sequence set forth in SEQ ID NO:195. In other embodiments, the peptide sequence comprises an amino acid sequence set forth in SEQ ID NO:196. In one embodiment, the peptide sequence comprises an amino acid sequence set forth in SEQ ID NO:197. In another embodiment, the peptide sequence comprises an amino acid sequence set forth in SEQ ID NO:198. In other embodiments, the peptide sequence comprises an amino acid sequence set forth in SEQ ID NO:199. In one embodiment, the peptide sequence comprises an amino acid sequence set forth in SEQ ID NO:200. In another embodiment, the peptide sequence comprises an amino acid sequence set forth in SEQ ID NO:201. In other embodiments, the peptide sequence comprises an amino acid sequence set forth in SEQ ID NO:202. In one embodiment, the peptide sequence comprises an amino acid sequence set forth in SEQ ID NO:203. In another embodiment, the peptide sequence comprises an amino acid sequence set forth in SEQ ID NO:204. In certain embodiments of the various peptide sequences provided herein, the R residue at the N-terminus is deleted.

In yet other embodiments, the peptide sequence consists of an amino acid sequence set forth in SEQ ID NO:1. In another embodiment, the peptide sequence consists of an amino acid sequence set forth in SEQ ID NO:2. In other embodiments, the peptide sequence consists of an amino acid sequence set forth in SEQ ID NO:3. In one embodiment, the peptide sequence consists of an amino acid sequence set forth in SEQ ID NO:4. In another embodiment, the peptide sequence consists of an amino acid sequence set forth in SEQ ID NO: 5. In other embodiments, the peptide sequence consists of an amino acid sequence set forth in SEQ ID NO:6. In one embodiment, the peptide sequence consists of an amino acid sequence set forth in SEQ ID NO:7. In another embodiment, the peptide sequence consists of an amino acid sequence set forth in SEQ ID NO:8. In other embodiments, the peptide sequence consists of an amino acid sequence set forth in SEQ ID NO:9. In one embodiment, the peptide sequence consists of an amino acid sequence set forth in SEQ ID NO:10. In another embodiment, the peptide sequence consists of an amino acid sequence set forth in SEQ ID NO:11. In other embodiments, the peptide sequence consists of an amino acid sequence set forth in SEQ ID NO:12. In one embodiment, the peptide sequence consists of an amino acid sequence set forth in SEQ ID NO:13. In another embodiment, the peptide sequence consists of an amino acid sequence set forth in SEQ ID NO:14. In other embodiments, the peptide sequence consists of an amino acid sequence set forth in SEQ ID NO:15. In one embodiment, the peptide sequence consists of an amino acid sequence set forth in SEQ ID NO:16. In another embodiment, the peptide sequence consists of an amino acid sequence set forth in SEQ ID NO:17. In other embodiments, the peptide sequence consists of an amino acid sequence set forth in SEQ ID NO:18. In one embodiment, the peptide sequence consists of an amino acid sequence set forth in SEQ ID NO:19. In another embodiment, the peptide sequence consists of an amino acid sequence set forth in SEQ ID NO:20. In other embodiments, the peptide sequence consists of an amino acid sequence set forth in SEQ ID NO:21. In one embodiment, the peptide sequence consists of an amino acid sequence set forth in SEQ ID NO:22. In another embodiment, the peptide sequence consists of an amino acid sequence set forth in SEQ ID NO:23. In other embodiments, the peptide sequence consists of an amino acid sequence set forth in SEQ ID NO:24. In one embodiment, the peptide sequence consists of an amino acid sequence set forth in SEQ ID NO:25. In another embodiment, the peptide sequence consists of an amino acid sequence set forth in SEQ ID NO:26. In other embodiments, the peptide sequence consists of an amino acid sequence set forth in SEQ ID NO:27. In one embodiment, the peptide sequence consists of an amino acid sequence set forth in SEQ ID NO:28. In another embodiment, the peptide sequence consists of an amino acid sequence set forth in SEQ ID NO:29. In other embodiments, the peptide sequence consists of an amino acid sequence set forth in SEQ ID NO:30. In one embodiment, the peptide sequence consists of an amino acid sequence set forth in SEQ ID NO:31. In another embodiment, the peptide sequence consists of an amino acid sequence set forth in SEQ ID NO:32. In other embodiments, the peptide sequence consists of an amino acid sequence set forth in SEQ ID NO:33. In one embodiment, the peptide sequence consists of an amino acid sequence set forth in SEQ ID NO:34. In another embodiment, the peptide sequence consists of an amino acid sequence set forth in SEQ ID NO:35. In other embodiments, the peptide sequence consists of an amino acid sequence set forth in SEQ ID NO:36. In one embodiment, the peptide sequence consists of an amino acid sequence set forth in SEQ ID NO:37. In another embodiment, the peptide sequence consists of an amino acid sequence set forth in SEQ ID NO:38. In other embodiments, the peptide sequence consists of an amino acid sequence set forth in SEQ ID NO:39. In one embodiment, the peptide sequence consists of an amino acid sequence set forth in SEQ ID NO:40. In another embodiment, the peptide sequence consists of an amino acid sequence set forth in SEQ ID NO:41. In other embodiments, the peptide sequence consists of an amino acid sequence set forth in SEQ ID NO:42. In one embodiment, the peptide sequence consists of an amino acid sequence set forth in SEQ ID NO:43. In another embodiment, the peptide sequence consists of an amino acid sequence set forth in SEQ ID NO:44. In other embodiments, the peptide sequence consists of an amino acid sequence set forth in SEQ ID NO:45. In one embodiment, the peptide sequence consists of an amino acid sequence set forth in SEQ ID NO:46. In another embodiment, the peptide sequence consists of an amino acid sequence set forth in SEQ ID NO:47. In other embodiments, the peptide sequence consists of an amino acid sequence set forth in SEQ ID NO:48. In one embodiment, the peptide sequence consists of an amino acid sequence set forth in SEQ ID NO:49. In another embodiment, the peptide sequence consists of an amino acid sequence set forth in SEQ ID NO:50. In other embodiments, the peptide sequence consists of an amino acid sequence set forth in SEQ ID NO: 51. In one embodiment, the peptide sequence consists of an amino acid sequence set forth in SEQ ID NO:52. In another embodiment, the peptide sequence consists of an amino acid sequence set forth in SEQ ID NO:53. In other embodiments, the peptide sequence consists of an amino acid sequence set forth in SEQ ID NO:54. In one embodiment, the peptide sequence consists of an amino acid sequence set forth in SEQ ID NO:55. In another embodiment, the peptide sequence consists of an amino acid sequence set forth in SEQ ID NO:56. In other embodiments, the peptide sequence consists of an amino acid sequence set forth in SEQ ID NO:57. In one embodiment, the peptide sequence consists of an amino acid sequence set forth in SEQ ID NO:58. In another embodiment, the peptide sequence consists of an amino acid sequence set forth in SEQ ID NO:59. In other embodiments, the peptide sequence consists of an amino acid sequence set forth in SEQ ID NO:60. In one embodiment, the peptide sequence consists of an amino acid sequence set forth in SEQ ID NO:61. In another embodiment, the peptide sequence consists of an amino acid sequence set forth in SEQ ID NO:62. In other embodiments, the peptide sequence consists of an amino acid sequence set forth in SEQ ID NO:63. In one embodiment, the peptide sequence consists of an amino acid sequence set forth in SEQ ID NO:64. In another embodiment, the peptide sequence consists of an amino acid sequence set forth in SEQ ID NO:65. In other embodiments, the peptide sequence consists of an amino acid sequence set forth in SEQ ID NO:66. In one embodiment, the peptide sequence consists of an amino acid sequence set forth in SEQ ID NO:67. In another embodiment, the peptide sequence consists of an amino acid sequence set forth in SEQ ID NO:68. In other embodiments, the peptide sequence consists of an amino acid sequence set forth in SEQ ID NO:69. In one embodiment, the peptide sequence consists of an amino acid sequence set forth in SEQ ID NO:70. In another embodiment, the peptide sequence consists of an amino acid sequence set forth in SEQ ID NO:71. In other embodiments, the peptide sequence consists of an amino acid sequence set forth in SEQ ID NO:72. In one embodiment, the peptide sequence consists of an amino acid sequence set forth in SEQ ID NO:73. In another embodiment, the peptide sequence consists of an amino acid sequence set forth in SEQ ID NO:74. In other embodiments, the peptide sequence consists of an amino acid sequence set forth in SEQ ID NO:75. In one embodiment, the peptide sequence consists of an amino acid sequence set forth in SEQ ID NO:76. In another embodiment, the peptide sequence consists of an amino acid sequence set forth in SEQ ID NO:77. In other embodiments, the peptide sequence consists of an amino acid sequence set forth in SEQ ID NO:78. In one embodiment, the peptide sequence consists of an amino acid sequence set forth in SEQ ID NO:79. In another embodiment, the peptide sequence consists of an amino acid sequence set forth in SEQ ID NO:80. In other embodiments, the peptide sequence consists of an amino acid sequence set forth in SEQ ID NO:81. In one embodiment, the peptide sequence consists of an amino acid sequence set forth in SEQ ID NO:82. In another embodiment, the peptide sequence consists of an amino acid sequence set forth in SEQ ID NO:83. In other embodiments, the peptide sequence consists of an amino acid sequence set forth in SEQ ID NO:84. In one embodiment, the peptide sequence consists of an amino acid sequence set forth in SEQ ID NO:85. In another embodiment, the peptide sequence consists of an amino acid sequence set forth in SEQ ID NO:86. In other embodiments, the peptide sequence consists of an amino acid sequence set forth in SEQ ID NO: 87. In one embodiment, the peptide sequence consists of an amino acid sequence set forth in SEQ ID NO:88. In another embodiment, the peptide sequence consists of an amino acid sequence set forth in SEQ ID NO:89. In other embodiments, the peptide sequence consists of an amino acid sequence set forth in SEQ ID NO:90. In one embodiment, the peptide sequence consists of an amino acid sequence set forth in SEQ ID NO:91. In another embodiment, the peptide sequence consists of an amino acid sequence set forth in SEQ ID NO:92. In other embodiments, the peptide sequence consists of an amino acid sequence set forth in SEQ ID NO:93. In one embodiment, the peptide sequence consists of an amino acid sequence set forth in SEQ ID NO:94. In another embodiment, the peptide sequence consists of an amino acid sequence set forth in SEQ ID NO:95. In other embodiments, the peptide sequence consists of an amino acid sequence set forth in SEQ ID NO:96. In one embodiment, the peptide sequence consists of an amino acid sequence set forth in SEQ ID NO:97. In another embodiment, the peptide sequence consists of an amino acid sequence set forth in SEQ ID NO:98. In other embodiments, the peptide sequence consists of an amino acid sequence set forth in SEQ ID NO:138. In one embodiment, the peptide sequence consists of an amino acid sequence set forth in SEQ ID NO:139. In another embodiment, the peptide sequence consists of an amino acid sequence set forth in SEQ ID NO:140. In other embodiments, the peptide sequence consists of an amino acid sequence set forth in SEQ ID NO:141. In one embodiment, the peptide sequence consists of an amino acid sequence set forth in SEQ ID NO:142. In another embodiment, the peptide sequence consists of an amino acid sequence set forth in SEQ ID NO:143. In other embodiments, the peptide sequence consists of an amino acid sequence set forth in SEQ ID NO:144. In one embodiment, the peptide sequence consists of an amino acid sequence set forth in SEQ ID NO:145. In another embodiment, the peptide sequence consists of an amino acid sequence set forth in SEQ ID NO:146. In other embodiments, the peptide sequence consists of an amino acid sequence set forth in SEQ ID NO:147. In one embodiment, the peptide sequence consists of an amino acid sequence set forth in SEQ ID NO:148. In another embodiment, the peptide sequence consists of an amino acid sequence set forth in SEQ ID NO:149. In other embodiments, the peptide sequence consists of an amino acid sequence set forth in SEQ ID NO:150. In one embodiment, the peptide sequence consists of an amino acid sequence set forth in SEQ ID NO:151. In another embodiment, the peptide sequence consists of an amino acid sequence set forth in SEQ ID NO:152. In other embodiments, the peptide sequence consists of an amino acid sequence set forth in SEQ ID NO:153. In one embodiment, the peptide sequence consists of an amino acid sequence set forth in SEQ ID NO:154. In another embodiment, the peptide sequence consists of an amino acid sequence set forth in SEQ ID NO:155. In other embodiments, the peptide sequence consists of an amino acid sequence set forth in SEQ ID NO:156. In one embodiment, the peptide sequence consists of an amino acid sequence set forth in SEQ ID NO:157. In another embodiment, the peptide sequence consists of an amino acid sequence set forth in SEQ ID NO:158. In other embodiments, the peptide sequence consists of an amino acid sequence set forth in SEQ ID NO:159. In one embodiment, the peptide sequence consists of an amino acid sequence set forth in SEQ ID NO:160. In another embodiment, the peptide sequence consists of an amino acid sequence set forth in SEQ ID NO:161. In other embodiments, the peptide sequence consists of an amino acid sequence set forth in SEQ ID NO:162. In one embodiment, the peptide sequence consists of an amino acid sequence set forth in SEQ ID NO:163. In another embodiment, the peptide sequence consists of an amino acid sequence set forth in SEQ ID NO:164. In other embodiments, the peptide sequence consists of an amino acid sequence set forth in SEQ ID NO:165. In one embodiment, the peptide sequence consists of an amino acid sequence set forth in SEQ ID NO:166. In another embodiment, the peptide sequence consists of an amino acid sequence set forth in SEQ ID NO:167. In other embodiments, the peptide sequence consists of an amino acid sequence set forth in SEQ ID NO:168. In another embodiment, the peptide sequence consists of an amino acid sequence set forth in SEQ ID NO:192. In other embodiments, the peptide sequence consists of an amino acid sequence set forth in SEQ ID NO:193. In one embodiment, the peptide sequence consists of an amino acid sequence set forth in SEQ ID NO:194. In another embodiment, the peptide sequence consists of an amino acid sequence set forth in SEQ ID NO:195. In other embodiments, the peptide sequence consists of an amino acid sequence set forth in SEQ ID NO:196. In one embodiment, the peptide sequence consists of an amino acid sequence set forth in SEQ ID NO:197. In another embodiment, the peptide sequence consists of an amino acid sequence set forth in SEQ ID NO:198. In other embodiments, the peptide sequence consists of an amino acid sequence set forth in SEQ ID NO:199. In one embodiment, the peptide sequence consists of an amino acid sequence set forth in SEQ ID NO:200. In another embodiment, the peptide sequence consists of an amino acid sequence set forth in SEQ ID NO:201. In other embodiments, the peptide sequence consists of an amino acid sequence set forth in SEQ ID NO:202. In one embodiment, the peptide sequence consists of an amino acid sequence set forth in SEQ ID NO:203. In another embodiment, the peptide sequence consists of an amino acid sequence set forth in SEQ ID NO:204. In certain embodiments of the various peptide sequences provided herein, the R residue at the N-terminus is deleted.

### Nucleic Acid Molecules

Also provided are nucleic acid molecules encoding peptide sequences provided herein, including subsequences, sequence variants and modified forms of the sequences listed in the Sequence Listing or Table 1, and in PCT Pub. No. WO 2013/006486 and US Pub. No. 2013/0023474, as well as PCT Publ. No. WO 2014/085365, and vectors that include nucleic acid encoding the peptides used in the methods described herein. Accordingly, “nucleic acids” include those that encode the exemplified peptide sequences disclosed herein, as well as those encoding functional subsequences, sequence variants and modified forms of the exemplified peptide sequences, so long as the foregoing retain at least detectable or measureable activity or function useful in the treatment or prevention of a gastrointestinal motility-related disorder, such as constipation, or in stimulating bowel function.

Nucleic acid, which can also be referred to herein as a gene, polynucleotide, nucleotide sequence, primer, oligonucleotide or probe, refers to natural or modified purine- and pyrimidine-containing polymers of any length, either polyribonucleotides or polydeoxyribonucleotides or mixed polyribo-polydeoxyribo nucleotides and α-anomeric forms thereof. The two or more purine- and pyrimidine-containing polymers are typically linked by a phosphoester bond or analog thereof. The terms can be used interchangeably to refer to all forms of nucleic acid, including deoxyribonucleic acid (DNA) and ribonucleic acid (RNA). The nucleic acids can be single strand, double, or triplex, linear or circular. Nucleic acids include genomic DNA and cDNA. RNA nucleic acid can be spliced or unspliced mRNA, rRNA, tRNA or antisense. Nucleic acids include naturally occurring, synthetic, as well as nucleotide analogs and derivatives.

The terms “SNP” or “single nucleotide polymorphism” refer to a variation in a single nucleotide that occurs at a specific position in the genome. Single-nucleotide polymorphisms may fall within coding sequences of genes, non-coding regions of genes, or in the intergenic regions (regions between genes). SNPs within a coding sequence do not necessarily change the amino acid sequence of the protein that is produced, due to degeneracy of the genetic code.

As a result of the degeneracy of the genetic code, the nucleic acid molecules provided herein include sequences degenerate with respect to nucleic acid molecules encoding the peptide sequences useful in the methods provided herein. Thus, degenerate nucleic acid sequences encoding peptide sequences, including subsequences, variants and modified forms of the peptide sequences exemplified herein (e.g., in the Sequence Listing or Table 1), are provided. The term “complementary,” when used in reference to a nucleic acid sequence, means the referenced regions are 100% complementary, i.e., exhibit 100% base pairing with no mismatches.

Nucleic acid can be produced using any of a variety of known standard cloning and chemical synthesis methods, and can be altered intentionally by site-directed mutagenesis or other recombinant techniques known to one skilled in the art. Purity of polynucleotides can be determined through, for example, sequencing, gel electrophoresis, and UV spectrometry.

Nucleic acids may be inserted into a nucleic acid construct in which expression of the nucleic acid is influenced or regulated by an “expression control element,” referred to herein as an “expression cassette.” The term “expression control element” refers to one or more nucleic acid sequence elements that regulate or influence expression of a nucleic acid sequence to which it is operatively linked. An expression control element can include, as appropriate, promoters, enhancers, transcription terminators, gene silencers, a start codon (e.g., ATG) in front of a protein-encoding gene, etc.

An expression control element operatively linked to a nucleic acid sequence controls transcription and, as appropriate, translation of the nucleic acid sequence. The term “operatively linked” refers to a juxtaposition wherein the referenced components are in a relationship permitting them to function in their intended manner. Typically, expression control elements are juxtaposed at the 5′ or the 3′ ends of the genes but can also be intronic.

Expression control elements include elements that activate transcription constitutively, that are inducible (i.e., require an external signal or stimuli for activation), or derepressible (i.e., require a signal to turn transcription off; when the signal is no longer present, transcription is activated or “derepressed”). Also included in the expression cassettes provided herein are control elements sufficient to render gene expression controllable for specific cell types or tissues (i.e., tissue-specific control elements). Typically, such elements are located upstream or downstream (i.e., 5′ or 3′) of the coding sequence. Promoters are generally positioned 5′ of the coding sequence. Promoters, produced by recombinant DNA or synthetic techniques, can be used to provide for transcription of the polynucleotides provided herein. A “promoter” typically means a minimal sequence element sufficient to direct transcription.

Nucleic acids may be inserted into a plasmid for transformation into a host cell and for subsequent expression and/or genetic manipulation. A plasmid is a nucleic acid that can be stably propagated in a host cell; plasmids may optionally contain expression control elements in order to drive expression of the nucleic acid. For purposes of this invention, a vector is synonymous with a plasmid. Plasmids and vectors generally contain at least an origin of replication for propagation in a cell and a promoter. Plasmids and vectors may also include an expression control element for expression in a host cell, and are therefore useful for expression and/or genetic manipulation of nucleic acids encoding peptide sequences, expressing peptide sequences in host cells and organisms, or producing peptide sequences, for example.

As used herein, the term “transgene” means a polynucleotide that has been introduced into a cell or organism by artifice. For example, in a cell having a transgene, the transgene has been introduced by genetic manipulation or “transformation” of the cell. A cell or progeny thereof into which the transgene has been introduced is referred to as a “transformed cell” or “transformant.” Typically, the transgene is included in progeny of the transformant or becomes a part of the organism that develops from the cell. Transgenes may be inserted into the chromosomal DNA or maintained as a self-replicating plasmid, YAC, minichromosome, or the like.

Bacterial system promoters include T7 and inducible promoters such as pL of bacteriophage λ, plac, ptrp, ptac (ptrp-lac hybrid promoter) and tetracycline-responsive promoters. Insect cell system promoters include constitutive or inducible promoters (e.g., ecdysone). Mammalian cell constitutive promoters include SV40, RSV, bovine papilloma virus (BPV) and other virus promoters, or inducible promoters derived from the genome of mammalian cells (e.g., metallothionein IIA promoter; heat shock promoter) or from mammalian viruses (e.g., the adenovirus late promoter; the inducible mouse mammary tumor virus long terminal repeat). Alternatively, a retroviral genome can be genetically modified for introducing and directing expression of a peptide sequence in appropriate host cells.

As methods and uses provided herein include in vivo delivery, expression systems further include vectors designed for in vivo use. Particular non-limiting examples include adenoviral vectors (U.S. Pat. Nos. 5,700,470 and 5,731,172), adeno-associated vectors (U.S. Pat. No. 5,604,090), herpes simplex virus vectors (U.S. Pat. No. 5,501,979), retroviral vectors (U.S. Pat. Nos. 5,624,820, 5,693,508 and 5,674,703), BPV vectors (U.S. Pat. No. 5,719,054), CMV vectors (U.S. Pat. No. 5,561,063) and parvovirus, rotavirus, Norwalk virus and lentiviral vectors (see, e.g., U.S. Pat. No. 6,013,516). Vectors include those that deliver genes to cells of the intestinal tract, including the stem cells (Croyle et al., Gene Ther. 5:645 (1998); S. J. Henning, Adv. Drug Deliv. Rev. 17:341 (1997), U.S. Pat. Nos. 5,821,235 and 6,110,456). Many of these vectors have been approved for human studies.

Yeast vectors include constitutive and inducible promoters (see, e.g., Ausubel et al., In: Current Protocols in Molecular Biology, Vol. 2, Ch. 13, ed., Greene Publish. Assoc. & Wiley Interscience, 1988; Grant et al. Methods in Enzymology, 153:516 (1987), eds. Wu & Grossman; Bitter Methods in Enzymology, 152:673 (1987), eds. Berger & Kimmel, Acad. Press, N.Y.; and, Strathern et al., The Molecular Biology of the Yeast Saccharomyces (1982) eds. Cold Spring Harbor Press, Vols. I and II). A constitutive yeast promoter such as ADH or LEU2 or an inducible promoter such as GAL may be used (R. Rothstein In: DNA Cloning, A Practical Approach, Vol. 11, Ch. 3, ed. D. M. Glover, IRL Press, Wash., D.C., 1986). Vectors that facilitate integration of foreign nucleic acid sequences into a yeast chromosome, via homologous recombination for example, are known in the art. Yeast artificial chromosomes (YAC) are typically used when the inserted polynucleotides are too large for more conventional vectors (e.g., greater than about 12 Kb).

Expression vectors also can contain a selectable marker conferring resistance to a selective pressure or identifiable marker (e.g., beta-galactosidase), thereby allowing cells having the vector to be selected for, grown and expanded. Alternatively, a selectable marker can be on a second vector that is co-transfected into a host cell with a first vector containing a nucleic acid encoding a peptide sequence. Selection systems include, but are not limited to, herpes simplex virus thymidine kinase gene (Wigler et al., Cell 11:223 (1977)), hypoxanthine-guanine phosphoribosyltransferase gene (Szybalska et al., Proc. Natl. Acad. Sci. USA 48:2026 (1962)), and adenine phosphoribosyltransferase (Lowy et al., Cell 22:817 (1980)) genes that can be employed in tk-, hgprt- or aprt-cells, respectively. Additionally, antimetabolite resistance can be used as the basis of selection for dhfr, which confers resistance to methotrexate (O'Hare et al., Proc. Natl. Acad. Sci. USA 78:1527 (1981)); the gpt gene, which confers resistance to mycophenolic acid (Mulligan et al., Proc. Natl. Acad. Sci. USA 78:2072 (1981)); neomycin gene, which confers resistance to aminoglycoside G-418 (Colberre-Garapin et al, J. Mol. Biol. 150:1(1981)); puromycin; and hygromycin gene, which confers resistance to hygromycin (Santerre et al., Gene 30:147 (1984)). Additional selectable genes include trpB, which allows cells to utilize indole in place of tryptophan; hisD, which allows cells to utilize histinol in place of histidine (Hartman et al., Proc. Natl. Acad. Sci. USA 85:8047 (1988)); and ODC (ornithine decarboxylase), which confers resistance to the ornithine decarboxylase inhibitor, 2-(difluoromethyl)-DL-ornithine, DFMO (McConlogue (1987) In: Current Communications in Molecular Biology, Cold Spring Harbor Laboratory).

### Cell Lines and Animal Models

In certain embodiments, also provided is a transformed cell(s) (in vitro, ex vivo and in vivo) and host cells that produce a variant or fusion of FGF19 and/or FGF21 as set forth herein, where expression of the variant or fusion of FGF19 and/or FGF21 is conferred by a nucleic acid encoding the variant or fusion of FGF19 and/or FGF21. As used herein, a “transformed” or “host” cell is a cell into which a nucleic acid is introduced that can be propagated and/or transcribed for expression of an encoded peptide sequence. The term also includes any progeny or subclones of the host cell. Transformed and host cells that express peptide sequences provided herein typically include a nucleic acid that encodes the peptide sequence. In one embodiment, a transformed or host cell is a prokaryotic cell. In another embodiment, a transformed or host cell is a eukaryotic cell. In various aspects, the eukaryotic cell is a yeast or mammalian (e.g., human, primate, etc.) cell.

Transformed and host cells include but are not limited to microorganisms such as bacteria and yeast; and plant, insect and mammalian cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for transient or stable propagation or expression.

For gene therapy uses and methods, a transformed cell can be in a subject. A cell in a subject can be transformed with a nucleic acid that encodes a peptide sequence as set forth herein in vivo. Alternatively, a cell can be transformed in vitro with a transgene or polynucleotide, and then transplanted into a tissue of subject in order to effect treatment. Alternatively, a primary cell isolate or an established cell line can be transformed with a transgene or polynucleotide that encodes a variant of FGF19 and/or FGF21 or a fusion/chimeric sequence (or variant) thereof, such as a chimeric peptide sequence including all or a portion of FGF19, or including all or a portion of FGF21, and then optionally transplanted into a tissue of a subject.

Non-limiting target cells for expression of peptide sequences, particularly for expression in vivo, include pancreas cells (islet cells), muscle cells, mucosal cells and endocrine cells. Such endocrine cells can provide inducible production (secretion) of a variant of FGF19 and/or FGF21, or a fusion/chimeric sequence (or variant) thereof, such as a chimeric peptide sequence including all or a portion of FGF19, or including all or a portion of FGF21. Additional cells to transform include stem cells or other multipotent or pluripotent cells, for example, progenitor cells that differentiate into the various pancreas cells (islet cells), muscle cells, mucosal cells and endocrine cells. Targeting stem cells provides longer term expression of peptide sequences provided herein.

As used herein, the term “cultured,” when used in reference to a cell, means that the cell is grown in vitro. A particular example of such a cell is a cell isolated from a subject, and grown or adapted for growth in tissue culture. Another example is a cell genetically manipulated in vitro, and transplanted back into the same or a different subject.

The term “isolated,” when used in reference to a cell, means a cell that is separated from its naturally occurring in vivo environment. “Cultured” and “isolated” cells may be manipulated by the hand of man, such as genetically transformed. These terms include any progeny of the cells, including progeny cells that may not be identical to the parental cell due to mutations that occur during cell division. The terms do not include an entire human being.

Nucleic acids encoding peptide sequences provided herein can be introduced for stable expression into cells of a whole organism. Such organisms, including non-human transgenic animals, are useful for studying the effect of peptide expression in a whole animal and therapeutic benefit. For example, as disclosed herein, production of a variant of FGF19 and/or FGF21 or a fusion/chimeric sequence (or variant) thereof, such as a chimeric peptide sequence including all or a portion of FGF19, or including all or a portion of FGF21 as set forth herein, in mice.

Mice strains that develop or are susceptible to developing a particular disease (e.g., diabetes, degenerative disorders, cancer, etc.) are also useful for introducing therapeutic proteins as described herein in order to study the effect of therapeutic protein expression in the disease-susceptible mouse. Transgenic and genetic animal models that are susceptible to particular disease or physiological conditions, such as streptozotocin (STZ)-induced diabetic (STZ) mice, are appropriate targets for expressing variants of FGF19 and/or FGF21, fusions/chimeric sequences (or variant) thereof, such as a chimeric peptide sequence including all or a portion of FGF19, or including all or a portion of FGF21, as set forth herein. Thus, in certain embodiments, there are provided non-human transgenic animals that produce a variant of FGF19 and/or FGF21, or a fusion/chimeric sequence (or variant) thereof, such as a chimeric peptide sequence including all or a portion of FGF19, or including all or a portion of FGF21, the production of which is not naturally occurring in the animal which is conferred by a transgene present in somatic or germ cells of the animal.

The term “transgenic animal” refers to an animal whose somatic or germ line cells bear genetic information received, directly or indirectly, by deliberate genetic manipulation at the subcellular level, such as by microinjection or infection with recombinant virus. The term “transgenic” further includes cells or tissues (i.e., “transgenic cell,” “transgenic tissue”) obtained from a transgenic animal genetically manipulated as described herein. In the present context, a “transgenic animal” does not encompass animals produced by classical crossbreeding or in vitro fertilization, but rather denotes animals in which one or more cells receive a nucleic acid molecule. Transgenic animals provided herein can be either heterozygous or homozygous with respect to the transgene. Methods for producing transgenic animals, including mice, sheep, pigs and frogs, are well known in the art (see, e.g., U.S. Pat. Nos. 5,721,367, 5,695,977, 5,650,298, and 5,614,396) and, as such, are additionally included.

Peptide sequences, nucleic acids encoding peptide sequences, vectors and transformed host cells expressing peptide sequences include isolated and purified forms. The term “isolated,” when used as a modifier of a composition provided herein, means that the composition is separated, substantially, completely, or at least in part, from one or more components in an environment. Generally, compositions that exist in nature, when isolated, are substantially free of one or more materials with which they normally associate with in nature, for example, one or more protein, nucleic acid, lipid, carbohydrate or cell membrane. The term “isolated” does not exclude alternative physical forms of the composition, such as variants, modifications or derivatized forms, fusions and chimeras, multimers/oligomers, etc., or forms expressed in host cells. The term “isolated” also does not exclude forms (e.g., pharmaceutical compositions, combination compositions, etc.) in which there are combinations therein, any one of which is produced by the hand of man. An “isolated” composition can also be “purified” when free of some, a substantial number of, or most or all of one or more other materials, such as a contaminant or an undesired substance or material.

As used herein, the term “recombinant,” when used as a modifier of peptide sequences, nucleic acids encoding peptide sequences, etc., means that the compositions have been manipulated (i.e., engineered) in a fashion that generally does not occur in nature (e.g., in vitro). A particular example of a recombinant peptide would be where a peptide sequence provided herein is expressed by a cell transfected with a nucleic acid encoding the peptide sequence. A particular example of a recombinant nucleic acid would be a nucleic acid (e.g., genomic or cDNA) encoding a peptide sequence cloned into a plasmid, with or without 5′, 3′ or intron regions that the gene is normally contiguous within the genome of the organism. Another example of a recombinant peptide or nucleic acid is a hybrid or fusion sequence, such as a chimeric peptide sequence comprising a portion of FGF19 and a portion of FGF21.

### Particular Modifications to Enhance Peptide Function

It is frequently beneficial, and sometimes imperative, to improve one of more physical properties of the treatment modalities disclosed herein and/or the manner in which they are administered. Improvements of physical properties include, for example, modulating immunogenicity; methods of increasing solubility, bioavailability, serum half-life, and/or therapeutic half-life; and/or modulating biological activity. Certain modifications may also be useful to, for example, raise of antibodies for use in detection assays (e.g., epitope tags) and to provide for ease of protein purification. Such improvements must generally be imparted without adversely impacting the bioactivity of the treatment modality and/or increasing its immunogenicity.

Pegylation of is one particular modification contemplated herein, while other modifications include, but are not limited to, glycosylation (N- and O-linked); polysialylation; albumin fusion molecules comprising serum albumin (e.g., human serum albumin (HSA), cyno serum albumin, or bovine serum albumin (BSA)); albumin binding through, for example a conjugated fatty acid chain (acylation); and Fc-fusion proteins.

Pegylation:

The clinical effectiveness of protein therapeutics is often limited by short plasma half-life and susceptibility to protease degradation. Studies of various therapeutic proteins (e.g., filgrastim) have shown that such difficulties may be overcome by, for example, conjugating or linking the protein to any of a variety of nonproteinaceous polymers, e.g., polyethylene glycol (PEG), polypropylene glycol, or polyoxyalkylenes. This is frequently effected by a linking moiety covalently bound to both the protein and the nonproteinaceous polymer, e.g., a PEG. Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity. In addition to the beneficial effects of pegylation on pharmacokinetic parameters, pegylation itself may enhance activity.

PEGS suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGS are contemplated by the present disclosure. A molecular weight of the PEG used in embodiments provided herein is not restricted to any particular range, and examples are set forth elsewhere herein; by way of example, certain embodiments have molecular weights between 5 kDa and 20 kDa, while other embodiments have molecular weights between 4 kDa and 10 kDa.

In other embodiments, provided herein are compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. Cation exchange chromatography may be used to separate conjugates, and a fraction is then identified which contains the conjugate having, for example, the desired number of PEGS attached, purified free from unmodified protein sequences and from conjugates having other numbers of PEGS attached.

Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry.

General pegylation strategies known in the art can be applied herein. PEG may be bound to a polypeptide provided herein via a terminal reactive group (a “spacer” or “linker”) which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxysuccinylimide. Another activated polyethylene glycol which may be bound to a free amino group is 2,4-bis(O-methoxypolyethyleneglycol)-6-chloro-s-triazine, which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences provided herein to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH≥7), and longer reaction time tend to increase the number of PEGs attached. Various means known in the art may be used to terminate the reaction. In some embodiments, the reaction is terminated by acidifying the reaction mixture and freezing at, e.g., −20° C. Pegylation of various molecules is discussed in, for example, U.S. Pat. Nos. 5,252,714; 5,643,575; 5,919,455; 5,932,462; and 5,985,263.

In some embodiments, also provided herein are uses of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., XTEN technology; Amunix; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

Glycosylation:

As used herein, “glycosylation” is meant to broadly refer to the enzymatic process by which glycans are attached to proteins, lipids or other organic molecules. The use of the term “glycosylation” herein is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present.

Glycosylation can dramatically affect the physical properties (e.g., solubility) of polypeptides and can also be important in protein stability, secretion, and subcellular localization. Glycosylated polypeptides may also exhibit enhanced stability or may improve one or more pharmacokinetic properties, such as half-life. In addition, solubility improvements can, for example, enable the generation of formulations more suitable for pharmaceutical administration than formulations comprising the non-glycosylated polypeptide.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment comprises the generation and use of N-glycosylation variants.

The polypeptide sequences provided herein may optionally be altered through changes at the nucleic acid level, particularly by mutating the nucleic acid encoding the polypeptide at preselected bases such that codons are generated that will translate into the desired amino acids. Various cell lines can be used to produce proteins that are glycosylated. One non-limiting example is Dihydrofolate reductase (DHFR)-deficient Chinese Hamster Ovary (CHO) cells, which are a commonly used host cell for the production of recombinant glycoproteins. These cells do not express the enzyme beta-galactoside alpha-2,6-sialyltransferase and therefore do not add sialic acid in the alpha-2,6 linkage to N-linked oligosaccharides of glycoproteins produced in these cells.

Polysialylation:

In certain embodiments, also provided herein is the use of polysialylation, the conjugation of polypeptides to the naturally occurring, biodegradable α-(2→8) linked polysialic acid (“PSA”) in order to improve the polypeptides' stability and in vivo pharmacokinetics.

Albumin Fusion:

Additional suitable components and molecules for conjugation include albumins such as human serum albumin (HSA), cyno serum albumin, and bovine serum albumin (BSA).

In some embodiments, albumin is conjugated to a drug molecule (e.g., a polypeptide described herein) at the carboxyl terminus, the amino terminus, both the carboxyl and amino termini, and internally (see, e.g., U.S. Pat. Nos. 5,876,969 and 7,056,701).

In the HSA-drug molecule conjugates embodiments provided herein, various forms of albumin may be used, such as albumin secretion pre-sequences and variants thereof, fragments and variants thereof, and HSA variants. Such forms generally possess one or more desired albumin activities. In additional embodiments, fusion proteins are provided herein comprising a polypeptide drug molecule fused directly or indirectly to albumin, an albumin fragment, an albumin variant, etc., wherein the fusion protein has a higher plasma stability than the unfused drug molecule and/or the fusion protein retains the therapeutic activity of the unfused drug molecule. In some embodiments, the indirect fusion is effected by a linker, such as a peptide linker or modified version thereof.

As alluded to above, fusion of albumin to one or more polypeptides provided herein can, for example, be achieved by genetic manipulation, such that the nucleic acid coding for HSA, or a fragment thereof, is joined to the nucleic acid coding for the one or more polypeptide sequences.

Alternative Albumin Binding Strategies:

Several albumin—binding strategies have been developed as alternatives to direct fusion and may be used with the agents described herein. By way of example, in certain embodiments, provided herein is albumin binding through a conjugated fatty acid chain (acylation) and fusion proteins which comprise an albumin binding domain (ABD) polypeptide sequence and the sequence of one or more of the polypeptides described herein.

Fusion of albumin to a peptide sequence can, for example, be achieved by genetic manipulation, such that the DNA coding for HSA (human serum albumin), or a fragment thereof, is joined to the DNA coding for a peptide sequence. Thereafter, a suitable host can be transformed or transfected with the fused nucleotide sequence in the form of, for example, a suitable plasmid, so as to express a fusion polypeptide. The expression may be effected in vitro from, for example, prokaryotic or eukaryotic cells, or in vivo from, for example, a transgenic organism. In some embodiments, the expression of the fusion protein is performed in mammalian cell lines, for example, CHO cell lines.

Further means for genetically fusing target proteins or peptides to albumin include a technology known as Albufuse® (Novozymes Biopharma A/S; Denmark), and the conjugated therapeutic peptide sequences frequently become much more effective with better uptake in the body. The technology has been utilized commercially to produce Albuferon® (Human Genome Sciences), a combination of albumin and interferon α-2B used to treat hepatitis C infection.

Another embodiment entails the use of one or more human domain antibodies (dAb). dAbs are the smallest functional binding units of human antibodies (IgGs) and have favorable stability and solubility characteristics. The technology entails a dAb(s) conjugated to HSA (thereby forming a “AlbudAb”; see, e.g., EP1517921B, WO2005/118642 and WO2006/051288) and a molecule of interest (e.g., a peptide sequence provided herein). AlbudAbs are often smaller and easier to manufacture in microbial expression systems, such as bacteria or yeast, than current technologies used for extending the serum half-life of peptides. As HSA has a half-life of about three weeks, the resulting conjugated molecule improves the half-life. Use of the dAb technology may also enhance the efficacy of the molecule of interest.

Conjugation with Other Molecules:

Additional suitable components and molecules for conjugation include, for example, thyroglobulin; tetanus toxoid; Diphtheria toxoid; polyamino acids such as poly(D-lysine:D-glutamic acid); VP6 polypeptides of rotaviruses; influenza virus hemagglutinin, influenza virus nucleoprotein; Keyhole Limpet Hemocyanin (KLH); and hepatitis B virus core protein and surface antigen; or any combination of the foregoing.

Thus, in certain embodiments, conjugation of one or more additional components or molecules at the N- and/or C-terminus of a polypeptide sequence, such as another polypeptide (e.g., a polypeptide having an amino acid sequence heterologous to the subject polypeptide), or a carrier molecule is also contemplated. Thus, an exemplary polypeptide sequence can be provided as a conjugate with another component or molecule.

A polypeptide may also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria; and dendritic cells. Such conjugated forms, if desired, can be used to produce antibodies against a polypeptide provided herein.

Fc-Fusion Molecules:

In certain embodiments, the amino- or carboxyl-terminus of a polypeptide sequence provided herein is fused with an immunoglobulin Fc region to form a fusion conjugate (or fusion molecule). In a specific embodiment, the immunoglobulin Fc region is a human Fc region. Fusion conjugates have been shown to increase the systemic half-life of biopharmaceuticals, and thus the biopharmaceutical product may require less frequent administration. In certain embodiments, the half-life is increased as compared to the same polypeptide that is not fused to an immunoglobulin Fc region.

Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

Well-known and validated Fc-fusion drugs consist of two copies of a biopharmaceutical linked to the Fc region of an antibody to improve pharmacokinetics, solubility, and production efficiency. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

In some embodiments, provided herein is a fusion of M70 to a human antibody Fc fragment. In some embodiments, provided herein is a fusion of M69 to a human antibody Fc fragment. Such fusions can be useful in the treatment of bile acid related disorders and other metabolic disorders provided herein. In some embodiments, the Fc-fusion of M70 has a longer half-life. In specific embodiments, the longer half-life of the Fc-fusion of M70 is as compared to M70 that is not an Fc-fusion. In some embodiments, the Fc-fusion of M69 has a longer half-life. In specific embodiments, the longer half life of the Fc-fusion of M69 is as compared to M69 that is not an Fc-fusion. Such a long half-life makes these fusions suitable for once weekly, or less frequent dosing.

In some embodiments, the Fc-fusion comprises a linker. Exemplary flexible linkers include glycine polymers (G)ₙ, glycine-serine polymers, glycine-alanine polymers, alanine-serine polymers, and other flexible linkers. In certain embodiments, the linker is (G)₄S. In some embodiments, the linker is ((G)₄S)ₙ, where n is an integer of at least one. In some embodiments, the linker is ((G)₄S)₂. Glycine and glycine-serine polymers are relatively unstructured, and therefore may serve as a neutral tether between components. In some embodiments, the glycine-serine polymer is (GS)ₙ, where n is an integer of at least one. In some embodiments, the glycine-serine polymer is GSGGSₙ (SEQ ID NO:129), where n is an integer of at least one. In some embodiments, the glycine-serine polymer is GGGSₙ (SEQ ID NO:130), where n is an integer of at least one. In certain embodiments, the linker comprises an additional G residue at the N′ terminus of SEQ ID NO:130. In one embodiment, the linker is GGSG (SEQ ID NO:131). In one embodiment, the linker is GGSGG (SEQ ID NO:132). In one embodiment, the linker is GSGSG (SEQ ID NO:133). In one embodiment, the linker is GSGGG (SEQ ID NO:134). In one embodiment, the linker is GGGSG (SEQ ID NO:189). In one embodiment, the linker is GSSSG (SEQ ID NO:135).

Purification:

Additional suitable components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

Purification methods such as cation exchange chromatography may be used to separate conjugates by charge difference, which effectively separates conjugates into their various molecular weights. For example, the cation exchange column can be loaded and then washed with ˜20 mM sodium acetate, pH ˜4, and then eluted with a linear (0 M to 0.5 M) NaCl gradient buffered at a pH from 3 to 5.5, such as at pH ˜4.5. The content of the fractions obtained by cation exchange chromatography may be identified by molecular weight using conventional methods, for example, mass spectroscopy, SDS-PAGE, or other known methods for separating molecular entities by molecular weight. A fraction is then identified which contains the conjugate having the desired number of PEGs attached, purified free from unmodified protein sequences and from conjugates having other numbers of PEGS attached.

### Other Modifications

In certain embodiments, also provided herein is the use of other modifications, currently known or developed in the future, to improve one or more properties. Examples include hesylation, various aspects of which are described in, for example, U.S. Patent Appln. Nos. 2007/0134197 and 2006/0258607, and fusion molecules comprising SUMO as a fusion tag (LifeSensors, Inc.; Malvern, Pa.).

In still other embodiments, a peptide sequence provided herein is linked to a chemical agent (e.g., an immunotoxin or chemotherapeutic agent), including, but are not limited to, a cytotoxic agent, including taxol, cytochalasin B, gramicidin D, mitomycin, etoposide, tenoposide, vincristine, vinblastine, colchicin, doxorubicin, daunorubicin, and analogs or homologs thereof. Other chemical agents include, for example, antimetabolites (e.g., methotrexate, 6-mercaptopurine, 6-thioguanine, cytarabine, 5-fluorouracil decarbazine); alkylating agents (e.g., mechlorethamine, carmustine and lomustine, cyclothosphamide, busulfan, dibromomannitol, streptozotocin, mitomycin C, and cisplatin); antibiotics (e.g., bleomycin); and anti-mitotic agents (e.g., vincristine and vinblastine). Cytotoxins can be conjugated to a peptide provided herein using linker technology known in the art and described herein.

Further suitable components and molecules for conjugation include those suitable for detection in an assay. Particular non-limiting examples include detectable labels, such as a radioisotope (e.g., ¹²⁵I; ³⁵S, ³²P; ³³P), an enzyme which generates a detectable product (e.g., luciferase, β-galactosidase, horse radish peroxidase and alkaline phosphatase), a fluorescent protein, a chromogenic protein, dye (e.g., fluorescein isothiocyanate); fluorescence emitting metals (e.g., ¹⁵²Eu); chemiluminescent compounds (e.g., luminol and acridinium salts); bioluminescent compounds (e.g., luciferin); and fluorescent proteins. Indirect labels include labeled or detectable antibodies that bind to a peptide sequence, where the antibody may be detected.

In certain embodiments, a peptide sequence provided herein is conjugated to a radioactive isotope to generate a cytotoxic radiopharmaceutical (radioimmunoconjugates) useful as a diagnostic or therapeutic agent. Examples of such radioactive isotopes include, but are not limited to, iodine¹³¹, indium¹¹¹, yttrium⁹⁰ and lutetium¹⁷⁷. Methods for preparing radioimmunoconjugates are known to the skilled artisan. Examples of radioimmunoconjugates that are commercially available include ibritumomab, tiuxetan, and tositumomab.

Linkers:

Linkers and their use have been described above. Any of the foregoing components and molecules used to modify the polypeptide sequences provided herein may optionally be conjugated via a linker. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Exemplary flexible linkers include glycine polymers (G)ₙ, glycine-serine polymers (for example, (GS)ₙ, GSGGSₙ (SEQ ID NO:129) and GGGSₙ (SEQ ID NO:130), where n is an integer of at least one), glycine-alanine polymers, alanine-serine polymers, and other flexible linkers. Glycine and glycine-serine polymers are relatively unstructured, and therefore may serve as a neutral tether between components. Exemplary flexible linkers include, but are not limited to GGSG (SEQ ID NO:131), GGSGG (SEQ ID NO:132), GSGSG (SEQ ID NO:133), GSGGG (SEQ ID NO:134), GGGSG (SEQ ID NO:189), and GSSSG (SEQ ID NO:135). In certain embodiments, the linker is (G)4S. In some embodiments, the linker is ((G)4S)ₙ), where n is an integer of at least one. In some embodiments, the linker is ((G)4S)₂). In some embodiments, the glycine-serine polymer is (GS)ₙ, where n is an integer of at least one. In some embodiments, the glycine-serine polymer is GSGGSₙ (SEQ ID NO:129), where n is an integer of at least one. In some embodiments, the glycine-serine polymer is GGGSₙ (SEQ ID NO:130), where n is an integer of at least one. In certain embodiments, the linker comprises an additional G residue at the N′ terminus of SEQ ID NO:130. In one embodiment, the linker is GGSG (SEQ ID NO:131). In one embodiment, the linker is GGSGG (SEQ ID NO:132). In one embodiment, the linker is GSGSG (SEQ ID NO:133). In one embodiment, the linker is GSGGG (SEQ ID NO:134). In one embodiment, the linker is GGGSG (SEQ ID NO:189). In one embodiment, the linker is GSSSG (SEQ ID NO:135).

### Gastrointestinal Motility-Related Disorders and the Treatment or Prevention Thereof

The peptide sequences set forth herein can be used in methods for treating or preventing a gastrointestinal motility-related disorder, such as constipation. The peptide sequences set forth herein can also be used in methods for stimulating bowel function. In various embodiments, the methods include administering a peptide sequence, such as a FGF19 or FGF21 variant, fusion or chimera disclosed herein (e.g., in the Sequence Listing or Table 1), or a subsequence, a variant or modified form of a FGF19 or FGF21 variant, fusion or chimera disclosed herein (e.g., the Sequence Listing or Table 1), to a subject in an amount effective for treating or preventing a gastrointestinal motility-related disorder, for treating or preventing constipation or for stimulating bowel function.

Provided herein are methods for treating or preventing a gastrointestinal motility-related disorder in a subject by administering a peptide sequence described herein to the subject. As used herein, the term “gastrointestinal motility-related disorder,” also known as “GI motility-related disorder,” “gastrointestinal motility disorder,” or “GI motility disorder,” refers to a condition that is characterized by abnormal motility or abnormal sensitivity in any part of the gastrointestinal tract. As used herein, the term “motility” is used to describe the contraction of the muscles that mix and propel contents in the gastrointestinal tract.

Provided herein are methods for treating or preventing constipation in a subject by administering a peptide sequence described herein to the subject. As used herein, the term “constipation” refers to a condition that is characterized by difficulty in passing stools or a low frequency in bowel movement, often accompanied by straining during defecation or a feeling of incomplete evacuation. As used herein, the term “bowel movement” refers to evacuation of feces from the gastrointestinal tract.

Bowel Function:

Constipation in a subject indicates inadequate bowel function. As used herein, the term “bowel function” refers to the ability of the intestine to absorb water and nutrients, such as fatty acid and bile acids, and evacuate wastes. Bowel function can be measured by, for example, Colonic Transit (“CT”), stool frequency and consistency, ease of passage, gastric emptying (“GE”), ascending colon empting (“AC”), fecal fat excretion, fecal bile acid excretion, or any combination thereof. Accordingly, provided herein are also methods to stimulate bowel function in a subject by administering to the subject a therapeutically effective amount of a peptide sequence disclosed herein.

As used herein, the term “colonic transit” or “CT” refers to colonic transit time, the time that it takes for a substance to move through the colon. Slow colonic transit is also known as “colonic inertia,” a condition characterized by a decreased rate or frequency of bowel activity. Colonic transit can be measured by “colonic geometric centre” or CT “GC,” which is the weighted average of counts in the colonic regions (ascending, transverse, descending, rectosigmoid) and stool, respectively, 1 to 5. At any time, the proportion of counts in each colonic region is multiplied by its weighting factor as follows: (% ascending*1+% transverse*2+% descending*3+% rectosigmoid*4+% stool*5)/100=GC. Thus, a higher CT GC reflects a faster colonic transit.

Gastric emptying or “GE” can be measured by gastric emptying half-life time (“GE t1/2”), which refers to the time require by the stomach to empty 50% of the ingested meal. As used herein, the term “gastroparesis” refers to paresis (partial paralysis) of the stomach, resulting in food remaining in the stomach for an abnormally long time. Ascending colon emptying or “AC” can be measured by ascending colon emptying half time (“AC t1/2”), which refers to the time required for emptying half of the ascending colon, calculated by linear interpolation of values on the ascending colon emptying curve.

For measuring stool frequency and consistency, and Ease of Passage, subjects record stool frequency, consistency, sense of evacuation, and ease of stool passage on daily bowel diaries during the run-in and treatment periods. Stool frequency is represented as number of Bowel Movement per week (“# BM/week”) or (“BM/week”). Ease of Passage refers to the ease of bowel movement, and can be evaluated on an adjectival scale (a 7-point adjectival scale ranging from manual disimpaction=1 to incontinence=7).

Stool consistency can be measured using the Bristol Stool Form Scale (“BSFS”). The BSFS is a standard diagnostic medical tool designed to classify the form of human feces into seven categories. The seven types of stool are:

Type 1: Separate hard lumps, like nuts (hard to pass); also known as goat faeces;
Type 2: Sausage-shaped, but lumpy;
Type 3: Like a sausage but with cracks on its surface;
Type 4: Like a sausage or snake, smooth and soft;
Type 5: Soft blobs with clear cut edges (passed easily);
Type 6: Fluffy pieces with ragged edges, a mushy stool;
Type 7: Watery, no solid pieces, entirely liquid.
BSFS Types 1 and 2 indicate constipation.

Bowel function can also be indicated by fecal fat, which refers to the amount of fact in the stool and can be measured by quantitative fecal fat test. Normally up to 7 grams of fat can be malabsorbed in people consuming 100 grams of fat per day and excreted in stool. Inadequate bowel function can result in a decrease in fat absorption and an increase in fecal fat. An increase in fecal fat can also cause changes in stool consistency.

Bowel function can also be indicated by fecal bile acids, which refers to the amount of bile acids in the stool. Two assays can be used to measure fecal bile acids. The first measures the total amount of fecal bile acids in which all of the most abundant fecal bile acids are measured separately and then added together. An elevated value of total fecal bile acids is indicative of bile acid malabsorption and inadequate bowel function. The second measures the percentage of the primary bile acids, which are the cholic acid (“CA”) and the chenodeoxycholic acid (“CDCA”), as a percent of the total fecal bile acids (“% CA+CDCA”). In subjects with inadequate bowel function, colonic transit increases, resulting in an increased rate of conversion of primary to secondary bile acids and thus a lower percentage of the primary bile acids. Accordingly, an elevated value of percentages of primary bile acids is indicative of shortened colonic transit and stimulated bowel function. Accordingly, in some embodiments, provided herein are methods for accelerating colonic transit in a subject by administering to the subject a therapeutically effective amount of a peptide sequence disclosed herein. In some embodiments, provided herein are methods for treating colonic inertia in a subject by administering to the subject a therapeutically effective amount of a peptide sequence disclosed herein. In some embodiments, provided herein are methods for increasing stool frequency in a subject by administering to the subject a therapeutically effective amount of a peptide sequence disclosed herein. In some embodiments, provided herein are methods for improving stool consistency in a subject by administering to the subject a therapeutically effective amount of a peptide sequence disclosed herein. In some embodiments, provided herein are methods for improving ease of passage in a subject by administering to the subject a therapeutically effective amount of a peptide sequence disclosed herein. In some embodiments, provided herein are methods to accelerate gastric emptying in a subject by administering to the subject a therapeutically effective amount of a peptide sequence disclosed herein. In some embodiments, provided herein are methods for treating gastroparesis in a subject by administering to the subject a therapeutically effective amount of a peptide sequence disclosed herein. In some embodiments, provided herein are methods for accelerating ascending colon empting in a subject by administering to the subject a therapeutically effective amount of a peptide sequence disclosed herein. In some embodiments, provided herein are methods for reducing fecal fat in a subject by administering to the subject a therapeutically effective amount of a peptide sequence disclosed herein.

In some embodiments, provided herein are methods for reducing fecal bile acids in a subject by administering to the subject a therapeutically effective amount of a peptide sequence disclosed herein. In some embodiments, provided herein are methods for increasing the percentage of primary bile acids in total fecal bile acids in a subject by administering to the subject a therapeutically effective amount of a peptide sequence disclosed herein.

Bowel function can also be improved in subjects in need thereof by reduction in synthesis on bile acids. Hepatic synthesis of bile acids can be reduced by reducing expression of the cytochrome P450 enzyme cholesterol 7α-hydroxylase (CYP7A1), which catalyzes the first rate-limiting step of hydroxylation of cholesterol. In some embodiments, the reduction in bile acid synthesis by the peptide is demonstrated by a reduction in levels of serum 7-alpha-hydroxy-4-cholesten-3-one (C4, a downstream product of CYP7A1 action). Luo et al., Sci Trnsl Med 6: 247ra100 (2014). Accordingly, in some embodiments, the methods described herein decreases hepatic bile acid synthesis in the subject.

In some embodiments, provided herein are methods of treating or preventing a gastrointestinal motility-related disorder caused by one or more of the factors, diseases, or medications described herein or otherwise known in the art to cause a gastrointestinal motility-related disorder. In some embodiments, provided herein are methods to treat a gastrointestinal motility-related disorder in a subject by administering a peptide sequence described herein to the subject, wherein the gastrointestinal motility-related disorder is caused by a medication. In some embodiments, provided herein are methods to treat a gastrointestinal motility-related disorder in a subject by administering a peptide sequence described herein to the subject, wherein the gastrointestinal motility-related disorder is a symptom of another disease.

Types of Constipation:

Constipation can be caused by a number of factors, such as a low-fiber diet, low liquid intake, or dieting. In some subjects, constipation can be a symptom of a systemic disease. For example, constipation is a cardinal symptom in about one third of patients with irritable bowel syndrome (“IBS”), who are known to have “constipation-predominant irritable bowel syndrome.” Metabolic and endocrine disorders are also known to cause constipation, including hypercalcemia, hypothyroidism, hyperparathyroidism, porphyria, chronic kidney disease, pan-hypopituitarism, diabetes mellitus, cystic fibrosis, and celiac disease. Also, neurological disorders can also cause constipation, including anismus, descending perineum syndrome, and Hirschsprung's disease. Constipation can also have structural (mechanical, morphological, anatomical) causes, namely through creating space-occupying lesions within the colon that stop the passage of stool, such as colorectal cancer, strictures, rectocoles, and post-surgical changes. Gastric outlet obstruction, a condition where the subject has an obstruction of the channel of the pylous and duodenum through which the stomach empties can also cause constipation. Constipation is also common in individuals with muscular and myotonic dystrophy.

In some subjects, constipation can be a side effect of a medication, such as opioid analgesics, antihypertensives, antidepressants, antipsychotics, or antiemetics. Treatments for Parkinson's disease, colonic and anorectal diseases, including carcinoma of the colon and rectum are also known to be associated with chronic constipation.

Accordingly, provided herein are methods of treating or preventing constipation caused by one or more of the factors, diseases, or medications described herein or otherwise known in the art to cause constipation. In some embodiments, provided herein are methods to treat constipation in a subject by administering a peptide sequence described herein to the subject, wherein the constipation is caused by a medication. In some embodiments, provided herein are methods to treat constipation in a subject by administering a peptide sequence described herein to the subject, wherein constipation is a symptom of another disease. In some embodiments, the constipation is a symptom of IBS. In some embodiments, the constipation is a symptom of a metabolic disorder. In some embodiments, the constipation is a symptom of endocrine disorder. In some embodiments, the constipation is a symptom of diabetes mellitus. In some embodiments, the constipation is a symptom of a neurologic disorder.

Constipation can have unknown cause. As used herein, the term “functional constipation” or “FC,” also known as “chronic idiopathic constipation” or “CIC,” refers to constipation that does not have identified physical (anatomical) or physiological (hormonal or other body chemistry) cause. According to the Rome III criteria, functional constipation is defined as the presence of two or more of the following during the previous 3 months: a) defecatory straining (≥25% bowel movements); b) hard or lumpy stools (≥25% bowel movements); c) a feeling of incomplete evacuation (≥25% bowel movements); d) defecatory obstruction (≥25% bowel movements); e) manual maneuvers to facilitate defecation (≥25% bowel movements); and f) fewer than 3 spontaneous complete bowel movements per week. Symptoms must be present for at least 6 months before the diagnosis, loose stools must not be present except after using a laxative, and IBS criteria must not be met. In some embodiments, provided herein are methods of treating functional constipation in a subject by administering to the subject a therapeutically effective amount a peptide sequence described herein.

Methods of treating a subject having a gastrointestinal motility-related disorder, with peptide sequences as described herein can ameliorate the gastrointestinal motility-related disorder by stimulating bowel function in the subject. Methods of treating a subject having constipation, or in some specific embodiments, functional constipation, with peptide sequences as described herein can ameliorate constipation by stimulating bowel function in the subject. In some embodiments, the methods described herein accelerate colonic transit (“CT”) in the subject. In some embodiments, the methods described herein increases stool frequency in the subject. In some embodiments, the methods described herein improve stool consistency in the subject. In some embodiments, the methods described herein improve ease of passage in the subject. In some embodiments, the methods described herein accelerate gastric emptying in the subject. In some embodiments, the methods described herein accelerate ascending colon empting in the subject. In some embodiments, the methods described herein reduce fecal fat in the subject. In some embodiments, the methods described herein reduce fecal bile acids in the subject. In some embodiments, the methods described herein increases the percentage of primary bile acids in total fecal bile acids in the subject. In some embodiments, the methods described herein decreases hepatic bile acid synthesis in the subject.

In some embodiments, provided herein are methods for reducing fecal bile acids in a subject by administering to the subject a therapeutically effective amount of a peptide sequence disclosed herein. In some embodiments, provided herein are methods for increasing the percentage of primary bile acids in total fecal bile acids in a subject by administering to the subject a therapeutically effective amount of a peptide sequence disclosed herein.

As described herein, the peptide sequence disclosed herein can stimulate excitatory neural control of colonic mobility by, for example, acting on KLB in neave cells. Accordingly, provided herein are methods for stimulating excitatory neural control of colonic mobility in a subject by administering to the subject a therapeutically effective amount of a peptide sequence disclosed herein.

Subjects:

As used herein, the term “subject” refers to an animal. Typically, the animal is a mammal that would benefit from treatment with a peptide sequence provided herein. Particular examples include primates (e.g., humans), dogs, cats, horses, cows, pigs, and sheep.

In some embodiments, the subject is a human.

Subjects that can be treated with methods described herein can have inadequate bowel function. Subjects that can be treated with methods described herein can have a gastrointestinal motility-related disorder. In particular embodiments, the subject has or is at risk of having a gastrointestinal motility-related disorder. In particular embodiments, the subject is a patient having a gastrointestinal motility-related disorder. Subjects at risk of developing a gastrointestinal motility-related disorder include, for example, those who may have a family history or genetic predisposition toward such disorder, as well those whose diet may contribute to development of such disorders.

Subjects that can be treated with methods described herein can have constipation. In particular embodiments, the subject has or is at risk of having constipation. In other particular embodiments, the subject has or is at risk of having gastroparesis. In other particular embodiments, the subject has or is at risk of having inadequate bowel function. Subjects at risk of developing constipation include, for example, those who may have a family history or genetic predisposition toward such disorder, as well those whose diet may contribute to development of such disorders.

In some embodiments, the subject has functional constipation, or chronic idiopathic constipation. In some embodiments, the subject has a disease associated with constipation. In some embodiments, the subject does not have constipation or a disorder known to cause constipation but is at risk of developing constipation or the disorder. In some embodiments, the subject has IBS. In some embodiments, the subject has constipation-predominant IBS. In some embodiments, the subject has a metabolic disorder. In some embodiments, the subject has an endocrine disorder. In some embodiments, the subject has gastroesophageal reflux disease. In some embodiments, the subject has intestinal dysmotility. In some embodiments, the subject has achalasia. In some embodiments, the subject has scleroderma. In some embodiments, the subject has hypercalcemia, hypothyroidism, hyperparathyroidism, porphyria, chronic kidney disease, pan-hypopituitarism, diabetes mellitus, cystic fibrosis, or celiac disease. In some embodiments, the subject has a glucose disorder. In some embodiments, the subject has a cholesterol or triglyceride metabolism disorder. In some embodiments, the subject has diabetes mellitus. In some embodiments, the subject has Type I diabetes. In some embodiments, the subject has Type II diabetes. In some embodiments, the subject has a neurological disorder. In some embodiments, the subject has anismus, descending perineum syndrome, or Hirschsprung's disease. In some embodiments, the subject has colorectal cancer, strictures, rectocoles, post-surgical changes or other reasons that create space-occupying lesions within the colon. In some embodiments, the subject has gastric outlet obstruction. In some embodiments, the subject has muscular and myotonic dystrophy.

Subjects that can be treated with methods described herein can have a bile acid-related disorder. The term “bile acid-related disorder,” or the like, when used in reference to a condition of a subject, means a disruption of bile acid homeostasis, which may manifest itself as, for example, an acute, transient or chronic abnormal level of a bile acid or one or more bile acids. The condition can be caused by inhibition, reduction or a delay in bile acid synthesis, metabolism or absorption such that the subject exhibits a bile acid level not typically found in normal subjects. Accordingly, subjects that can be treated with methods described herein can have a bile acid-related disorder, such as cholestasis, including, for example diseases of intrahepatic cholestasis (e.g., biliary cirrhosis (PBC), primary familial intrahepatic cholestasis (PFIC), primary sclerosing choangitis (PSC), pregnancy intrahepatic cholestasis (PIC), neonatal cholestasis, and drug induced cholestasis (e.g., estrogen)), and diseases of extrahepatic cholestasis (e.g., bile cut compression from tumor, bile duct blockade by gall stones); bile acid malabsorption and other disorders involving the distal small intestine, including ileal resection, inflammatory bowel diseases (e.g., Crohn's disease and ulcerative colitis), short bowel syndrome, and GI, liver, and/or biliary cancers (e.g., colon cancer and hepatocellular cancer); and/or bile acid synthesis abnormalities, such as those contributing to non-alcoholic steatohepatitis (“NASH”), nonalcoholic fatty liver disease (NAFLD), cirrhosis and portal hypertension; or subjects that do not have a disorder but may be at risk of developing the disorder. In some embodiments, the subject has cholestasis. In some embodiments, the subject has PBC. In some embodiments, the subject has PFIC. In some embodiments, the subject has PSC. In some embodiments, the subject has neonatal cholestasis. In some embodiments, the subject has PIC. In some embodiments, the subject has bile acid malabsorption. In some embodiments, the subject has NASH. In some embodiments, the subject has NAFLD. Additional bile acid-related disorders include metabolic syndrome, a lipid or glucose disorder, cholesterol or triglyceride metabolism, diabetes (e.g., type 2 diabetes), other hyperglycemic-related disorders, including kidney damage (e.g., tubule damage or nephropathy), liver degeneration, eye damage (e.g., diabetic retinopathy or cataracts), and diabetic foot disorders, and dyslipidemias and their sequelae such as, for example, atherosclerosis, coronary artery disease, cerebrovascular disorders and the like.

Other conditions associated with metabolic syndrome can also include such as obesity and elevated body mass (including the co-morbid conditions thereof such as, but not limited to, nonalcoholic fatty liver disease (NAFLD), nonalcoholic steatohepatitis (NASH), and polycystic ovarian syndrome (PCOS)), and also include thromboses, hypercoagulable and prothrombotic states (arterial and venous), hypertension (including portal hypertension (defined as a hepatic venous pressure gradient (HVPG) greater than 5 mm Hg), cardiovascular disease, stroke and heart failure; Disorders or conditions in which inflammatory reactions are involved, including atherosclerosis, chronic inflammatory bowel diseases (e.g., Crohn's disease and ulcerative colitis), asthma, lupus erythematosus, arthritis, or other inflammatory rheumatic disorders; Disorders of cell cycle or cell differentiation processes such as adipose cell tumors, lipomatous carcinomas including, for example, liposarcomas, solid tumors, and neoplasms; Neurodegenerative diseases and/or demyelinating disorders of the central and peripheral nervous systems and/or neurological diseases involving neuroinflammatory processes and/or other peripheral neuropathies, including Alzheimer's disease, multiple sclerosis, Parkinson's disease, progressive multifocal leukoencephalopathy and Guillian-Barre syndrome; Skin and dermatological disorders and/or disorders of wound healing processes, including erythemato-squamous dermatoses; and Other Disorders such as syndrome X, osteoarthritis, and acute respiratory distress syndrome.

### Prediction of Responsiveness, Patient Selection, and Companion Diagnostics

As described herein, subjects who are carriers of KLB minor allele rs17618244 have a greater response to a treatment for gastrointestinal motility-related disorder with the peptide sequence described herein. Accordingly, provided herein are also methods to predict the responsiveness of a subject having a gastrointestinal motility-related disorder to a treatment with the peptide described herein based on the presence of KLB rs17618244 minor allele, and methods to select subject having a gastrointestinal motility-related disorder for a treatment with the peptide described herein based on the presence of KLB rs17618244 minor allele.

In some embodiments, a carrier of minor allele rs17618244 has a greater acceleration of colonic transit than a non-carrier when treated with a peptide sequence described herein. In some embodiments, a carrier of minor allele rsl 7618244 has a greater improvement of colonic inertia than a non-carrier when treated with a peptide sequence described herein. In some embodiments, a carrier of minor allele rs17618244 has a greater increase in stool frequency than a non-carrier when treated with a peptide sequence described herein. In some embodiments, a carrier of minor allele rs17618244 has a greater improvement in stool consistency than a non-carrier when treated with a peptide sequence described herein. In some embodiments, a carrier of minor allele rs17618244 has a greater acceleration of colonic transit than a non-carrier when treated with a peptide sequence described herein. In some embodiments, a carrier of minor allele rs17618244 has a greater improvement in ease of passage than a non-carrier when treated with a peptide sequence described herein. In some embodiments, a carrier of minor allele rs17618244 has a greater acceleration of gastric emptying than a non-carrier when treated with a peptide sequence described herein. In some embodiments, a carrier of minor allele rs17618244 has a greater improvement in gastroparesis than a non-carrier when treated with a peptide sequence described herein. In some embodiments, a carrier of minor allele rs17618244 has a greater acceleration of ascending colon empting than a non-carrier when treated with a peptide sequence described herein. In some embodiments, a carrier of minor allele rs17618244 has a greater reduction in fecal fat than a non-carrier when treated with a peptide sequence described herein.

Accordingly, provided herein are also methods to predict the responsiveness of a subject having a gastrointestinal motility-related disorder to the treatment with a peptide sequence described herein, comprising genotyping the subject, wherein the subject who is a carrier of KLB minor allele rs17618244 is predicted to have a greater response to the treatment than a non-carrier. In some embodiments, provided herein are also methods to select a subject having a gastrointestinal motility-related disorder for the treatment with a peptide sequence described herein, comprising genotyping the subject, wherein the subject who is a carrier of KLB minor allele rs17618244 is selected for the treatment.

In some embodiments, provided herein are methods to of treating a subject having a gastrointestinal motility-related disorder comprising first genotyping the subject to determine the presence of the KLB minor allele rs17618244, and administering a therapeutically effective amount of a peptide sequence described herein to the subject who is a carrier of the KLB minor allele rs17618244.

In some embodiments, the subject is heterozygous for KLB minor allele rs17618244. In some embodiments, the subject is homozygous for KLB minor allele rs17618244.

The peptide sequence can be any peptide sequence exemplified in Table 1, Sequence Listing, or otherwise described in this application. In some embodiments, the peptide sequence is M69. In some embodiments the peptide sequence is M70. In some embodiments, the peptide sequence is fused with a Fc region. In some embodiments, the peptide sequence is M69 fused a human antibody Fc fragment. In some embodiments, the peptide sequence is M70 fused a human antibody Fc fragment.

In some embodiments, genotyping a subject can be performed using Single Nucleotide Polymorphism (SNP) assay, restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, DNA microarrays, Mass Spectrometry (MS), or denaturing high-performance liquid chromatography (DHPLC). Methods of genotyping also include whole genome sequencing. Other methods of genotyping are also known in the art and can be used in the current invention. A person of ordinary skill in the art would understand that any genotyping technologies described herein or otherwise known in the art can be used.

Provided herein are also methods of of treating a subject having a gastrointestinal motility-related disorder comprising (A) providing a sample from the subject; (B) selecting the patient for treatment based on the presence of the KLB minor allele rs17618244 in the sample; and (C) administering a therapeutically effective amount of a peptide sequence described herein to the subject.

Provided herein are also methods of treating a subject having a gastrointestinal motility-related disorder comprising (A) obtaining a sample from the subject; (B) detecting the presence of the KLB minor allele rs17618244 in the sample; and (C) diagnosing the subject as highly sensitive to a peptide sequence if the KLB minor allele rs17618244 is present in the sample; and (D) administering a therapeutically effective amount of the peptide sequence described herein to the subject.

Provided herein are also methods of treating a subject having a gastrointestinal motility-related disorder comprising (A) obtaining a sample from the subject; (B) detecting the presence of the KLB minor allele rs17618244 in the sample; (C) selecting the subject as highly sensitive to a peptide sequence if the KLB minor allele rs17618244 is present in the sample; and (D) administering a therapeutically effective amount of the peptide sequence described herein to the subject.

Provided herein are also methods determining the responsiveness of a subject having a gastrointestinal motility-related disorder to a treatment, comprising (A) obtaining a sample from the subject; (B) determining the presence of the KLB minor allele rs17618244 in the sample; and (C) diagnosing the subject as highly sensitive to the treatment if the KLB minor allele rs17618244 is present in the sample; wherein the treatment comprises administering a therapeutically effective amount of a peptide sequence described herein to the subject.

In some embodiments, determining the presence of the KLB minor allele rs17618244 includes detecting the presence of a complex or the formation of a reaction product in a sample from the subject having a gastrointestinal motility-related disorder, wherein the complex or reaction product indicates the presence of the KLB minor allele rs17618244. In some embodiments, the complex is detectably labeled. In some embodiments, the reaction product is detectably labeled. In some embodiments, the complex is a hybridization complex. In some embodiments, the hybridization complex is attached to a solid support.

Provided herein are also methods of identifying a subject having a gastrointestinal motility-related disorder as highly sensitive to a treatment, comprising (A) detecting the presence of a complex or the formation of a reaction product in a sample from the subject, wherein the complex or reaction product indicates the presence of the KLB minor allele rs17618244 in the sample; and (B) diagnosing the subject as highly sensitive to the based on the presence of the complex or reaction product in the sample; wherein the treatment comprises administering a therapeutically effective amount of a peptide sequence described herein to the subject.

Provided herein are also methods of predicting the responsiveness of a subject having a gastrointestinal motility-related disorder to a treatment, comprising detecting a complex or the formation of a reaction product in a sample from the subject, wherein the presence of the complex or reaction product indicates the presence of KLB minor allele rs17618244, and wherein the subject is predicted to have a greater response to the treatment than a non-carrier of KLB minor allele rs17618244; wherein the treatment comprises administering a therapeutically effective amount of a peptide sequence described herein to the subject.

Provided herein are also methods of determining the responsiveness of a subject having a gastrointestinal motility-related disorder to a treatment, comprising (A) obtaining a sample from the subject; (B) determining the presence of the KLB minor allele rs17618244 in the sample by detecting the presence of a complex or the formation of a reaction product, wherein the complex or reaction product indicates the presence of the KLB minor allele rs17618244 in the sample; and (C) diagnosing the subject as highly sensitive to the treatment if the KLB minor allele rs17618244 is present in the sample; wherein the treatment comprises administering a therapeutically effective amount of a peptide sequence to the subject; wherein the treatment comprises administering a therapeutically effective amount of a peptide sequence described herein to the subject.

In some embodiments, the complex is detectably labeled. In some embodiments, the reaction product is detectably labeled. Exemplary labels include, but are not limited to, radioactive isotopes, magnetic beads, metallic beads, colloidal particles, fluorescent dyes, enzymes, biotin, digoxigenin, haptens, and the like. In some embodiments, the complex is a hybridization complex. In some embodiments, the hybridization complex is attached to a solid support.

A solid support can be flat (planar), although as will be appreciated by those in the art, other configurations of substrates may be used as well; for example, three dimensional configurations can be used, for example by embedding beads in a porous block of plastic that allows sample access to the beads and using a confocal microscope for detection. Similarly, the beads may be placed on the inside surface of a tube, for flow-through sample analysis to minimize sample volume. In some aspects substrates include optical fiber bundles and flat planar substrates such as glass, polystyrene and other plastics and acrylics. A bead includes a small discrete particle, the composition of which will depend on the class of probe used and the method of synthesis. Suitable bead compositions include those used in peptide, nucleic acid and organic moiety synthesis, including, but not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and Teflon® may all be used. “Microsphere Detection Guide” from Bangs Laboratories, Fishers Ind. is a helpful guide.

In some embodiments, the subject who is a carrier of the KLB minor allele rs17618244 is heterozygous for KLB minor allele rs17618244. In some embodiments, the subject who is a carrier of the KLB minor allele rs17618244 is homozygous for KLB minor allele rs17618244.

The peptide sequence can be any peptide sequence exemplified in Table 1, Sequence Listing, or otherwise described in this application. In some embodiments, the peptide sequence is M69. In some embodiments the peptide sequence is M70. In some embodiments, the peptide sequence is fused with a Fc region. In some embodiments, the peptide sequence is M69 fused a human antibody Fc fragment. In some embodiments, the peptide sequence is M70 fused a human antibody Fc fragment.

In certain embodiments, detection of the KLB minor allele rs17618244 employs methods and/or techniques that are well known to those of ordinary skill in the art. Non-limiting examples of suitable detection methods and/or techniques include, but are not limited to, hybrization based methods (e.g., dynamic allele-specific hybrization, hybrization with a molecular beacon, or SNP microarrays), enzyme-based methods (e.g., restriction fragment length polymorphism (RFLP), polymerase chain reaction (PCR) based methods, flap endonuclease (FEN), primer extension methods, DNA polymerase with 5′-nuclease activity methods, or oligonucleotide ligation assays), physical property assays (e.g., single strand conformational polymorphism gel electrophoresis, temperature gradient gel electrophoresis, denaturing high performance liquid chromatography (DHPLC), high resolution melting analysis, or DNA mismatch-binding protein electrophoresis) and DNA sequencing (e.g., pyrosequencing, single-molecule real-time sequencing, ion torrent sequencing, sequencing by synthesis, sequencing by ligation, chemical degradation sequencing (Maxam-Gilbert sequencing), or chain termination sequencing (Sanger sequencing)). One of ordinary skill in the art would be able to readily ascertain the appropriate method based on the patient sample and perform such methods.

One suitable method for identifying or detecting KLB minor allele rs17618244 is by sequencing a part of or all of a gene (full-length sequencing).

Sequencing can be performed using any art recognized method. An exemplary sequencing method is the Sanger method using chain-termination chemistry. However, next generation sequencing methods are evolving quickly and are capable of sequencing very large areas of the genome rapidly. For deletions, insertions, and SNPs, both Sanger sequencing and next-generation sequencing methods can be applied. Next-generation sequencing techniques offer the ability to sequence entire genes in one run making these technique preferable when trying to discover new polymorphisms. For already known SNPs, such as those described herein, TaqMane, molecular beacons, RFLP, qPCR, and various bead-based techniques are well-suited.

Genotyping of SNPs can be performed by any art recognized method. For example, genomic DNA can be purified from a sample from a patient, and then the genomic DNA is analyzed to genotype one or more SNPs. With multiplex assays, for example, it is possible to analyze a number of SNPs and patients simultaneously. A variety of technologies have been developed for SNP analysis. Most methods are amplification based and the SNP is subsequently detected by primer extension, oligonucleotide ligation, or hybridization of a probe to the amplified product.

In certain embodiments of the various methods provided herein, the method further comprises amplification of the DNA comprising the KLB rs17618244. In some embodiments, the amplification comprises PCR. In certain embodiments, the primer for use in the amplification includes an allele specific primer. In some embodiments, the primers are detectably labeled. In one embodiment, the methods provided herein further comprise: i) a PCR reaction in which the KLB rs17618244 SNP is amplified, ii) an allele-specific primer extension reaction (ASPE) in which detectable labels are incorporated into the ASPE-primers which match the genotype of the sample, iii) isolating the extension reaction products into separate populations of individual SNP amplification products.

In one embodiment, the detectable label is a biotin label, such as a biotinylated nucleotide. Further alternative detectable labels include phycoerythrin (PE)-labeled moieties (such as nucleotides). Alternatively, one could use a radio-labeled moiety.

In some embodiments, the methods comprise a hybridization based isolation of individual populations of SNP amplification products, such as bead-array hybridization.

It will be recognized that alternative methods of labeling the multiplex products can be used in the methods provided herein other than ASPE. Non-limiting examples include single base chain extension (SBCE), Oligonucleotide ligation assay (OLA), or alternatively the PCR products may be directly hybridized to (SNP specific) probe-coupled beads based on the presence or absence of the SNP.

Single base chain extension differs from ASPE in several ways. The allele-specific primers 3′-ends overlap one of the nucleotides located right next to the SNP-loci on either the 3′- or the 5′-side of the SNP. When an allele-specific primer hybridizes to a SNP-locus the polymerase elongates it incorporating a biotinylated dideoxy-dNTP (ddNTP). This method has the advantage that a single allele-specific primer can be used to detect up to four different alleles at a given locus; however the reaction has to be performed in four different tubes corresponding to the four possible nucleotides ddATP, ddCTP, ddGTP and ddTTP.

Oligonucleotide Ligation Assay (OLA):

The OLA-assay is based on the ability of two oligonucleotides, one labeled the other allele-specific, to anneal immediately adjacent to each other on a complementary target DNA molecule. The two oligonucleotides are then joined covalently by the action of a DNA ligase, provided that the nucleotides at the junction are correctly base-paired. In this way only a primer matching the present allele at a polymorphic locus will be joined to the labeled oligonucleotide and hence emit detectable fluorescence.

Probe-Bead Based Assay:

In the probe-bead based assay a multiplex PCR is performed on the SNP-sites of interest with at least one of the primers in each primer-pair being labeled. An allele-specific probe overlapping a suitable area of the polymorphic locus is then prepared and coupled covalently to suitable microspheres. With all other than the perfectly matching PCR-product, the probe will form a loop because of the mismatching base-pair in the middle of the probe-PCR product hybridization complex and this significantly decreases the melting temperature of the complex ensuring that only perfectly hybridized oligonucleotides will remain attached to the probe and hence emit detectable fluorescence.

ASPE, SBCE, OLA and the probe-bead based assays are all suited for an Illumina platform as exemplified herein, but different solid base supports such as microarray chips or other beads available for FACS-cytometers, etc. could be substituted for the Illumina platform.

Other exemplary SNP detection methods can be used in the the methods provided herein. Exemplary methods are described in Chen et al., Pharmacogenomics J. 3(2):77-96 (2003); Kwok et al., Curr. Issues Mol. Biol. 5(2):43-60 (2003); Shi, Am. J. Phannacogenomics 2(3):197-205 (2002); and Kwok, Annu. Rev. Genomics Hum. Genet. 2:235-58 (2001). Exemplary techniques for high-throughput SNP detection are described in Marnellos, Curr. Opin. Drug Discov. Devel. 6(3):317-21 (2003). Other SNP detection methods useful in the methods provided herein include, but are not limited to, TaqMan® assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Provided herein are also kits for predicting the responsiveness of a subject to a treatment for a gastrointestinal motility-related disorder using a peptide sequence disclosed herein, comprising at least one agent for determining the presence or absence of KLB minor allele rs17618244. The kits provided herein can include an ancillary agent.

In some embodiments, the kits further include reagents for genomic DNA isolation or purification means, detection means, as well as positive and negative controls. In certain embodiments, the kits further include instructions for users. In some embodiments, the kits further include a peptide sequence described herein or a pharmaceutical composition having a peptide sequence described herein. The kits can be tailored for in-home use, clinical use, or research use. In some embodiments, the kits further include a label describing its use as a companion diagnostic to predict the responsiveness of a subject to the treatment of a gastrointestinal motility-related disorder using a peptide sequence disclosed herein.

The peptide sequence can be any peptide sequence exemplified in Table 1, Sequence Listing, or otherwise described in this application. In some embodiments, the peptide sequence is M69. In some embodiments the peptide sequence is M70. In some embodiments, the peptide sequence is fused with a Fc region. In some embodiments, the peptide sequence is M69 fused a human antibody Fc fragment. In some embodiments, the peptide sequence is M70 fused a human antibody Fc fragment.

The kits provided herein can employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide.

In some embodiments, such kits can include, a computer program product embedded on computer readable media for predicting the responsiveness of a subject to a treatment for a gastrointestinal motility-related disorder using a peptide sequence disclosed herein. In some embodiments, the kits can include a computer program product embedded on a computer readable media along with instructions.

In some embodiments, the kits provided herein include agent for genotyping a subject using Single Nucleotide Polymorphism (SNP) assay, restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, DNA microarrays, Mass Spectrometry (MS), or denaturing high-performance liquid chromatography (DHPLC). For nucleic acid microarray kits, the kits generally include probes attached to a solid support surface. In one such embodiment, probes can be either be oligonucleotides or longer length probes including probes ranging from 150 nucleotides in length to 800 nucleotides in length. The probes can be attached to a detectable label. In a specific embodiment, the probes are specific for the KLB minor allele rs17618244. The microarray kits can include instructions for performing the assay and methods for interpreting and analyzing the data resulting from the performance of the assay. Generally, the materials and reagents for the microarray kits are in one or more containers. Each component of the kit is generally in its own a suitable container.

In certain embodiments of the methods and kits provided herein, solid phase supports are used for purifying gDNA, labeling samples or carrying out the solid phase assays. Examples of solid phases suitable for carrying out the methods disclosed herein include beads, particles, colloids, single surfaces, tubes, multiwell plates, microtiter plates, slides, membranes, gels and electrodes. When the solid phase is a particulate material (e.g., beads), it is, in one embodiment, distributed in the wells of multi-well plates to allow for parallel processing of the solid phase supports.

Kits provided herein can further include devices that are used to administer the peptide sequence described herein or other active ingredients. Examples of such devices include, but are not limited to, syringes, drip bags, patches, and inhalers. Kits can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The kit of this disclosure can include an ancillary reagent. In some embodiments, the ancillary reagent can be a detection reagent, a detection buffer, an immobilization buffer, a dilution buffer, a washing buffer, or any combination thereof.

Any detection reagent known in the art can be included in a kit of this disclosure. In some embodiments, the detection reagent is a colorimetric detection reagent, a fluorescent detection reagent, or a chemiluminescent detection reagent. In some embodiments, the colorimetric detection reagent includes PNPP (p-nitrophenyl phosphate), ABTS (2,2′-azino-bis(3-ethylbenzothiazoline-6-sulphonic acid)) or OPD (o-phenylenediamine). In some embodiments, the fluorescent detection reagent includes QuantaBlu™ or QuantaRed™ (Thermo Scientific, Waltham, Mass.). In some embodiments, the luminescent detection reagent includes luminol or luciferin. In some embodiments, the detection reagent includes a trigger (e.g., H2O2) and a tracer (e.g., isoluminol-conjugate).

Any detection buffer known in the art can be included in a kit of this disclosure. In some embodiments the detection buffer is a citrate-phosphate buffer (e.g., about pH 4.2).

Any stop solution known in the art can be included in a kit of this disclosure. The stop solutions of this disclosure terminate or delay the further development of the detection reagent and corresponding assay signals. Stop solutions can include, for example, low-pH buffers (e.g., glycine-buffer, pH 2.0), chaotrophic agents (e.g., guanidinium chloride, sodium-dodecylsulfate (SDS)) or reducing agents (e.g., dithiothreitol, mecaptoethanol), or the like.

In some embodiments, the ancillary reagent is an immobilization reagent, which can be any immobilization reagent known in the art, including covalent and non-covalent immobilization reagents. Covalent immobilization reagents can include any chemical or biological reagent that can be used to covalently immobilize a peptide or a nucleic acid on a surface. Covalent immobilization reagents can include, for example, a carboxyl-to-amine reactive group (e.g., carbodiimides such as EDC or DCC), an amine reactive group (e.g., N-hydroxysuccinimide (NHS) esters, imidoesters), a sulfhydryl-reactive crosslinker (e.g., maleimides, haloacetyls, pyridyl disulfides), a carbonyl-reactive crosslinker groups (e.g., hydrazides, alkoxyamines), a photoreactive crosslinker (e.g., aryl azides, dizirines), or a chemoselective ligation group (e.g., a Staudinger reaction pair). Non-covalent immobiliazation reagents include any chemical or biological reagent that can be used to immobilize a peptide or a nucleic acid non-covalently on a surface, such as affinity tags (e.g., biotin) or capture ragents (e.g., streptavidin or anti-tag antibodies, such as anti-His6 or anti-Myc antibodies).

The kits of this disclosure can include combinations of immobilization reagents. Such combinations include, for example, EDC and NHS, which can be used, for example, to immobilize a protein of this disclosure on a surface, such as a carboxylated dextrane matrix (e.g., on a BIAcore™ CM5 chip or a dextrane-based bead). Combinations of immobilization reagents can be stored as premixed reagent combinations or with one or more immobilization reagents of the combination being stored separately from other immobilization reagents.

A large selection of washing buffers are known in the art, such as tris(hydroxymethyl)aminomethane (Tris)-based buffers (e.g., Tris-buffered saline, TBS) or phosphate buffers (e.g., phosphate-buffered saline, PBS). Washing buffers can include detergents, such as ionic or non-ionic detergents. In some embodiments, the washing buffer is a PBS buffer (e.g., about pH 7.4) including Tween®20 (e.g., about 0.05% Tween®20).

Any dilution buffer known in the art can be included in a kit of this disclosure. Dilution buffers can include a carrier protein (e.g., bovine serum albumin, BSA) and a detergent (e.g., Tween®20). In some embodiments, the dilution buffer is PBS (e.g., about pH 7.4) including BSA (e.g., about 1% BSA) and Tween®20 (e.g., about 0.05% Tween®20).

In some embodiments, the kit of this disclosure includes a cleaning reagent for an automated assay system. An automated assay system can include systems by any manufacturer. In some embodiments, the automated assay systems include, for example, the BIO-FLASH™, the BEST 2000™, the DS2™, the ELx50 WASHER, the ELx800 WASHER, and the ELx800 READER. A cleaning reagent can include any cleaning reagent known in the art.

In a particular embodiment, provided herein is a kit that includes an agen for predicting the responsiveness of a subject having constipation to the treatment by M69 or M70, comprising an agent to determine KLB SNP rs17618244, and an ancillary agent.

It is noted that any combination of the above-listed embodiments, for example, with respect to one or more reagents, such as, without limitation, nucleic acid primers, solid support and the like, are also contemplated in relation to any of the various methods and/or kits provided herein.

### Dosing and Administration

As disclosed herein, treatment methods include administering a peptide as set forth herein (e.g., a variant or fusion of FGF19 and/or FGF21 as set forth in the Sequence Listing or Table 1) in an amount effective to achieve a desired outcome or result in a subject. A treatment that results in a desired outcome or result includes decreasing, reducing or preventing the severity or frequency of one or more symptoms of the condition in the subject, e.g., an improvement in the subject's condition or a “beneficial effect” or “therapeutic effect.” Therefore, treatment can decrease or reduce or prevent the severity or frequency of one or more symptoms of the disorder, stabilize or inhibit progression or worsening of the disorder, and in some instances, reverse the disorder, transiently (e.g., for 1-6, 6-12, or 12-24 hours), for medium term (e.g., 1-6, 6-12, 12-24 or 24-48 days) or long term (e.g., for 1-6, 6-12, 12-24, 24-48 weeks, or greater than 24-48 weeks). Thus, in the case of a gastrointestinal motility-related disorder, treatment can lower or reduce one or more symptoms or effects of the gastrointestinal motility-related disorder.

An “effective amount” or a “sufficient amount” for use and/or for treating a subject refers to an amount that provides, in single or multiple doses, alone, or in combination with one or more other agents, treatments, protocols, or therapeutic regimens, a detectable response of any duration of time (transient, medium or long term), a desired outcome in or an objective or subjective benefit to a subject of any measurable or detectable degree or for any duration of time (e.g., for hours, days, months, years, in remission or cured). Such amounts typically are effective to ameliorate a disorder, or one, multiple or all adverse symptoms, consequences or complications of the disorder, to a measurable extent, although reducing or inhibiting a progression or worsening of the disorder, is considered a satisfactory outcome.

As used herein, the term “ameliorate” means an improvement in the subject's disorder, a reduction in the severity of the disorder, or an inhibition of progression or worsening of the disorder (e.g., stabilizing the disorder). In the case of a gastrointestinal motility-related disorder, an improvement can be a lowering or a reduction in one or more symptoms or effects of the disorder.

A therapeutic benefit or improvement therefore need not be complete ablation of any one, most or all symptoms, complications, consequences or underlying causes associated with the disorder or disease. Thus, a satisfactory endpoint is achieved when there is a transient, medium or long term, incremental improvement in a subject's condition, or a partial reduction in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of the disorder or disease, over a duration of time (hours, days, weeks, months, etc.).

Thus, in the case of a disorder treatable by a peptide sequence provided herein, the amount of the peptide and the additional agent sufficient to ameliorate a disorder will depend on the type, severity and extent, or duration of the disorder, the therapeutic effect or outcome desired, and can be readily ascertained by the skilled artisan. Appropriate amounts will also depend upon the individual subject (e.g., the bioavailability within the subject, gender, age, etc.). For example, a transient, or partial, restoration of normal bowel function in a subject can reduce the dosage amount or frequency of the peptides described herein in order to treat the gastrointestinal motility-related disordereven though complete freedom from treatment has not resulted. A therapeutically effective amount can be ascertained, for example, by measuring one or more relevant physiological effects.

Peptide sequences provided herein including subsequences, sequence variants and modified forms of the exemplified peptide sequences (e.g., sequences listed in the Sequence Listing or Table 1), may be formulated in a unit dose or unit dosage form. In a particular embodiment, a peptide sequence is in an amount effective to treat a subject in need of treatment. Exemplary unit doses range from about 25-250, 250-500, 500-1000, 1000-2500 or 2500-5000, 5000-25,000, 25,000-50,000 ng; from about 25-250, 250-500, 500-1000, 1000-2500 or 2500-5000, 5000-25,000, 25,000-50,000 μg; and from about 25-250, 250-500, 500-1000, 1000-2500 or 2500-5000, 5000-25,000, 25,000-50,000 mg.

Peptide sequences provided herein including subsequences, sequence variants and modified forms of the exemplified peptide sequences (e.g., sequences listed in the Sequence Listing or Table 1) can be administered to provide the intended effect as a single dose or multiple dosages, for example, in an effective or sufficient amount. Exemplary doses range from about 25-250, 250-500, 500-1000, 1000-2500 or 2500-5000, 5000-25,000, 25,000-50,000 pg/kg; from about 50-500, 500-5000, 5000-25,000 or 25,000-50,000 ng/kg; and from about 25-250, 250-500, 500-1000, 1000-2500 or 2500-5000, 5000-25,000, 25,000-50,000 μg/kg. Single or multiple doses can be administered, for example, multiple times per day, on consecutive days, alternating days, weekly or intermittently (e.g., twice per week, once every 1, 2, 3, 4, 5, 6, 7 or 8 weeks, or once every 2, 3, 4, 5 or 6 months).

Peptide sequences provided herein including subsequences, variants and modified forms of the exemplified peptide sequences (e.g., sequences listed in the Sequence Listing or Table 1) can be administered and methods may be practiced via systemic, regional or local administration, by any route. For example, a peptide sequence can be administered parenterally (e.g., subcutaneously, intravenously, intramuscularly, or intraperitoneally), orally (e.g., ingestion, buccal, or sublingual), inhalation, intradermally, intracavity, intracranially, transdermally (topical), transmucosally or rectally. Peptide sequences provided herein including subsequences, variants and modified forms of the exemplified peptide sequences (e.g., sequences listed in the Sequence Listing or Table 1) and methods provided herein including pharmaceutical compositions can be administered via a (micro)encapsulated delivery system or packaged into an implant for administration.

A particular non-limiting example of parenteral (e.g., subcutaneous) administration entails the use of Intarcia's subcutaneous delivery system (Intarcia Therapeutics, Inc.; Hayward, Calif.). The system comprises a miniature osmotic pump that delivers a consistent amount of a therapeutic agent over a desired period of time. In addition to maintaining drug levels within an appropriate therapeutic range, the system can be used with formulations that maintain the stability of proteinaceous therapeutic agents at human body temperature for extended periods of time.

Another non-limiting example of parenteral administration entails the use of DUROS®-type implantable osmotic pumps (from, e.g., DURECT Corp.). The DUROS® system can be used for therapies requiring systemic or site-specific administration of a drug. To deliver drugs systemically, the DUROS® system is placed just under the skin, for example in the upper arm, in an outpatient procedure that is completed in just a few minutes using local anesthetic. To deliver a drug to a specific site, miniaturized catheter technology can be used. The catheter can be attached to the DUROS® system to direct the flow of a drug to the target organ, tissue or synthetic medical structure, such as a graft. Site-specific delivery enables a therapeutic concentration of a drug to be administered to the desired target without exposing the entire body to a similar concentration. The precision, size and performance of the DUROS® system will allow for continuous site-specific delivery to a variety of precise locations within the body.

Yet another non-limiting example of parenteral administration entails the use of an on-body delivery system (e.g., the NEULASTA® Delivery Kit by Amgen). This on-body delivery system includes an on-body injector, which is a small, lightweight, injection system applied on the same day as a doctor visit (such as the day of chemotherapy). It is designed to deliver a dose of the therapeutic agent the next day, or in the near future of the doctor visit, so that the patient does not need to return to the doctor's office to receive the injection.

Various methods of controlled release is also contemplated herein. Encapsulation of therapeutic molecules within polymer particles is a well-established method for achieving controlled release and can be used in methods provided herein. Also, by taking advantage of the adsorption of protein therapeutics to poly(lactic-co-glycolic acid) (PLGA) nanoparticles, controlled release can also be achieved without encapsulation. In particular, extended-release for protein therapeutics cam be applied with and without encapsulation in PLGA nanoparticles embedded within a hydrogel. The release profile tunable by modifying nanoparticle concentration, nanoparticle size, or environmental pH. Pakulska et at, Science Advances 2(5): e1600519 (2016).

### Combination Therapy

Also provided herein is the use of variants of FGF19 peptide sequences, fusions of FGF19 and/or FGF21 peptide sequences and variants of fusions (chimeras) of FGF19 and/or FGF21 peptide sequences having one or more activities associated with the treatment and/or prevention of a gastrointestinal motility-related disorder, in combination with other therapeutic agents and/or treatment modalities. Provided herein is the use of variants of FGF19 peptide sequences, fusions of FGF19 and/or FGF21 peptide sequences and variants of fusions (chimeras) of FGF19 and/or FGF21 peptide sequences having one or more activities associated with the treatment and/or prevention of constipation, in combination with other therapeutic agents and/or treatment modalities.

By way of example, additional agents (and classes thereof) that can be used in combination with peptide sequences described herein in the methods of treating a gastrointestinal motility-related disorder, such as constipation, or in the methods of stimulating bowel function include, but are not limited to bulk-forming agents (fibers), emollient stool softeners, rapidly acting lubricants, prokinetics, laxatives, osmotic agents, and prosecretory drugs.

Bulk-forming agents can be used for long-term prophylaxis and/or treatment of constipation in patients without anatomic outlet obstruction. Bulk-forming agents include for example syllium (METAMUCIL, FIBERALL, BULK-K, FIBRO-XL) and Methylcellulose (CITRUCEL).

Emollient stool softeners cause stool to soften, which are used for prophylaxis against constipation in acute and subacute settings. Emollient stool softeners include, for example, Docusate (COLACE, CORRECTOL, DOCU-SOFT, DOK).

Stimulants can increase the peristaltic activity in the gastrointestinal (GI) system. Stimulants include, for example, Senna concentrate/docusate (PERI-COLACE, DOK PLUS, SENOKOT-S).

Saline laxatives are used for acute treatment of constipation in the absence of bowel obstruction. Saline laxatives include, for example, Magnesium hydroxide (PHILLIPS MILK OF MAGNESIA, FLEET PEDIA-LAX chewable), Magnesium citrate (CITROMA), and Magnesium sulfate.

Stimulant laxatives are commonly employed to treat acute constipation and are the most common class of laxatives used over the long term by individuals taking over-the-counter products. Stimulant laxatives include, for example, Senna (SENOKOT, EX-LAX, SENEXON, SENNA-GEN), BISACODYL (BISAC-EVAC, BISCOLAX, DULCOLAX, DACODYL), Cascara sagrada, Castor oil.

Lubricant laxatives lubricate the intestine and facilitate passage of stool by decreasing water absorption from the intestine, which are used for acute or subacute management of constipation. Lubricant laxatives include, for example, Mineral oil (FLEET, KONDREMUL)

Other laxatives can elicit various pharmacologic effects resulting in increased intestinal fluid and thereby decrease constipation symptoms, including for example, Lubiprostone (AMITIZA), Linaclotide (LINZESS), and Plecanatide (TRULANCE).

Osmotic agents are useful for long-term treatment of constipated patients with slow colonic transit who are refractory to dietary fiber supplementation. Osmotic agents include, for example, Lactulose (CONSTULOSE, ENULOSE, GENERLAC, KRISTALOSE), and Polyethylene glycol solution (MIRALAX).

Prokinetics are promotility agents proposed for use in patients with severe constipation-predominant symptoms. Prokinetics include, for example, Tegaserod (ZELNORM).

Peripherally acting mu-opioid receptor antagonists (“PAMORAs”) can provide relief from GI adverse effects such as constipation associated with chronic opioid use. PAMORAs include, for example, Methylnaltrexone (RELISTOR), Naloxegol (MOVANTIK), Alvimopan (ENTEREG), and Naldemedine (SYMPROIC).

Accordingly, treatment methods can include administering one or more additional agents or therapeutic modalities useful in the treatment or prevention of a gastrointestinal motility-related disorder, such as constipation, or in stimulating bowel function, such as those agents or therapeutic modalities described herein, in an amount effective to achieve a desired outcome or result in a subject. A treatment that results in a desired outcome or result includes decreasing, reducing or preventing the severity or frequency of one or more symptoms of the condition in the subject, e.g., an improvement in the subject's condition or a “beneficial effect” or “therapeutic effect.” Therefore, treatment can decrease or reduce or prevent the severity or frequency of one or more symptoms of the disorder, stabilize or inhibit progression or worsening of the disorder, and in some instances, reverse the disorder, transiently (e.g., for 1-6, 6-12, or 12-24 hours), for medium term (e.g., 1-6, 6-12, 12-24 or 24-48 days) or long term (e.g., for 1-6, 6-12, 12-24, 24-48 weeks, or greater than 24-48 weeks). Thus, in the case of a gastrointestinal motility-related disorder, treatment with a peptide provided herein in combination with another therapeutic agent can lower or reduce one or more symptoms or effects of the gastrointestinal motility-related disorder In the case of constipation, treatment with a peptide provided herein in combination with another therapeutic agent can lower or reduce one or more symptoms or effects of constipation.

Accordingly, methods and uses provided herein for treating a subject having, or at risk of developing, a gastrointestinal motility-related disorder can be practiced prior to, substantially contemporaneously with, or following administration or application of another agent useful for the treatment or prevention of the gastrointestinal motility-related disorder, and/or can be supplemented with other forms of therapy. Methods and uses provided herein for treating a subject having, or at risk of developing, constipation or gastroparesis can be practiced prior to, substantially contemporaneously with, or following administration or application of another agent useful for the treatment or prevention of constipation, and/or can be supplemented with other forms of therapy.

Supplementary therapies can be administered prior to, contemporaneously with or following methods and uses provided herein.

### Compositions

Also provided herein are “pharmaceutical compositions,” which include a peptide sequence (or sequences) provided herein, including subsequences, variants and modified forms of the exemplified peptide sequences (e.g., sequences listed in the Sequence Listing or Table 1), and one or more pharmaceutically acceptable or physiologically acceptable diluents, carriers or excipients; in combination with, or separate from, one or more additional agents for the treatment or prevention of a gastrointestinal motility-related disorder, for the treatment or prevention of constipation or for the stimulation of bowel function, or a composition comprising such one or more additional agents and one or more pharmaceutically acceptable or physiologically acceptable diluents, carriers or excipients. In particular embodiments, a peptide sequence or sequences and an additional agent(s) are present in a therapeutically acceptable amount. The pharmaceutical compositions can be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some aspects, the pharmaceutical compositions may further comprise an additional therapeutically active agents or compounds disclosed or known to the skilled artisan which can be used in the treatment or prevention of a gastrointestinal motility-related disorder as set forth herein. In some aspects, the pharmaceutical compositions can further comprise an additional therapeutically active agents or compounds disclosed or known to the skilled artisan which can be used in the treatment or prevention of constipation as set forth herein. For example, the addition agent can be a bulk-forming agent (fiber), an emollient stool softener, a lubricant, a prokinetic, a laxative, an osmotic agents, or a prosecretory drug. As set forth above, the additional therapeutically active agents or compounds can be present in a separate pharmaceutical composition(s). Exemplary dosing parameters and regimens are described herein.

Pharmaceutical compositions typically comprise a therapeutically effective amount of at least one of the peptide sequences provided herein, including subsequences, variants and modified forms of the exemplified peptide sequences (e.g., sequences listed in the Sequence Listing or Table 1) and/or one or more additional agents described herein, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that could be used in the pharmaceutical compositions and dosage forms used herein. Typical buffers include, but are not limited to pharmaceutically acceptable weak acids, weak bases, or mixtures thereof. Buffer components also include water soluble materials such as phosphoric acid, tartaric acids, lactic acid, succinic acid, citric acid, acetic acid, ascorbic acid, aspartic acid, glutamic acid, and salts thereof.

A primary solvent in a vehicle may be either aqueous or non-aqueous in nature. In addition, the vehicle may contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein may contain still other pharmaceutically-acceptable formulation agents for modifying or maintaining the rate of release of a peptide and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, N.J.); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

A pharmaceutical composition may be stored in a sterile vial as a solution, suspension, gel, emulsion, solid, or dehydrated or lyophilized powder. Such compositions may be stored either in a ready to use form, a lyophilized form requiring reconstitution prior to use, a liquid form requiring dilution prior to use, or other acceptable form. In some embodiments, a pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus may be used to deliver peptides and the other agents described herein, including implants (e.g., implantable pumps) and catheter systems, both of which are known to the skilled artisan. Depot injections, which are generally administered subcutaneously or intramuscularly, may also be utilized to release peptides and/or other agents described herein over a defined period of time. Depot injections are usually either solid- or oil-based and generally comprise at least one of the formulation components set forth herein. The skilled artisan is familiar with possible formulations and uses of depot injections.

A pharmaceutical composition can be formulated to be compatible with its intended route of administration. Thus, pharmaceutical compositions include carriers, diluents, or excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical).

Pharmaceutical compositions may be in the form of a sterile injectable aqueous or oleagenous suspension. This suspension may be formulated using suitable dispersing or wetting agents and suspending agents disclosed herein or known to the skilled artisan. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

Pharmaceutical compositions may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions. Such compositions may contain one or more agents such as sweetening agents, flavoring agents, coloring agents and preserving agents in order to provide pharmaceutically elegant and palatable preparations. Tablets containing a peptide provided herein may be in admixture with non-toxic pharmaceutically acceptable excipients suitable for the manufacture of tablets. These excipients include, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

Tablets, capsules and the like suitable for oral administration may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, carbohydrates, polyamine acids, hydrogel, polyvinyl pyrrolidone, polyanhydrides, polyglycolic acid, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, non encapsulated nanoparticles, microspheres, microbeads, and lipid-based systems (e.g., N-fatty acyl groups such as N-lauroyl, N-oleoyl, fatty amines such as dodecyl amine, oleoyl amine, etc., see U.S. Pat. No. 6,638,513), including oil-in-water emulsions, micelles, mixed micelles, and liposomes. Methods of preparing liposomes are described in, for example, U.S. Pat. Nos. 4,235,871, 4,501,728, and 4,837,028. Methods for the preparation of encapsulated-free constrolled release using nanoparticles are described, for example, in Pakulska et al., Science Advances 2(5): e1600519 (2016). Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions provided herein may also be in the form of oil-in-water emulsions. The oily phase may be a vegetable oil, for example olive oil or arachis oil, or a mineral oil, for example, liquid paraffin, or mixtures of these. Suitable emulsifying agents may be naturally-occurring gums, for example, gum acacia or gum tragacanth; naturally-occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

Also provided herein are peptides and/or one or more additional agents described herein in the form of suppositories for rectal administration. The suppositories can be prepared by mixing a peptide and/or one or more additional agents described herein with a suitable non-irritating excipient which is solid at ordinary temperatures but liquid at the rectal temperature and will therefore melt in the rectum to release the drug. Such materials include, but are not limited to, cocoa butter and polyethylene glycols.

### Kits

Also provided herein are kits including, but not limited to, peptide sequences provided herein and/or one or more additional agents for the treatment or prevention of a gastrointestinal motility-related disorder, or a composition comprising the foregoing, and one or more pharmaceutically acceptable or physiologically acceptable diluents, carriers or excipients, optionally in further combination with one or more therapeutic agents distinct from those described above, compositions and pharmaceutical compositions thereof, packaged into suitable packaging material. A kit may include a label or packaging insert including a description of the components or instructions for use in vitro, in vivo, or ex vivo, of the components therein. Exemplary instructions include instructions for treatment and/or prevention of the gastrointestinal motility-related disorder.

Also provided herein are kits including, but not limited to, peptide sequences provided herein and/or one or more additional agents for the treatment or prevention of constipation, or a composition comprising the foregoing, and one or more pharmaceutically acceptable or physiologically acceptable diluents, carriers or excipients, optionally in further combination with one or more therapeutic agents distinct from those described above, compositions and pharmaceutical compositions thereof, packaged into suitable packaging material. A kit may include a label or packaging insert including a description of the components or instructions for use in vitro, in vivo, or ex vivo, of the components therein. Exemplary instructions include instructions for treatment and/or prevention of constipation or for stimulating bowel function etc.

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampule, tube or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RANI and ROM or hybrids of these such as magnetic/optical storage media, FLASH media or memory type cards.

Labels or inserts can include, among other things, identifying information of one or more components therein, dosing parameters, and/or information on the clinical pharmacology of the active ingredient(s), including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimens set forth herein. Exemplary instructions include instructions for treatment or use of a peptide sequence as set forth herein and/or the use of an additional agent or treatment modality useful in treating or preventing a gastrointestinal motility-related disorder. Exemplary instructions include instructions for treatment or use of a peptide sequence as set forth herein and/or the use of an additional agent or treatment modality useful in treating or preventing constipation. Kits provided herein therefore can additionally include labels or instructions for practicing any of the methods and uses provided herein, including treatment methods and uses.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse effects could also occur when the subject has, will be, or is currently taking one or more other medications that may be incompatible with the composition, or the subject has, will be, or is currently undergoing another treatment protocol or therapeutic regimen which would be incompatible with the composition and, therefore, instructions could include information regarding such incompatibilities.

Kits provided herein can additionally include other components. Each component of the kit can be enclosed within an individual container and all of the various containers can be within a single package. In certain embodiments, kits are designed for cold storage. Kits provided herein can further be designed to contain peptide sequences provided herein, or that contain nucleic acids encoding peptide sequences. Kits provided herein can also be designed to contain, either separately or in combination with the peptide sequences provided herein, one or more additional agents useful in the treatment or prevention of a gastrointestinal motility-related disorder. Any cells in the kit can be maintained under appropriate storage conditions until ready to use.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All applications, publications, patents and other references, GenBank citations and ATCC citations cited herein are incorporated by reference in their entirety. In case of conflict, the specification, including definitions, will control. As used herein, the singular forms “a,” “and,” and “the” include plural referents unless the context clearly indicates otherwise. Thus, for example, reference to “a peptide sequence” or a “treatment,” includes a plurality of such sequences, treatments, and so forth.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges, unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1000, 1000-2500, 2500-5000, 5000-25,000, or 5000-50,000 includes any numerical value or range within or encompassing such values, e.g., 25, 26, 27, 28, 29 . . . 250, 251, 252, 253, 254 . . . 500, 501, 502, 503, 504 . . . , etc.

A series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### EXPERIMENTAL

The following descriptions are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; pl or pL=picoliter(s); dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; TIW=three times a week; QM=monthly; HPLC=high performance liquid chromatography; BW=body weight; U=unit; ns=not statistically significant; PBS=phosphate-buffered saline; PCR=polymerase chain reaction; NHS=N-Hydroxysuccinimide; HSA=human serum albumin; BSA=bovine serum albumin; DMEM=Dulbeco's Modification of Eagle's Medium; GC=genome copy; EDTA=ethylenediaminetetraacetic acid.

### Materials and Methods

The following general materials and methods can be used.

Standard Molecular Biology Techniques.

Standard methods in molecular biology are described in the scientific literature (see, e.g., Sambrook and Russell (2001) Molecular Cloning, 3^{rd} ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

Production, purification, and fragmentation of polyclonal and monoclonal antibodies are described (e.g., Harlow and Lane (1999) Using Antibodies, Cold Spring Harbor Laboratory Press, Cold Spring Harbor, N.Y.); standard techniques for characterizing ligand/receptor interactions are available (see, e.g., Coligan et al. (2001) Current Protocols in Immunology, Vol. 4, John Wiley, Inc., NY); methods for flow cytometry, including fluorescence-activated cell sorting (FACS), are available (see, e.g., Shapiro (2003) Practical Flow Cytometry, John Wiley and Sons, Hoboken, N.J.); and fluorescent reagents suitable for modifying nucleic acids, including nucleic acid primers and probes, polypeptides, and antibodies, for use, for example, as diagnostic reagents, are available (Molecular Probes (2003) Catalogue, Molecular Probes, Inc., Eugene, Oreg.; Sigma-Aldrich (2003) Catalogue, St. Louis, Mo.).

Software.

Software packages and databases for determining, e.g., antigenic fragments, leader sequences, protein folding, functional domains, glycosylation sites, and sequence alignments, are available (see, e.g., GCG Wisconsin Package™ (Accelrys, Inc., San Diego, Calif.); and DeCypher™ (TimeLogic Corp., Crystal Bay, Nev.).

Animals.

Mice can be purchased from The Jackson Laboratory (Bar Harbor, Me.) and used in various models, assays and the like familiar to the skilled artisan. By way of example, db/db mice (The Jackson Laboratory) can be kept in accordance with welfare guidelines under controlled light (12 hr light and 12 hr dark cycle, dark 6:30 pm-6:30 am), temperature (22±4° C.) and humidity (50%±20%) conditions. Mice can have free access to water (autoclaved distilled water) and can be fed ad libitum on a commercial diet (Harlan Laboratories, Indianapolis, Ind., Irradiated 2018 Teklad Global 18% Protein Rodent Diet) containing 17 kcal % fat, 23 kcal % protein and 60 kcal % carbohydrate. All animal studies can be approved by the NGM Institutional Animal Care and Use Committee.

DNA and Amino Acid Sequences.

cDNA of ORF encoding human FGF19 (Homo sapiens FGF19, GenBank Accession No. NM_005117.2) and protein sequence encoded by the cDNA (GenBank Accession No. NP_005108.1) can be used herein.

PCR.

FGF19 ORF can be amplified with polymerase chain reaction (PCR) using recombinant DNA (cDNA) prepared from human small intestinal tissue. PCR reagent kits with Phusion® high-fidelity DNA polymerase can be purchased from New England BioLabs (F-530L, Ipswich, Mass.). The following primers can be used:

forward PCR primer:
(SEQ ID NO: 136)
5′ CCGACTAGTCACCatgcggagcgggtgtgtgg
and
reverse PCR primer:
(SEQ ID NO: 137)
5′ ATAAGAATGCGGCCGCTTACTTCTCAAAGCTGGGACTCCTC.

Amplified DNA fragment can be digested with restriction enzymes Spe I and Not I (the restriction sites are frequently not included in the 5′ or 3′ PCR primers, respectively) and then ligated with AAV transgene vectors that have been digested with the same restriction enzymes. The vector that can be used for expression can contain a selectable marker and an expression cassette comprising a strong eukaryotic promoter 5′ of a site for insertion of the cloned coding sequence, followed by a 3′ untranslated region and a bovine growth hormone polyadenylation tail. The expression construct can also be flanked by internal terminal repeats at the 5′ and 3′ ends.

Production and Purification of AAV.

AAV293 cells (which can be obtained from Agilent Technologies, Santa Clara, Calif.) can be cultured in Dulbeco's Modification of Eagle's Medium (DMEM, Mediatech, Inc. Manassas, Va.) supplemented with 10% fetal bovine serum and 1× antibiotic-antimycotic solution (Mediatech, Inc. Manassas, Va.). The cells can be plated at 50% density on day 1 in 150 mm cell culture plates and can be transfected on day 2, using calcium phosphate precipitation method with the following 3 plasmids (20 μg/plate of each): AAV transgene plasmid, pHelper™ plasmids (Agilent Technologies) and AAV2/9 plasmid (Gao et al., J. Virol. 78:6381 (2004)). Forty-eight (48) hours after transfection, the cells can be scraped off the plates, pelleted by centrifugation at 3000×g and resuspended in buffer containing 20 mM Tris pH 8.5, 100 mM NaCl and 1 mM MgCl₂. The suspension can be frozen in an alcohol dry ice bath and then thawed in a 37° C. water bath. The freeze and thaw cycles can be repeated three times; Benzonase® (Sigma-aldrich, St. Louis, Mo.) can be added to 50 units/ml; deoxycholate can be added to a final concentration of 0.25%. After incubation at 37° C. for 30 min, cell debris can be pelleted by centrifugation at 5000×g for 20 min. Viral particles in the supernatant can be purified using a gradient comparable to discontinued iodixanal (Sigma-aldrich, St. Louis, Mo.) gradient as previously described (Zolotukhin S. et al (1999) Gene Ther. 6:973). The viral stock can be concentrated using Vivaspin® 20 (MW cutoff 100,000 Dalton, Sartorius Stedim Biotech, Aubagne, France) and resuspended in phosphate-buffered saline (PBS) with 10% glycerol and stored at −80° C. To determine the viral genome copy number, 2 μl of viral stock can be incubated in 6 μl of solution containing 50 units/ml Benzonase®, 50 mM Tris-HCl pH 7.5, 10 mM MgCl₂ and 10 mM CaCl₂) at 37° C. for 30 minutes.

Afterwards, 15 μl of the solution containing 2 mg/ml of Proteinase K, 0.5% SDS and 25 mM EDTA can be added and the mixture can be incubated for an additional 20 min at 55° C. to release viral DNA. Viral DNA can be cleaned with mini DNeasy® Kit (Qiagen, Valencia, Calif.) and eluted with 40 μl of water. Viral genome copy (GC) can be determined by using quantitative PCR. Viral stock can be diluted with PBS to desirable GC/ml, and viral working solution (200 μl) can be delivered into mice via tail vein injection.

Scintigraphic Transit Assay: Gastric Emptying, Colonic Transit and Ascending Colon.

Gastric and colonic transit measurements were performed after an overnight fast using a well-validated scintigraphy method. To evaluate colonic transit, ¹¹¹In adsorbed on activated charcoal particles was delivered to the colon by a methacrylate-coated capsule. All participants ingested ingested a 99mTc egg meal (320 kcal, 30% fat; two scrambled eggs with one slice of whole wheat bread and one glass of skim milk) and the capsule containing ¹¹¹In charcoal. When the capsule had been demonstrated to have emptied from the stomach, all participants received a standard meal. A variable region of interest program was used to measure transit. Abdominal images were obtained. Abdominal images were obtained at standard times including scans every 15 minutes in the first 4 hours for estimation of gastric emptying, as well as at 6, 8, 24, 32 and 48 hours for detailed estimation of colonic transit. Endpoints measured included gastric emptying half-life time (“GE t_{1/2}”); colonic geometric centre (“GC”) at 24 h; and ascending colon emptying (“AC t_{1/2}”).

Measuring Stool Frequency and Consistency and Ease of Passage.

Subjects recorded stool frequency, consistency, sense of evacuation, and ease of stool passage on daily bowel diaries during the run-in and treatment periods. The stool diary was dispensed at visit 1 (screening) and at visit 5 (randomization). Stool frequency is represented as number of Bowel Movement per week (“# BM/week”). Ease of Passage was evaluated on an adjectival scale (a 7-point adjectival scale ranging from manual disimpaction=1 to incontinence=7). Stool consistency was measured using the Bristol Stool Form Scale (“BSFS”).

Assay to Measure Fecal Fat Excretion:

Quantitative fecal fat test was used to measure amount of fat. After consuming a 100-g fat diet for 48 hours, participants collected all stool samples during the next 48 hours while ingesting the same diet. The samples were kept cold or frozen and aliquots of the 48-hour stool collection were analyzed using the van de Kamer method for fat.

Assay to Measure Fecal Bile Acids Excretion and Proportion of Primary Bile Acids

Both the total amount of fecal bile acids (data represented as “48 h fecal BA, umol LSM” or “fecal BA mmol/48 h”) and the percentage of the primary bile acids, CA and CDCA, as a percent of the total fecal bile acids (data represented as “% CA+CDCA LSM”) were measured. After consuming a 100-g fat diet for 48 hours, participants collected all stool samples during the next 48 hours while ingesting the same diet. The samples were kept cold or frozen and aliquots of the 48-hour stool collection were analyzed using the HPLC/tandem mass spectrometry for bile acids (Wong et al., Clin Gastroenterol Hepatol 10:1009-1015, e3 (2012)).

Assay to Measure Hepatic Bile Acid Synthesis:

Fasting morning serum C4 was measured by liquid chromatography-tandem mass spectrometry (Camilleri et al., Neurogastroenterol Motil. 21:734-e43(2009)) at baseline and during administration of study drug for estimation of hepatic bile acid synthesis.

Genotyping:

Genomic DNA was isolated from peripheral blood leukocytes as described in previous studies (Qiagen Kit, Qiagen Corp., Germantown, Md., USA). Variations in the genes of interest (KLB rs17618244, FGFR4 rs351855 and TGR5 rs11554825) were assayed by TaqMan® SNP Genotyping Assays (Applied Biosystems; Foster City, Calif., USA) according to the manufacturer's instructions, using 10-20 ng DNA using previously described techniques (See Wong et al., Gastroenterology 140:1934-1942 (2011) and Camilleri et al., Am J Physiol 307:G508-G516 (2014)). Following polymerase chain reaction (PCR) amplification, end reactions were read on the ABI 7500 Fast Real-Time PCR System using Sequence Detection Software version 1.3.1 (Applied Biosystems, Thermo Fisher Scientific, Waltham, Mass., USA).

### Example 1

This example describes a Phase 1B, two-dose, placebo controlled, randomized double-blind study clinical study to evaluate the effects of M70 on colonic transit, stool frequency and consistency, fecal fat and bile acids (serum and fecal) in patients with functional constipation (“FC”), and shows the role of M70 in treating FC in human patients.

Study Design and Statistical Analysis:

M70 is a non-tumorigenic engineered variant of human fibroblast growth factor 19 (FGF19), which has shown biologic activity in patients with diabetes and liver diseases (PBC, PSC and NASH). In patients with FC (Rome III criteria) and baseline Colonic Transit (“CT”) at 24 h<3.0, we conducted a 2-dose (1 and 6 mg SQ daily) M70 parallel group, placebo-controlled randomized double-blind study with treatment lasting 14 days and evaluated the effects on CT (primary endpoint), stool frequency and consistency (Bristol stool form scale (“BSFS”)), fecal fat and total and individual fecal bile acids (“BA”). Randomization of the study was performed in fixed block sizes and the patients, investigators and statistician were blinded to group assignments.

Participants:

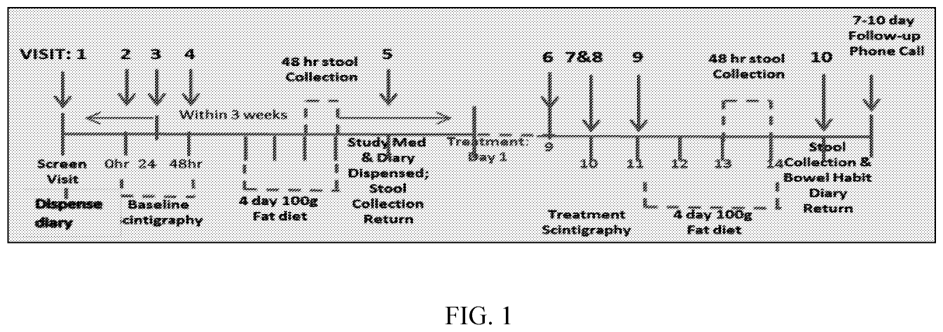

Thirty-one subjects with functional constipation were enrolled in the study. The main eligibility criteria were females who were not pregnant or not nursing, 18 to 65 years of age, diagnosed with functional constipation by Rome III criteria and baseline colonic transit geometric center of <3.0 at 24 hours, no evidence of gastrointestinal diseases and not on medications that would affect the gastrointestinal system. Patients completed ten visits during the study, including a screening medical and physical examination, fasting screening laboratory tests including a comprehensive metabolic profile, a 12-lead electrocardiogram, and completion of a bowel disease questionnaire. Eligible patients underwent baseline colonic transit at 24 hours and baseline 48-hour stool fat, and bile acid measurement after eating a high fat (100 g) diet for 4 days with stool collection in the final 2 days of the high fat diet. Patients received a study drug kit and diary for recording bowel functions and date/time of study drug administration. The subjects were divided into three groups, receiving either placebo, 1 mg of M70, or 6 mg of M70. M 70 was provided as a sterile solution of identical volume (0.3 ml) in pre-filled syringes delivering doses of 1 mg and 6 mg, as well as placebo (0.3 ml), and was self-administered subcutaneously by patients. FIG. 1 illustrates the study timeline.

The following methods were used during baseline off treatment and during the treatment period: scintigraphic gastrointestinal (“GI”) transit and CT, total 48 h fecal fat and BA excretion, proportion of primary bile acids (“main 1° bile acids”) (cholic acid (“CA”) and chenodeoxycholic acid (“CDCA”)), fasting serum C4, stool frequency and consistency (BSFS: 1-7) and ease of passage (scale 1-7). An exploratory pharmacogenetics association study was performed to assess the interaction of SNPs in genes of KLB, FGFR4 and TGR5 (GBPAR1) with the effects of M70 on colonic transit.

Study Power and Analysis:

Based on the observed data in the placebo group, 10 subjects per group were expected to provide 80% power to detect ˜35% changes in the primary endpoint of the study, overall CT at 24 and 48 h (Table 2). Data are expressed as the mean±standard error of the mean (“SEM”). Statistical analysis included overall ANCOVA at α=0.025 (baseline measurement as covariate where available), with 3 pairwise comparisons among the three groups at α=0.008 to correct for multiple comparisons. All randomized subjects were included in the intent to treat analysis. All baseline characteristics and demographic data were summarized using the intent to treat analysis. The intent to treat analysis was based on randomized treatment, if this differed from actual treatment received.

TABLE 2
Intent to Treat analysis. Effect sizes detectable with 80%
power based on a two-sample t-test using a two-sided
alpha level of 0.025, with 10 subjects per group.

| | | | | Effect size | Effect size |
|---|---|---|---|---|---|
| | | | | detectable | detectable |
| | | | | with 80% power | with 80% power |
| | | | | (α = 0.025) as | (α = 0.025) as |
| Quantitative | | | CV | percentage | percentage |
| trait | Mean | SD | (%) | of listed mean | of listed one SD |
| Colon GC 24 h | 2.70 | 0.83 | 31 | 37% | 146% |
| Colon GC 48 h | 4.47 | 0.61 | 14 | 30% | Na |
| Serum C4, | 20.6 | 24.2 | 117 | 92% | 149% |
| ng/mL | | | | | |
| Total fecal bile | 303 | 288 | 95 | 74% | 77% |
| acids, | | | | | |
| μmol/48 h | | | | | |

“Na” = not available; “SD” = standard deviation; “CV” = coefficient of variation

Results:

Table 3 shows demographics, baseline and on treatment results for colonic transit, bowel function, fecal weight, fat and bile acid measurements.

Participants and Baseline Data:

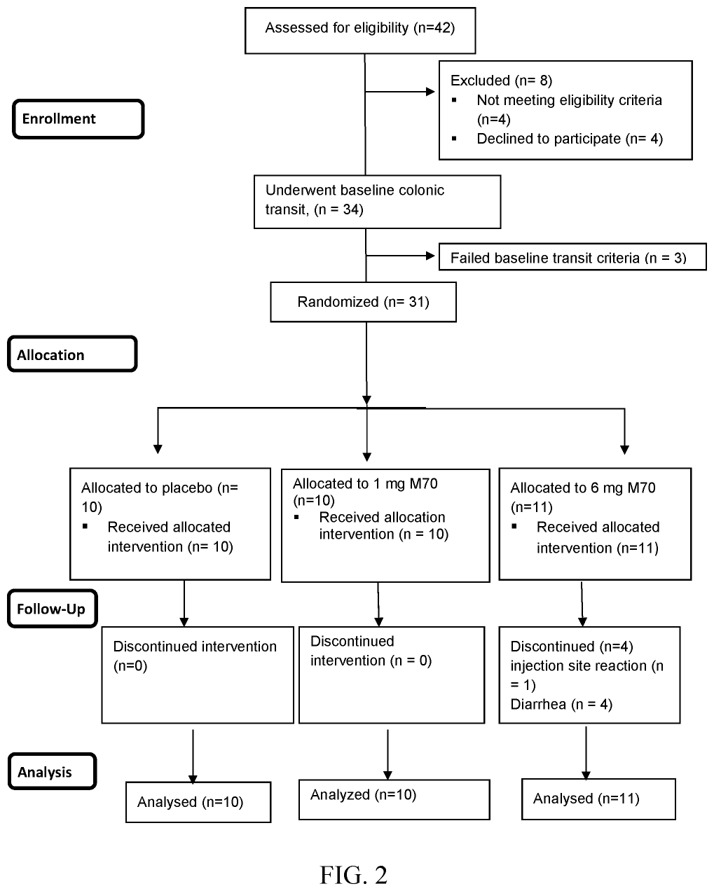

Forty-two female subjects were assessed for eligibility in the study from Dec. 22, 2015 through Dec. 5, 2016 (FIG. 2). Four subjects declined to participate, four did not meet study criteria, and three had colonic transit GC>3.0 at 24 hours; thus, thirty-one subjects were randomized to the study. Ten subjects received placebo, ten received 1 mg of M70, and eleven received 6 mg of M70. Four patients withdrew from the study; three because of diarrhea and one because of injection site reaction, but they were included in the intent to treat analysis. There was no statistically significant difference between the baseline data of all three groups (Table 3). Baseline colonic transit geometric center was 1.91±1.0 which is similar to the 2.0±1.0 (SEM) that has been reported in the past in patients with functional constipation (Manabe et al., Neurogastroenterol Motil 22:293-e82 (2010)).

Effects of M70 on Gastric Emptying and Colonic Transit:

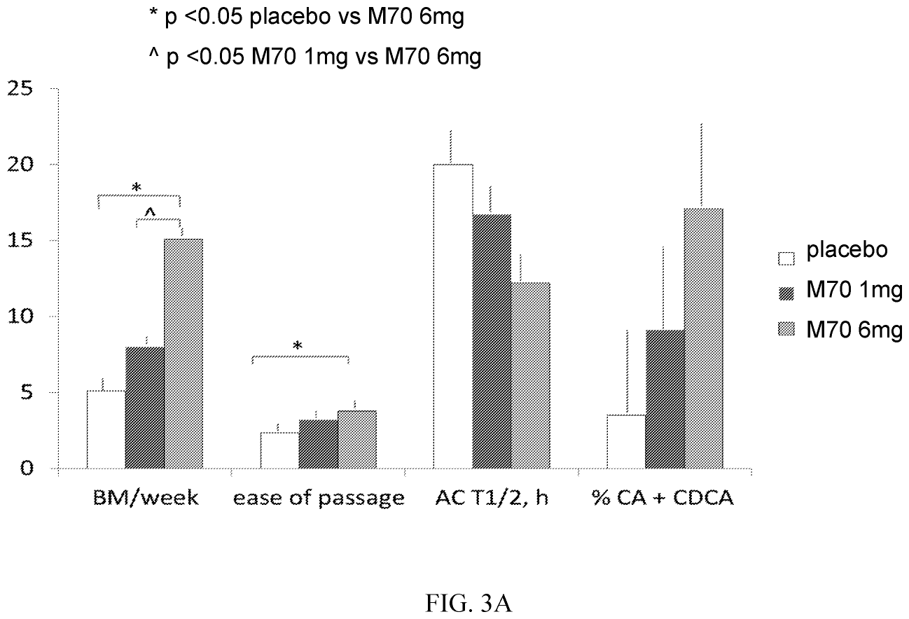

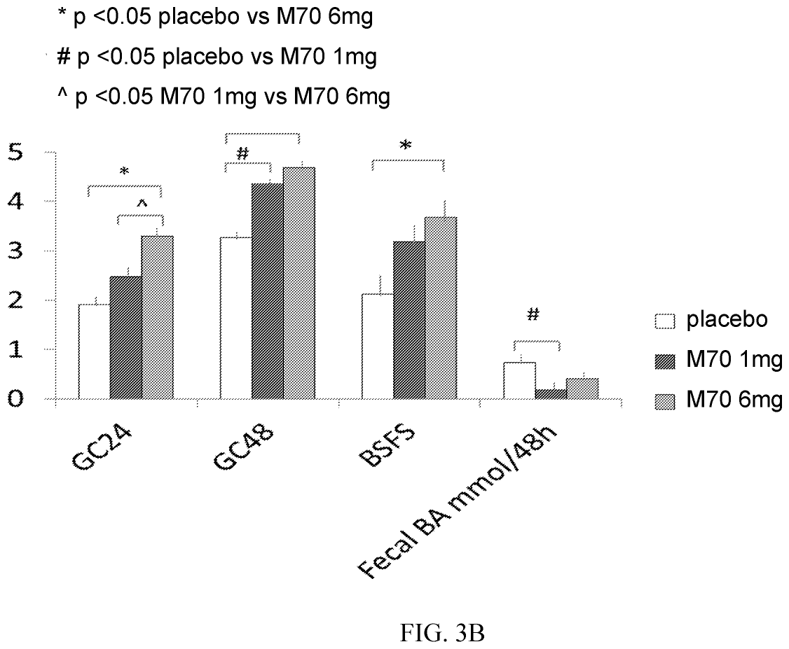

M70 significantly accelerated gastric emptying and overall colonic transit at 24 and 48 hours compared to placebo (Table 3). Both M70 dose groups had acceleration of gastric emptying compared to placebo. The effect of M70 on colonic transit at 24 hours was dose dependent; there was more significant acceleration with M70 6 mg, compared to the M70 1 mg group. At 48 hours, there was also significant acceleration with M70, 1 mg and 6 mg, compared to placebo (FIG. 3B). Some overall effect of M70 (for both the 1 mg and the 6 mg doses) on ascending colon emptying was also observed.

Effects of M70 on Bowel Function and Stool Fat:

Table 3 shows treatment effects, estimated as least square means, that is, adjusting for baseline values, on bowel function and stool fat. Participants who received M70 6 mg had significantly more bowel movements per week compared to participants who received M70 1 mg or placebo, with average bowel movements of 15, 8, and 5 per week, respectively, in the three groups (FIG. 3A). Stool form was significantly softer and easier to pass for participants who received M70 6 mg compared to placebo. Stool weight and fecal fat excretion were normal and similar in the three groups.

Effects of M70 on Bile Acids and Bile Synthesis:

Bile acid excretion decreased and the proportion of primary bile acids increased in patients who received M70 compared to placebo (Table 3). The decrease in bile acid excretion was significant for M70 1 mg compared to placebo. Bile acid synthesis, as measured by C4, similarly decreased in patients that received M70 compared to placebo (Table 3).

Exploratory Analysis of GPBAR1, FGFR4, KLB Genotypes and Effects on Bile Acid Excretion, Colonic Transit, Gastric Emptying and Bowel Function by Group:

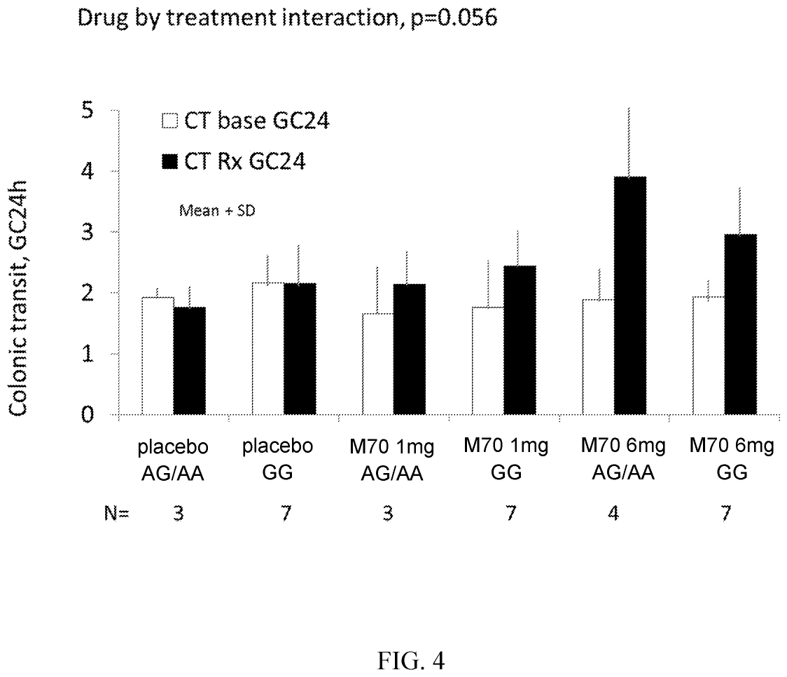

A greater acceleration of colonic transit at 24 hours was detected in participants with the Klothoβ (KLB) rs17618244 A (minor) allele (KLB Gln728) compared to KLB major (G) allele (KLB Arg728) in response to M70 6 mg, supporting an interaction between this genotype and response to colonic transit (FIG. 4). There were no significant interactions detected between treatment and FGFR4 rs351855 and GBPAR1 rs11554825 genotypes.

Tolerability and Safety:

Participants who received M70 treatments reported more side effects, including headache, increased appetite, and injection site reaction, compared to the placebo group. However, increased appetite was the only adverse effect that was statistically significant.

Hence, M70 significantly (bolded p values in table) accelerated gastric emptying (“GE”), ascending colon (“AC”) and overall CT (values shown are least square means [LSM] adjusted for baseline values), increased weekly BM frequency and loosened stool consistency. There was no steatorrhea, but there was reduced fecal total BA excretion. (Table 3; FIGS. 3A and 3B).

TABLE 3
Demographics and baseline off treatment results for colonic transit, bowel function,
fecal weight, fat measurements and bile measurements in patients with functional constipation.

| | | | | P | 1 mg vs | 6 mg vs | 1 mg vs |
|---|---|---|---|---|---|---|---|
| Data mean ± SEM | placebo | M70 1 mg | M70 6 mg | ANOVA | placebo | placebo | 6 mg |
| N | 10 | 10 | 11 | | | | |
| Age, y | 46.5 ± 3.15 | 44.3 ± 3.04 | 40.4 ± 3.83 | Ns | | | |
| BMI, kg/m² | 26.9 ± 1.2 | 26.2 ± 1.3 | 26.1 ± 1.3 | Ns | | | |

Baseline data

| | | | | |
|---|---|---|---|---|
| CT GC24 | 2.09 ± 0.14 | 1.73 ± 0.23 | 1.92 ± 0.11 | Ns |
| CT GC48 | 3.46 ± 0.28 | 2.96 ± 0.25 | 3.13 ± 0.31 | Ns |
| Fecal fat, g/day | 2.8 ± 0.8 | 3.9 ± 1.0 | 4.0 ± 0.9 | Ns |
| 48 h stool weight, g | 143 ± 47 | 160 ± 40 | 148 ± 41 | Ns |
| 48 h fecal BA, μmol | 385 ± 87 | 613 ± 175 | 468 ± 203 | Ns |
| % CA + CDCA | 6.0 ± 4.1 | 1.1 ± 0.3 | 3.9 ± 1.8 | Ns |
| **Fasting serum | 15.5 (9.7-35.0) | 7.8 (4.4-16.0) | 7.6 (4.8-22.0) | |
| C4, ng/mL | | | | |

On treatment data

| | | | | | | | |
|---|---|---|---|---|---|---|---|
| GE T_{1/2} (min) | 132.4 ± 7.4 | 97.8 ± 7.2 | 94.5 ± 8.1 | 0.002 | 0.003 | 0.001 | 0.850 |
| CT GC24 LSM | 2.03 ± 0.16 | 2.35 ± 0.18 | 3.30 ± 0.29 | <0.001 | 0.07 | <0.001 | 0.007 |
| CT GC48 LSM | 3.36 ± 0.29 | 4.29 ± 0.22 | 4.66 ± 0.13 | <0.001 | 0.001 | <0.001 | 0.290 |
| AC T_{1/2}, h | 20.0 ± 2.9 | 16.7 ± 1.8 | 12.2 ± 1.6 | 0.049 | 0.300 | 0.015 | 0.148 |
| BSFS/Stool Form | 2.12 ± 0.36 | 3.19 ± 0.34 | 3.68 ± 0.32 | 0.009 | 0.036 | 0.003 | 0.308 |
| # BM/week | 5.1 ± 0.5 | 8.0 ± 0.7 | 15.1 ± 2.3 | <0.001 | 0.178 | <0.001 | 0.002 |
| Ease of passage | 2.35 ± 0.27 | 3.19 ± 0.19 | 3.79 ± 0.24 | <0.001 | 0.02 | <0.001 | 0.079 |
| 48 h stool weight, g | 259 ± 66 | 191 ± 43 | 270 ± 45 | 0.27 | 0.19 | 0.90 | 0.14 |
| Fecal fat, g/day | 5.4 ± 2.1 | 5.4 ± 0.9 | 9.2 ± 2.3 | 0.65 | 0.37 | 0.78 | 0.53 |
| 48 h fecal BA, μmol | 751 ± 176 | 190 ± 34 | 403 ± 150 | 0.003 | <0.001 | 0.014 | 0.19 |
| LSM | | | | | | | |
| % CA ± CDCA | 3.9 ± 1.4 | 7.9 ± 6.1 | 17.9 ± 6.9 | 0.096 | 0.71 | 0.098 | 0.046 |
| LSM | | | | | | | |
| Fasting C4 ng/mL | 27.7 ± 7.9 | 9.5 ± 4.3 | 2.6 ± 1.9 | 0.89 | 0.98 | 0.72 | 0.65 |
| Fasting C4, change | −5.9 ± 5.2 | 2.9 ± 4.0 | 8.3 ± 2.3 | 0.056 | | | |
| baseline-post, | | | | | | | |
| ng/mL | | | | | | | |

| | | | |
|---|---|---|---|
| Fasting C4 compared to baseline | 0.143 | 0.468 | 0.049 |

Bolded values are statistically significant;
GE T_{1/2} available from 10 in M70 6 mg group;
“Ns” = not significant;
“LSM” = least square means;
**Median and IQR presented as data were not normally distributed;
48 h stool weight and fecal fat available from 9 people in placebo and 10 in M70 6 mg groups.

As shown, M70 significantly accelerated gastric emptying and colonic transit, altered bowel function. Thus, M70 represents a novel and effective therapeutic to treat gastroparesis and constipation.

The effect of M70 appears to be related to increased colonic motility rather than increased small bowel or colonic secretion, which is supported by the fact that stool weight was similar in placebo and M70 groups, and the relative increase in stool consistency was low compared to the increase in the numbers of stools in patients who received M70.

As supported by this study which demonstrated a drug-by-genotype interaction between KLB variant and the effect of M70 on colonic transit, M70 can stimulate excitatory neural control of colonic motility.

Previous work in IBS-D has demonstrated that the protein obtained from the major allele is less stable than that produced by the minor allele, with less feedback inhibition of bile acid synthesis and faster colonic transit in a proportion of IBS-D patients with the major phenotype. Wong B S, et al., Gastroenterology 2011; 140:1934-1942. In this study, M70 decreased bile acid excretion by decreasing bile acid synthesis, which would have been expected to result in further delayed transit in participants with the minor allele and stable Klothoβ protein. However, this was the opposite of what we observed, evidencing an entirely different mechanism for the action of M70 on colonic transit.

For example, M70 in the systemic circulation can act on Klothoβ in nerve cells to increase colonic transit. Studies in mice have shown that Klothoβ deficient mice age prematurely, and this may be linked to decrease in cholinergic signaling through the M1 muscarinic cholinergic receptor. Park S J, et al., Neuropsychopharmacology 2013; 38:1426-1437. FGF15 (murine ortholog of human FGF19) has been shown to regulate neural tube formation in mice. Yang S L, et al. Dev Biol 2015; 408:140-150; Fischer T, et al., Dev Biol 2011; 350:496-510. Thus, Klothoβ controls neural tube development, and Klotho mutant mice age prematurely and have been found to have lower cholinergic expression. This support that M70's action on colonic transit is not related to the effects of the FGF19 analog through the FGFR4 receptor in the liver, but at other peripheral sites, such as nerve cells that modulate colonic transit.

Previous trials on bile acids (Rao A S, et al., Gastroenterology 2010; 139:1549-1558), colesevelam (Odunsi-Shiyanbade S T, et al., Clin Gastroenterol Hepatol 2010; 8:159-165) and ileal bile acid transporter inhibitor (IBAT) (Wong B S, et al. Am J Gastroenterol 2011; 106:2154-2164) have shown a modest decrease in gastric emptying with an inverse relationship between gastric emptying and colonic transit. This action is presumed to be from bile acid binding of G protein coupled receptor, TGR5 (Hansen M, et al. Diabetes Obes Metab 2016; 18:571-580), on enteroendocrine cells with resultant secretion of glucagon like peptide 1 (GLP-1). However, surprisingly, in this study of M70, gastric and colonic transit were both accelerated, evidencing this was not an effect mediated by bile acids, but rather the action of M70 on nerve cells with neuromuscular stimulation in the gastrointestinal tract.

### SEQUENCE LISTING

The present specification is being filed with a computer readable form (CRF) copy of the Sequence Listing. The CRF entitled 13370-072-228_SEQ_LISTING.txt, which was created on Mar. 24, 2018 and is 256,748 bytes in size, is incorporated herein by reference in its entirety.

1-135. (canceled)

136. A method of treating or preventing a gastrointestinal motility-related disorder in a subject, comprising administering to the subject a therapeutically effective amount of a chimeric peptide sequence, comprising:
i) an N-terminal region comprising at least seven amino acid residues, the N-terminal region having a first amino acid position and a last amino acid position, wherein the N-terminal region comprises DSSPL (SEQ ID NO:121) or DASPH (SEQ ID NO:122), and
ii) a C-terminal region comprising a portion of SEQ ID NO:99 (FGF19), the C-terminal region having a first amino acid position and a last amino acid position, wherein the C-terminal region comprises amino acid residues 16-29 of SEQ ID NO:99 (FGF19), WGDPIRLRHLYTSG (SEQ ID NO:169), wherein the W residue corresponds to the first amino acid position of the C-terminal region;
thereby treating or preventing the gastrointestinal motility-related disorder in the subject.

137. A method of treating or preventing a gastrointestinal motility-related disorder in a subject, comprising administering to the subject a therapeutically effective amount of a chimeric peptide sequence, comprising:
i) an N-terminal region comprising a portion of SEQ ID NO:100 (FGF21), the N-terminal region having a first amino acid position and a last amino acid position, wherein the N-terminal region comprises amino acid residues GQV, and wherein the V residue corresponds to the last amino acid position of the N-terminal region, and
ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region;
thereby treating or preventing the gastrointestinal motility-related disorder in the subject.

138. The method of claim 137, wherein the N-terminal region comprises at least 5 contiguous amino acids of SEQ ID NO:100 (FGF21) including the amino acid residues GQV.

139. The method of claim 136, wherein treating or preventing the gastrointestinal motility-related disorder comprises stimulating bowel function in the subject.

140. The method of claim 137, wherein treating or preventing the gastrointestinal motility-related disorder comprises stimulating bowel function in the subject.

141. The method of claim 136, wherein treating or preventing the gastrointestinal motility-related disorder comprises treating or preventing constipation in the subject.

142. The method of claim 137, wherein treating or preventing the gastrointestinal motility-related disorder comprises treating or preventing constipation in the subject.

143. The method of claim 136, wherein the peptide sequence has amino-terminal amino acids 1-16 of SEQ ID NO:100 (FGF21) fused to carboxy-terminal amino acids 21-194 of SEQ ID NO:99 (FGF19), or wherein the peptide sequence has amino-terminal amino acids 1-147 of SEQ ID NO:99 (FGF19) fused to carboxy-terminal amino acids 147-181 of SEQ ID NO:100 (FGF21) (M41), or wherein the peptide sequence has amino-terminal amino acids 1-20 of SEQ ID NO:99 (FGF19) fused to carboxy-terminal amino acids 17-181 of SEQ ID NO:100 (FGF21) (M44), or wherein the peptide sequence has amino-terminal amino acids 1-146 of SEQ ID NO:100 (FGF21) fused to carboxy-terminal amino acids 148-194 of SEQ ID NO:99 (FGF19) (M45), or wherein the peptide sequence has amino-terminal amino acids 1-20 of SEQ ID NO:99 (FGF19) fused to internal amino acids 17-146 of SEQ ID NO:100 (FGF21) fused to carboxy-terminal amino acids 148-194 of SEQ ID NO:99 (FGF19) (M46).

144. The method of claim 136, wherein the peptide sequence comprises a substitution to one of amino acid residues 127-128 of SEQ ID NO:99 (FGF19), IRP, wherein at least one amino acid substitution is R127L or P128E.

145. The method of claim 137, wherein the peptide sequence comprises a substitution to one of amino acid residues 127-128 of SEQ ID NO:99 (FGF19), IRP, wherein at least one amino acid substitution is R127L or P128E.

146. The method of claim 136, wherein the peptide sequence comprises or consists of any sequence set forth herein as M1 to M98, M101 to M160 or M200 to M207, or SEQ ID NOs:1 to 98, 138 to 168, or 192 to 204, or any of the foregoing sequences wherein an N-terminal R residue is deleted.

147. The method of claim 136, wherein the peptide sequence maintains or increases a FGFR4 mediated activity.

148. The method of claim 137, wherein the peptide sequence maintains or increases a FGFR4 mediated activity.

149. The method of claim 136, wherein the peptide sequence is distinct from a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the FGF19 WGDPI (SEQ ID NO:170) sequence at amino acids 16-20.

150. The method of claim 136, wherein the N-terminal region comprises amino acid residues VHYG (SEQ ID NO:101), wherein the N-terminal region comprises amino acid residues DASPHVHYG (SEQ ID NO:102), or wherein the N-terminal region comprises amino acid residues DSSPLVHYG (SEQ ID NO:103).

151. The method of claim 150, wherein the G corresponds to the last position of the N-terminal region.

152. The method of claim 136, wherein the N-terminal region comprises amino acid residues DSSPLLQ (SEQ ID NO:104), and wherein the Q residue is the last amino acid position of the N-terminal region.

153. The method of claim 151, wherein the N-terminal region further comprises: RHPIP (SEQ ID NO:106), where R is the first amino acid position of the N-terminal region; or HPIP (SEQ ID NO:107), where H is the first amino acid position of the N-terminal region; or RPLAF (SEQ ID NO:108), where R is the first amino acid position of the N-terminal region; or PLAF (SEQ ID NO:109), where P is the first amino acid position of the N-terminal region; or R, where R is the first amino acid position of the N-terminal region.

154. The method of claim 136, wherein the N-terminal region comprises amino acid residues DSSPLLQFGGQV (SEQ ID NO:105), and wherein the V residue corresponds to the last position of the N-terminal region.

155. The method of claim 136, wherein amino acid residues HPIP (SEQ ID NO:107) are the first 4 amino acid residues of the N-terminal region.

156. The method of claim 137, wherein the N-terminal region comprises amino acid residues DSSPLLQFGGQV (SEQ ID NO:105), and wherein the V residue corresponds to the last position of the N-terminal region.

157. The method of claim 137, wherein amino acid residues HPIP (SEQ ID NO:107) are the first 4 amino acid residues of the N-terminal region.

158. The method of claim 136, wherein the peptide sequence at comprises or consists of any of:
HPIPDSSPLLQFGGQVRLRHLYTSG (M5-R)(amino acids 1-25
of SEQ ID NO: 160);
DSSPLLQFGGQVRLRHLYTSG (M6-R)(amino acids 2-22 of
SEQ ID NO: 6);
RPLAFSDSSPLLQFGGQVRLRHLYTSG (M7)(amino acids 1-27
of SEQ ID NO: 7);
HPIPDSSPLLQWGDPIRLRHLYTSG (M8-R)(amino acids 2-26
of SEQ ID NO: 8);
HPIPDSSPLLQFGWGDPIRLRHLYTSG (M9-R)(amino acids 2-
28 of SEQ ID NO: 9);
HPIPDSSPHVHYGWGDPIRLRHLYTSG (M10-R)(amino acids 2-
28 of SEQ ID NO: 10);
RPLAFSDAGPLLQWGDPIRLRHLYTSG (M11)(amino acids 1-27
of SEQ ID NO: 11);
RPLAFSDAGPLLQFGWGDPIRLRHLYTSG (M12)(amino acids 1-
29 of SEQ ID NO: 12);
RPLAFSDAGPLLQFGGQVRLRHLYTSG (M13)(amino acids 1-27
of SEQ ID NO: 13);
HPIPDSSPHVHYGGQVRLRHLYTSG (M14-R)(amino acids 2-26
of SEQ ID NO: 14);
RPLAFSDAGPHVHYGGQVRLRHLYTSG (M15)(amino acids 1-27
of SEQ ID NO: 15);
RPLAFSDAGPHVHWGDPIRLRHLYTSG (M16)(amino acids 1-27
of SEQ ID NO: 16);
RPLAFSDAGPHVGWGDPIRLRHLYTSG (M17)(amino acids 1-27
of SEQ ID NO: 17);
RPLAFSDAGPHYGWGDPIRLRHLYTSG (M18)(amino acids 1-27
of SEQ ID NO: 18);
RPLAFSDAGPVYGWGDPIRLRHLYTSG (M19)(amino acids 1-27
of SEQ ID NO: 19);
RPLAFSDAGPVHGWGDPIRLRHLYTSG (M20)(amino acids 1-27
of SEQ ID NO: 20);
RPLAFSDAGPVHYWGDPIRLRHLYTSG (M21)(amino acids 1-27
of SEQ ID NO: 21);
RPLAFSDAGPHVHGWGDPIRLRHLYTSG (M22)(amino acids 1-
27 of SEQ ID NO: 22);
RPLAFSDAGPHHGWGDPIRLRHLYTSG (M23)(amino acids 1-27
of SEQ ID NO: 23);
RPLAFSDAGPHHYWGDPIRLRHLYTSG (M24)(amino acids 1-27
of SEQ ID NO: 24);
RPLAFSDAGPHVYWGDPIRLRHLYTSG (M25)(amino acids 1-27
of SEQ ID NO: 25);
RPLAFSDSSPLVHWGDPIRLRHLYTSG (M26)(amino acids 1-27
of SEQ ID NO: 26);
RPLAFSDSSPHVHWGDPIRLRHLYTSG (M27)(amino acids 1-27
of SEQ ID NO: 27);
RPLAFSDAGPHVWGDPIRLRHLYTSG (M28)(amino acids 1-26
of SEQ ID NO: 28);
RPLAFSDAGPHVHYWGDPIRLRHLYTSG (M29)(amino acids 1-
28 of SEQ ID NO: 29);
RPLAFSDAGPHVHYAWGDPIRLRHLYTSG (M30)(amino acids 1-
29 of SEQ ID NO: 30);
RHPIPDSSPLLQFGAQVRLRHLYTSG (M31)(amino acids 1-26
of SEQ ID NO: 31);
RHPIPDSSPLLQFGDQVRLRHLYTSG (M32)(amino acids 1-26
of SEQ ID NO: 32);
RHPIPDSSPLLQFGPQVRLRHLYTSG (M33)(amino acids 1-26
of SEQ ID NO: 33);
RHPIPDSSPLLQFGGAVRLRHLYTSG (M34)(amino acids 1-26
of SEQ ID NO: 34);
RHPIPDSSPLLQFGGEVRLRHLYTSG (M35)(amino acids 1-26
of SEQ ID NO: 35);
RHPIPDSSPLLQFGGNVRLRHLYTSG (M36)(amino acids 1-26
of SEQ ID NO: 36);
RHPIPDSSPLLQFGGQARLRHLYTSG (M37)(amino acids 1-26
of SEQ ID NO: 37);
RHPIPDSSPLLQFGGQIRLRHLYTSG (M38)(amino acids 1-26
of SEQ ID NO: 38);
RHPIPDSSPLLQFGGQTRLRHLYTSG (M39)(amino acids 1-26
of SEQ ID NO: 39);
RHPIPDSSPLLQFGWGQPVRLRHLYTSG (M40)(amino acids 1-
28 of SEQ ID NO: 40);
DAGPHVHYGWGDPIRLRHLYTSG (M74-R)(amino acids 2-24
of SEQ ID NO: 74);
VHYGWGDPIRLRHLYTSG (M75-R)(amino acids 2-19 of SEQ
ID NO: 75);
RLRHLYTSG (M77-R)(amino acids 2-10 of SEQ ID NO:
77);
or any of the foregoing peptide sequences wherein the amino terminal R residue is deleted.

159. The method of claim 158, wherein the peptide sequence further comprises the addition of amino acid residues 30-194 of SEQ ID NO:99 (FGF19) at the C-terminus, resulting in a chimeric polypeptide.

160. The method of claim 158, wherein the peptide sequence further comprises all or a portion of a FGF19 sequence set forth as:
PHGLSSCFLRIRADGVVDCARGQSAHSLLEIKAVALRTVAIKGVHSVRYLCMGA DGKMQGLLQYSEEDCAFEEEIRPDGYNVYRSEKHRLPVSLSSAKQRQLYKNRGF LPLSHFLPMLPMVPEEPEDLRGHLESDMFSSPLETDSMDPFGLVTGLEAVRSPSFE K (SEQ ID NO:188) positioned at the C-terminus of the peptide, or wherein the amino terminal “R” residue is deleted from the peptide.

161. The method of claim 136, wherein the N-terminal region comprises any one of the following sequences: MDSSPL (SEQ ID NO:119), MSDSSPL (SEQ ID NO:120), SDSSPL (SEQ ID NO:112), MSSPL (SEQ ID NO:113), or SSPL (SEQ ID NO:114).

162. The method of claim 136, wherein the peptide sequence has (a) reduced hepatocellular carcinoma (HCC) formation, (b) greater glucose lowering activity, (c) less lipid increasing activity, or (d) less triglyceride, cholesterol, non-HDL or HDL increasing activity compared to FGF19, or a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the WGDPI (SEQ ID NO:170) sequence at amino acids 16-20 of FGF19.

163. The method of claim 137, wherein the N-terminal region comprises any one of the following sequences: MDSSPL (SEQ ID NO:119), MSDSSPL (SEQ ID NO:120), SDSSPL (SEQ ID NO:112), MSSPL (SEQ ID NO:113), or SSPL (SEQ ID NO:114).

164. The method of claim 137, wherein the peptide sequence has (a) reduced hepatocellular carcinoma (HCC) formation, (b) greater glucose lowering activity, (c) less lipid increasing activity, or (d) less triglyceride, cholesterol, non-HDL or HDL increasing activity compared to FGF19, or a FGF19 variant sequence having any of GQV, GDI, WGPI (SEQ ID NO:171), WGDPV (SEQ ID NO:172), WGDI (SEQ ID NO:173), GDPI (SEQ ID NO:174), GPI, WGQPI (SEQ ID NO:175), WGAPI (SEQ ID NO:176), AGDPI (SEQ ID NO:177), WADPI (SEQ ID NO:178), WGDAI (SEQ ID NO:179), WGDPA (SEQ ID NO:180), WDPI (SEQ ID NO:181), WGDI (SEQ ID NO:182), WGDP (SEQ ID NO:183) or FGDPI (SEQ ID NO:184) substituted for the WGDPI (SEQ ID NO:170) sequence at amino acids 16-20 of FGF19.

165. The method of claim 136, wherein the peptide sequence has less lean mass reducing activity compared to FGF21.

166. The method of claim 137, wherein the peptide sequence has less lean mass reducing activity compared to FGF21.

167. The method of claim 141, wherein the constipation is caused by medication or constipation-predominant irritable bowel syndrome.

168. The method of claim 142, wherein the constipation is caused by medication or constipation-predominant irritable bowel syndrome.

169. The method of claim 136, wherein the subject has gastroparesis or diabetes mellitus.

170. The method of claim 137, wherein the subject has gastroparesis or diabetes mellitus.

171. The method of claim 136, wherein the subject has a bile acid associated or related disorder comprising a metabolic syndrome; a lipid or glucose disorder; cholesterol or triglyceride metabolism; type 2 diabetes; cholestasis, intrahepatic cholestasis, primary biliary cirrhosis (PBC), primary familial intrahepatic cholestasis (PFIC), progressive PFIC, primary sclerosing choangitis (PSC), pregnancy intrahepatic cholestasis (PIC), neonatal cholestasis, and drug induced cholestasis, diseases of extrahepatic cholestasis, bile cut compression from tumor, bile duct blockade by gall stones, bile acid malabsorption and other disorders involving the distal small intestine, ileal resection, inflammatory bowel diseases, Crohn's disease, ulcerative colitis, idiopathic disorders impairing absorption of bile acids, diarrhea, bile acid diarrhea (BAD), GI symptoms, GI cancers, liver cancers, biliary cancers, colon cancer, hepatocellular cancer, bile acid synthesis abnormalities, non-alcoholic steatohepatitis (NASH), cirrhosis, portal hypertension, or any combination thereof.

172. The method of claim 137, wherein the subject has a bile acid associated or related disorder comprising a metabolic syndrome; a lipid or glucose disorder; cholesterol or triglyceride metabolism; type 2 diabetes; cholestasis, intrahepatic cholestasis, primary biliary cirrhosis (PBC), primary familial intrahepatic cholestasis (PFIC), progressive PFIC, primary sclerosing choangitis (PSC), pregnancy intrahepatic cholestasis (PIC), neonatal cholestasis, and drug induced cholestasis, diseases of extrahepatic cholestasis, bile cut compression from tumor, bile duct blockade by gall stones, bile acid malabsorption and other disorders involving the distal small intestine, ileal resection, inflammatory bowel diseases, Crohn's disease, ulcerative colitis, idiopathic disorders impairing absorption of bile acids, diarrhea, bile acid diarrhea (BAD), GI symptoms, GI cancers, liver cancers, biliary cancers, colon cancer, hepatocellular cancer, bile acid synthesis abnormalities, non-alcoholic steatohepatitis (NASH), cirrhosis, portal hypertension, or any combination thereof.

173. A method of treating a subject having a gastrointestinal motility-related disorder comprising
(A) genotyping said subject to determine the presence of the KLB minor allele rs17618244; and
(B) administering a therapeutically effective amount of a peptide sequence to the subject, wherein
(a) the peptide sequence comprises
i) an N-terminal region comprising at least seven amino acid residues, the N-terminal region having a first amino acid position and a last amino acid position, wherein the N-terminal region comprises DSSPL (SEQ ID NO:121) or DASPH (SEQ ID NO:122), and
ii) a C-terminal region comprising a portion of SEQ ID NO:99 (FGF19), the C-terminal region having a first amino acid position and a last amino acid position, wherein the C-terminal region comprises amino acid residues 16-29 of SEQ ID NO:99 (FGF19), WGDPIRLRHLYTSG (SEQ ID NO:169), wherein the W residue corresponds to the first amino acid position of the C-terminal region;
(b) the peptide sequence comprises
i) an N-terminal region comprising a portion of SEQ ID NO:100 (FGF21), the N-terminal region having a first amino acid position and a last amino acid position, wherein the N-terminal region comprises amino acid residues GQV, and wherein the V residue corresponds to the last amino acid position of the N-terminal region, and
ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region;
(c) the peptide sequence comprises
i) an N-terminal region comprising a portion of SEQ ID NO:100 (FGF21), the N-terminal region having a first amino acid position and a last amino acid position, wherein the N-terminal region comprises at least 5 contiguous amino acids of SEQ ID NO:100 (FGF21) including the amino acid residues GQV, and wherein the V residue corresponds to the last amino acid position of the N-terminal region, and
ii) a C-terminal region comprising a portion of SEQ ID NO:99 (FGF19), the C-terminal region having a first amino acid position and a last amino acid position, wherein the C-terminal region comprises amino acid residues 21-29 of SEQ ID NO:99 (FGF19), RLRHLYTSG (SEQ ID NO:185), and wherein the R residue corresponds to the first position of the C-terminal region; or
(d) the peptide sequence comprises or consists of
i) a FGF19 sequence variant having one or more amino acid substitutions, insertions or deletions compared to a reference or wild type FGF19;
ii) a FGF21 sequence variant having one or more amino acid substitutions, insertions or deletions compared to a reference or wild type FGF21;
iii) a portion of a FGF19 sequence fused to a portion of a FGF21 sequence; or
iv) a portion of a FGF19 sequence fused to a portion of a FGF21 sequence, wherein the FGF19 and/or FGF21 sequence portion(s) have one or more amino acid substitutions, insertions or deletions compared to a reference or wild type FGF19 and/or FGF21.

174. The method of claim 173, wherein genotyping comprises single nucleotide polymorphism (SNP) assay, restriction fragment length polymorphism identification (RFLPI), random amplified polymorphic detection (RAPD), amplified fragment length polymorphism detection (AFLPD), polymerase chain reaction (PCR), DNA sequencing, DNA microarrays, mass spectrometry (MS), or denaturing high-performance liquid chromatography (DHPLC).

175. The method of claim 173, wherein the peptide sequence is fused to a Fc region.

176. The method of claim 173, wherein the peptide sequence comprises or consists of SEQ ID NO: 69.

177. The method of claim 173, wherein the peptide sequence comprises or consists of SEQ ID NO: 70.

178. The method of claim 173, wherein the subject is heterozygous for KLB minor allele rs17618244.

179. The method of claim 173, wherein the subject is homozygous for KLB minor allele rs17618244.

180. The method of claim 173, wherein the gastrointestinal motility-related disorder is constipation.


SEQUENCE LISTING (212 sequences)

SEQ ID NO: 1 - protein, 194 aa, Homo sapiens
RPLAFSDASPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 2 - protein, 194 aa, Homo sapiens
RPLAFSDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 3 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 4 - protein, 194 aa, Homo sapiens
RPLAFSDAGPHVHYAWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 5 - protein, 191 aa, Homo sapiens
RHPIPDSSPLLQFGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 6 - protein, 187 aa, Homo sapiens
RDSSPLLQFGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 7 - protein, 192 aa, Homo sapiens
RPLAFSDSSPLLQFGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 8 - protein, 191 aa, Homo sapiens
RHPIPDSSPLLQWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 9 - protein, 193 aa, Homo sapiens
RHPIPDSSPLLQFGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 10 - protein, 193 aa, Homo sapiens
RHPIPDSSPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 11 - protein, 192 aa, Homo sapiens
RPLAFSDAGPLLQWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 12 - protein, 194 aa, Homo sapiens
RPLAFSDAGPLLQFGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 13 - protein, 192 aa, Homo sapiens
RPLAFSDAGPLLQFGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 14 - protein, 191 aa, Homo sapiens
RHPIPDSSPHVHYGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 15 - protein, 192 aa, Homo sapiens
RPLAFSDAGPHVHYGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 16 - protein, 192 aa, Homo sapiens
RPLAFSDAGPHVHWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 17 - protein, 192 aa, Homo sapiens
RPLAFSDAGPHVGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 18 - protein, 192 aa, Homo sapiens
RPLAFSDAGPHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 19 - protein, 192 aa, Homo sapiens
RPLAFSDAGPVYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 20 - protein, 192 aa, Homo sapiens
RPLAFSDAGPVHGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 21 - protein, 192 aa, Homo sapiens
RPLAFSDAGPVHYWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 22 - protein, 193 aa, Homo sapiens
RPLAFSDAGPHVHGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 23 - protein, 192 aa, Homo sapiens
RPLAFSDAGPHHGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 24 - protein, 192 aa, Homo sapiens
RPLAFSDAGPHHYWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 25 - protein, 192 aa, Homo sapiens
RPLAFSDAGPHVYWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 26 - protein, 192 aa, Homo sapiens
RPLAFSDSSPLVHWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 27 - protein, 192 aa, Homo sapiens
RPLAFSDSSPHVHWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 28 - protein, 191 aa, Homo sapiens
RPLAFSDAGPHVWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 29 - protein, 193 aa, Homo sapiens
RPLAFSDAGPHVHYWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 30 - protein, 194 aa, Homo sapiens; identical to SEQ ID NO: 4

SEQ ID NO: 31 - protein, 191 aa, Homo sapiens
RHPIPDSSPLLQFGAQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 32 - protein, 191 aa, Homo sapiens
RHPIPDSSPLLQFGDQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 33 - protein, 191 aa, Homo sapiens
RHPIPDSSPLLQFGPQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 34 - protein, 191 aa, Homo sapiens
RHPIPDSSPLLQFGGAVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 35 - protein, 191 aa, Homo sapiens
RHPIPDSSPLLQFGGEVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 36 - protein, 191 aa, Homo sapiens
RHPIPDSSPLLQFGGNVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 37 - protein, 191 aa, Homo sapiens
RHPIPDSSPLLQFGGQARLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 38 - protein, 191 aa, Homo sapiens
RHPIPDSSPLLQFGGQIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 39 - protein, 191 aa, Homo sapiens
RHPIPDSSPLLQFGGQTRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 40 - protein, 193 aa, Homo sapiens
RHPIPDSSPLLQFGWGQPVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 41 - protein, 182 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPEPPGILAPQPPDVGSSDPLSMVGPSQGRSPSYAS

SEQ ID NO: 42 - protein, 178 aa, Homo sapiens
HPIPDSSPLLQFGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPEPPGILAPQPPDVGSSDPLSMVGPSQGRSPSYAS

SEQ ID NO: 43 - protein, 192 aa, Homo sapiens
RPLAFSDAGPHVHYGGDIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 44 - protein, 185 aa, Homo sapiens
RPLAFSDAGPHVHYGWGDPIRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 45 - protein, 193 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPMVPEEPEDLRGHLESDMFSSPLETDSMDPFGLVTGLEAVRSPSFEK

SEQ ID NO: 46 - protein, 232 aa, Homo sapiens
RPLAFSDAGPHVHYGWGDPIRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYASPMVPEEPEDLRGHLESDMFSSPLETDSMDPFGLVTGLEAVRSPSFEK

SEQ ID NO: 47 - protein, 190 aa, Homo sapiens
HPIPDSSPLLQWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 48 - protein, 187 aa, Homo sapiens; identical to SEQ ID NO: 6

SEQ ID NO: 49 - protein, 192 aa, Homo sapiens; identical to SEQ ID NO: 7

SEQ ID NO: 50 - protein, 191 aa, Homo sapiens
RHPIPDSSPLLQFGDQVRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 51 - protein, 191 aa, Homo sapiens; identical to SEQ ID NO: 36

SEQ ID NO: 52 - protein, 187 aa, Homo sapiens
RDSSPLLQWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 53 - protein, 189 aa, Homo sapiens
MDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 54 - protein, 192 aa, Homo sapiens; identical to SEQ ID NO: 11

SEQ ID NO: 55 - protein, 192 aa, Homo sapiens; identical to SEQ ID NO: 18

SEQ ID NO: 56 - protein, 192 aa, Homo sapiens; identical to SEQ ID NO: 19

SEQ ID NO: 57 - protein, 192 aa, Homo sapiens; identical to SEQ ID NO: 20

SEQ ID NO: 58 - protein, 192 aa, Homo sapiens; identical to SEQ ID NO: 21

SEQ ID NO: 59 - protein, 192 aa, Homo sapiens; identical to SEQ ID NO: 23

SEQ ID NO: 60 - protein, 192 aa, Homo sapiens; identical to SEQ ID NO: 24

SEQ ID NO: 61 - protein, 192 aa, Homo sapiens; identical to SEQ ID NO: 17

SEQ ID NO: 62 - protein, 192 aa, Homo sapiens; identical to SEQ ID NO: 25

SEQ ID NO: 63 - protein, 192 aa, Homo sapiens; identical to SEQ ID NO: 16

SEQ ID NO: 64 - protein, 192 aa, Homo sapiens; identical to SEQ ID NO: 26

SEQ ID NO: 65 - protein, 192 aa, Homo sapiens; identical to SEQ ID NO: 27

SEQ ID NO: 66 - protein, 192 aa, Homo sapiens
RPLAFSDAGPHLQWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 67 - protein, 191 aa, Homo sapiens; identical to SEQ ID NO: 28

SEQ ID NO: 68 - protein, 193 aa, Homo sapiens; identical to SEQ ID NO: 29

SEQ ID NO: 69 - protein, 189 aa, Homo sapiens
RDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 70 - protein, 190 aa, Homo sapiens
MRDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 71 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHSLPLHLPGNKSPHRDPAPRGPARFLPLPGLPPALPEPPGILAPQPPDVGSSDPLSMVGPSQGRSPSYAS

SEQ ID NO: 72 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 73 - protein, 212 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVVQDELQGVGGEGCHMHPENCKTLLTDIDRTHTEKPVWDGITGE

SEQ ID NO: 74 - protein, 189 aa, Homo sapiens
RDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 75 - protein, 184 aa, Homo sapiens
RVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 76 - protein, 179 aa, Homo sapiens
RGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 77 - protein, 175 aa, Homo sapiens
RRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 78 - protein, 188 aa, Homo sapiens
RAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 79 - protein, 187 aa, Homo sapiens
RGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 80 - protein, 186 aa, Homo sapiens
RPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 81 - protein, 185 aa, Homo sapiens
RHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 82 - protein, 194 aa, Homo sapiens
RPLAFSAAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 83 - protein, 194 aa, Homo sapiens
RPLAFSDAAPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 84 - protein, 194 aa, Homo sapiens
RPLAFSDAGAHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 85 - protein, 194 aa, Homo sapiens
RPLAFSDAGPHVHYGAGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 86 - protein, 194 aa, Homo sapiens
RPLAFSDAGPHVHYGWGAPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 87 - protein, 167 aa, Homo sapiens
RPLAFSDAGPHVHYGWGDAICARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 88 - protein, 194 aa, Homo sapiens
RPLAFSDAGPHVHYGWGDPIRLRHLYTSGPAGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLAHFLPMLPMVPEEPEDLRGHLESDMFSSPLETDSMDPFGLVTGLEAVRSPSFEK

SEQ ID NO: 89 - protein, 194 aa, Homo sapiens
RPLAFSDAGPHVHYGWGDPIRLRHLYTSGPAGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSAFLPMLPMVPEEPEDLRGHLESDMFSSPLETDSMDPFGLVTGLEAVRSPSFEK

SEQ ID NO: 90 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAAQAQLYKNRGFLPLSHFLPMLPMVPEEPEDLRGHLESDMFSSPLETDSMDPFGLVTGLEAVRSPSFEK

SEQ ID NO: 91 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAAQRQLYKNRGFLPLAHFLPMLPMVPEEPEDLRGHLESDMFSSPLETDSMDPFGLVTGLEAVRSPSFEK

SEQ ID NO: 92 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAAQRQLYKNRGFLPLSAFLPMLPMVPEEPEDLRGHLESDMFSSPLETDSMDPFGLVTGLEAVRSPSFEK

SEQ ID NO: 93 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQAQLYKNRGFLPLAHFLPMLPMVPEEPEDLRGHLESDMFSSPLETDSMDPFGLVTGLEAVRSPSFEK

SEQ ID NO: 94 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLAAFLPMLPMVPEEPEDLRGHLESDMFSSPLETDSMDPFGLVTGLEAVRSPSFEK

SEQ ID NO: 95 - protein, 194 aa, Homo sapiens; identical to SEQ ID NO: 92

SEQ ID NO: 96 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAAQAQLYKNRGFLPLAHFLPMLPMVPEEPEDLRGHLESDMFSSPLETDSMDPFGLVTGLEAVRSPSFEK

SEQ ID NO: 97 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAAQAQLYKNRGFLPLSAFLPMLPMVPEEPEDLRGHLESDMFSSPLETDSMDPFGLVTGLEAVRSPSFEK

SEQ ID NO: 98 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAAQAQLYKNRGFLPLAAFLPMLPMVPEEPEDLRGHLESDMFSSPLETDSMDPFGLVTGLEAVRSPSFEK

SEQ ID NO: 99 - protein, 194 aa, Homo sapiens
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 100 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 101 - protein, 4 aa, Homo sapiens
VHYG

SEQ ID NO: 102 - protein, 9 aa, Homo sapiens
DASPHVHYG

SEQ ID NO: 103 - protein, 9 aa, Homo sapiens
DSSPLVHYG

SEQ ID NO: 104 - protein, 7 aa, Homo sapiens
DSSPLLQ

SEQ ID NO: 105 - protein, 12 aa, Homo sapiens
DSSPLLQFGGQV

SEQ ID NO: 106 - protein, 5 aa, Homo sapiens
RHPIP

SEQ ID NO: 107 - protein, 4 aa, Homo sapiens
HPIP

SEQ ID NO: 108 - protein, 5 aa, Homo sapiens
RPLAF

SEQ ID NO: 109 - protein, 4 aa, Homo sapiens
PLAF

SEQ ID NO: 110 - protein, 6 aa, Homo sapiens
MDSSPL

SEQ ID NO: 111 - protein, 7 aa, Homo sapiens
MSDSSPL

SEQ ID NO: 112 - protein, 6 aa, Homo sapiens
SDSSPL

SEQ ID NO: 113 - protein, 5 aa, Homo sapiens
MSSPL

SEQ ID NO: 114 - protein, 4 aa, Homo sapiens
SSPL

SEQ ID NO: 115 - protein, 4 aa, Homo sapiens
RDSS

SEQ ID NO: 116 - protein, 4 aa, Homo sapiens
MDSS

SEQ ID NO: 117 - protein, 5 aa, Homo sapiens
MRDSS

SEQ ID NO: 118 - protein, 5 aa, Homo sapiens
MSSPL

SEQ ID NO: 119 - protein, 6 aa, Homo sapiens
MDSSPL

SEQ ID NO: 120 - protein, 7 aa, Homo sapiens
MSDSSPL

SEQ ID NO: 121 - protein, 5 aa, Homo sapiens
DSSPL

SEQ ID NO: 122 - protein, 5 aa, Homo sapiens
DASPH

SEQ ID NO: 123 - protein, 4 aa, Homo sapiens
RDSS

SEQ ID NO: 124 - protein, 4 aa, Homo sapiens
MDSS

SEQ ID NO: 125 - protein, 5 aa, Homo sapiens
MRDSS

SEQ ID NO: 126 - protein, 6 aa, Homo sapiens
MDSSPL

SEQ ID NO: 127 - protein, 7 aa, Homo sapiens
MSDSSPL

SEQ ID NO: 128 - protein, 5 aa, Homo sapiens
MSSPL

SEQ ID NO: 129 - protein, 5 aa, Artificial Sequence
  Description of Artificial Sequence Linker sequence
GSGGS

SEQ ID NO: 130 - protein, 4 aa, Artificial Sequence
  Description of Artificial Sequence Linker sequence
GGGS

SEQ ID NO: 131 - protein, 4 aa, Artificial Sequence
  Description of Artificial Sequence Linker sequence
GGSG

SEQ ID NO: 132 - protein, 5 aa, Artificial Sequence
  Description of Artificial Sequence Linker sequence
GGSGG

SEQ ID NO: 133 - protein, 5 aa, Artificial Sequence
  Description of Artificial Sequence Linker sequence
GSGSG

SEQ ID NO: 134 - protein, 5 aa, Artificial Sequence
  Description of Artificial Sequence Linker sequence
GSGGG

SEQ ID NO: 135 - protein, 5 aa, Artificial Sequence
  Description of Artificial Sequence Linker sequence
GSSSG

SEQ ID NO: 136 - DNA, 32 bp, Artificial Sequence
  Description of Artificial Sequence Forward primer
ccgactagtcaccatgcggagcgggtgtgtgg

SEQ ID NO: 137 - DNA, 41 bp, Artificial Sequence
  Description of Artificial Sequence Reverse primer
ataagaatgcggccgcttacttctcaaagctgggactcctc

SEQ ID NO: 138 - protein, 186 aa, Homo sapiens
DSSPLLQFGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 139 - protein, 194 aa, Homo sapiens; identical to SEQ ID NO: 1

SEQ ID NO: 140 - protein, 194 aa, Homo sapiens; identical to SEQ ID NO: 2

SEQ ID NO: 141 - protein, 188 aa, Homo sapiens
DSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 142 - protein, 193 aa, Homo sapiens; identical to SEQ ID NO: 9

SEQ ID NO: 143 - protein, 191 aa, Homo sapiens; identical to SEQ ID NO: 8

SEQ ID NO: 144 - protein, 194 aa, Homo sapiens; identical to SEQ ID NO: 12

SEQ ID NO: 145 - protein, 193 aa, Homo sapiens; identical to SEQ ID NO: 10

SEQ ID NO: 146 - protein, 192 aa, Homo sapiens; identical to SEQ ID NO: 13

SEQ ID NO: 147 - protein, 191 aa, Homo sapiens; identical to SEQ ID NO: 14

SEQ ID NO: 148 - protein, 187 aa, Homo sapiens; identical to SEQ ID NO: 6

SEQ ID NO: 149 - protein, 192 aa, Homo sapiens; identical to SEQ ID NO: 7

SEQ ID NO: 150 - protein, 191 aa, Homo sapiens; identical to SEQ ID NO: 31

SEQ ID NO: 151 - protein, 191 aa, Homo sapiens; identical to SEQ ID NO: 32

SEQ ID NO: 152 - protein, 191 aa, Homo sapiens; identical to SEQ ID NO: 33

SEQ ID NO: 153 - protein, 191 aa, Homo sapiens; identical to SEQ ID NO: 34

SEQ ID NO: 154 - protein, 191 aa, Homo sapiens; identical to SEQ ID NO: 35

SEQ ID NO: 155 - protein, 191 aa, Homo sapiens; identical to SEQ ID NO: 36

SEQ ID NO: 156 - protein, 191 aa, Homo sapiens; identical to SEQ ID NO: 37

SEQ ID NO: 157 - protein, 191 aa, Homo sapiens; identical to SEQ ID NO: 38

SEQ ID NO: 158 - protein, 191 aa, Homo sapiens; identical to SEQ ID NO: 39

SEQ ID NO: 159 - protein, 193 aa, Homo sapiens; identical to SEQ ID NO: 40

SEQ ID NO: 160 - protein, 190 aa, Homo sapiens
HPIPDSSPLLQFGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 161 - protein, 186 aa, Homo sapiens; identical to SEQ ID NO: 138

SEQ ID NO: 162 - protein, 190 aa, Homo sapiens; identical to SEQ ID NO: 47

SEQ ID NO: 163 - protein, 192 aa, Homo sapiens
HPIPDSSPLLQFGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 164 - protein, 192 aa, Homo sapiens
HPIPDSSPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 165 - protein, 190 aa, Homo sapiens
HPIPDSSPHVHYGGQVRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 166 - protein, 188 aa, Homo sapiens
DAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 167 - protein, 183 aa, Homo sapiens
VHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 168 - protein, 174 aa, Homo sapiens
RLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 169 - protein, 14 aa, Artificial Sequence
  Synthetic peptide
WGDPIRLRHLYTSG

SEQ ID NO: 170 - protein, 5 aa, Artificial Sequence
  Synthetic peptide
WGDPI

SEQ ID NO: 171 - protein, 4 aa, Artificial Sequence
  Synthetic peptide
WGPI

SEQ ID NO: 172 - protein, 5 aa, Artificial Sequence
  Synthetic peptide
WGDPV

SEQ ID NO: 173 - protein, 4 aa, Artificial Sequence
  Synthetic peptide
WGDI

SEQ ID NO: 174 - protein, 4 aa, Artificial Sequence
  Synthetic peptide
GDPI

SEQ ID NO: 175 - protein, 5 aa, Artificial Sequence
  Synthetic peptide
WGQPI

SEQ ID NO: 176 - protein, 5 aa, Artificial Sequence
  Synthetic peptide
WGAPI

SEQ ID NO: 177 - protein, 5 aa, Artificial Sequence
  Synthetic peptide
AGDPI

SEQ ID NO: 178 - protein, 5 aa, Artificial Sequence
  Synthetic peptide
WADPI

SEQ ID NO: 179 - protein, 5 aa, Artificial Sequence
  Synthetic peptide
WGDAI

SEQ ID NO: 180 - protein, 5 aa, Artificial Sequence
  Synthetic peptide
WGDPA

SEQ ID NO: 181 - protein, 4 aa, Artificial Sequence
  Synthetic peptide
WDPI

SEQ ID NO: 182 - protein, 4 aa, Artificial Sequence
  Synthetic peptide
WGDI

SEQ ID NO: 183 - protein, 4 aa, Artificial Sequence
  Synthetic peptide
WGDP

SEQ ID NO: 184 - protein, 5 aa, Artificial Sequence
  Synthetic peptide
FGDPI

SEQ ID NO: 185 - protein, 9 aa, Artificial Sequence
  Synthetic peptide
RLRHLYTSG

SEQ ID NO: 186 - protein, 9 aa, Artificial Sequence
  core sequence
RQRYLYTDD

SEQ ID NO: 187 - protein, 13 aa, Artificial Sequence
  Synthetic peptide
AGPHVHYGWGDPI

SEQ ID NO: 188 - protein, 165 aa, Homo sapiens
  FGF19 C-terminal sequence
PHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 189 - protein, 5 aa, Artificial Sequence
  Linker sequence
GGGSG

SEQ ID NO: 190 - protein, 11 aa, Homo sapiens
  Sheet-8/Loop-8/Sheet-9 region of FGF19
EEIRPDGYNVY

SEQ ID NO: 191 - protein, 11 aa, Homo sapiens
  Sheet-8/Loop-8/Sheet-9 region of FGF21
ELLLEDGYNVY

SEQ ID NO: 192 - protein, 187 aa, Artificial Sequence
  M53 sequence
MDSSPLLQWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 193 - protein, 194 aa, Artificial Sequence
  M139 sequence
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEILPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 194 - protein, 194 aa, Artificial Sequence
  M140 sequence
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIREDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 195 - protein, 194 aa, Artificial Sequence
  M141 sequence
RPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEILCDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 196 - protein, 194 aa, Artificial Sequence
  M160 sequence
RPLAFSDAGPHVHYGWGDPIRQRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 197 - protein, 189 aa, Artificial Sequence
  M200 Sequence
RDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 198 - protein, 194 aa, Artificial Sequence
  M201 Sequence
RPLAFSDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 199 - protein, 194 aa, Artificial Sequence
  M202 Sequence
RPLAFSDASPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 200 - protein, 187 aa, Artificial Sequence
  M203 Sequence
RDSSPLLQWGDPIRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 201 - protein, 191 aa, Artificial Sequence; identical to SEQ ID NO: 50
  M204 Sequence

SEQ ID NO: 202 - protein, 187 aa, Artificial Sequence
  M205 Sequence
RDSSPLLQFGGQVRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 203 - protein, 191 aa, Artificial Sequence
  M206 Sequence
RHPIPDSSPLLQFGGQVRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 204 - protein, 190 aa, Artificial Sequence
  M207 Sequence
MRDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEILEDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 205 - protein, 11 aa, Artificial Sequence
  Modified Sheet-8/Loop-8/Sheet-9 region of FGF19
EEILEDGYNVY

SEQ ID NO: 206 - protein, 5 aa, Artificial Sequence
  Linker sequence
GSGGS

SEQ ID NO: 207 - protein, 4 aa, Artificial Sequence
  Linker sequence
GGGS

SEQ ID NO: 208 - protein, 4 aa, Artificial Sequence
  Linker sequence
GGSG

SEQ ID NO: 209 - protein, 5 aa, Artificial Sequence
  Linker sequence
GGSGG

SEQ ID NO: 210 - protein, 5 aa, Artificial Sequence
  Linker sequence
GSGSG

SEQ ID NO: 211 - protein, 5 aa, Artificial Sequence
  Linker sequence
GSGGG

SEQ ID NO: 212 - protein, 5 aa, Artificial Sequence
  Linker sequence
GSSSG